(19) United States
(12) Patent Application Publication
(10) Pub. No.: US 2026/0273057 A1
(43) Pub. Date: Sep. 17, 2026

(54) METHODS AND COMPOSITIONS FOR TREATING AUTOIMMUNE DISEASES

(71) Applicant: CABALETTA BIO, INC., Philadelphia, PA (US)

(72) Inventors: Gwendolyn Knowlton Binder, Philadelphia, PA (US); David Chang, Bryn Mawr, PA (US); Samik Basu, Philadelphia, PA (US); Steven Alan Nichtberger, Villanova, PA (US); Georg Schett, Erlangen (DE); Andreas Mackensen, Erlangen (DE)

(21) Appl. No.: 19/470,700

(22) PCT Filed: Mar. 29, 2024
(86) PCT No.: PCT/US2024/022338

§ 371 Date: Sep. 29, 2025

Related U.S. Application Data
(60) Provisional application No. 63/493,136, filed on Mar. 30, 2023.

(51) Int. Cl. A61K 40/31; A61K 31/675; A61K 31/7076; A61K 40/11; A61K 40/42; A61P 37/06

(52) U.S. Cl. CPC A61K 40/31; A61K 31/675; A61K 31/7076; A61K 40/11; A61K 40/4211; A61P 37/06

(57) ABSTRACT

The invention relates generally to methods of treating autoimmune disease, comprising administering a preconditioning regimen comprising a dose of cyclophosphamide and a dose of fludarabine to a subject, and subsequently administering a T cell therapy.

### CROSS REFERENCE TO RELATED APPLICATIONS

This application claims the benefit of and priority to U.S. Provisional Application No. 63/493,136, filed Mar. 30, 2023, the entire disclosure of which is hereby incorporated by reference in its entirety for all purposes.

### SEQUENCE LISTING

This application contains a Sequence Listing which has been submitted electronically in XML format and is hereby incorporated by reference in its entirety. The XML copy, created on Mar. 28, 2024, is named CBB-202WO_SL.xml and is 57.9 kilobytes in size.

### FIELD OF THE INVENTION

The invention relates generally to methods of using immune cell therapy to treat autoimmune diseases wherein, prior to administration of the immune cell therapy to a subject in need thereof, the subject is administered a preconditioning regimen comprising a dose of cyclophosphamide and a dose of fludarabine.

### BACKGROUND

According to the National Institute of Health, autoimmune diseases affect more than 23.5 million people in the United States. Autoimmune diseases are a group of diseases which result from body's immune system attacking healthy cells or tissues. In the case of autoimmune diseases where B cells have a role in initiating or maintaining the disease, certain populations of a patient's B cells differentiate into antibody secreting cells that produce antibodies directed against normal tissues and cells. While these autoantibodies are the effectors of such diseases, the underlying cause is defective B cells that mistakenly differentiate into cells that secrete these pathogenic antibodies.

Current treatment options for autoimmune mediated diseases involve generalized immune suppression, achieved through corticosteroids, immunosuppressive medications, and biologics. Most commonly, corticosteroids are used on both a chronic and acute basis to control disease and act via a variety of mechanisms to control or downregulate multiple inflammatory pathways. In many cases, systemic immunosuppressive medications such as mycophenolate, azathioprine and methotrexate, are added in an effort to minimize symptoms and manage the expected recurrences in patients. Most therapies for treating autoimmune diseases are not curative and require chronic administration, often for the lifetime of patient. Furthermore, there is the risk of serious and/or life-threatening infection for patients receiving chronic immunosuppressive therapy. As such, there is a significant unmet medical need for these patients.

Over the course of the last decade, chimeric antigen receptor (CAR) T cell therapy has become an established treatment for a variety of cancers. The method has seen particular success in haematological malignancies such as acute lymphoblastic leukemia (ALL) and diffuse large B-cell lymphoma (DLBCL). In general, CAR T cell therapy for the treatment of cancer involves isolating T cells from human blood, genetically engineering the cells to express a CAR against a desired target antigen, administering a lymphodepleting chemotherapeutic preconditioning regimen to the patient, and subsequently administering the engineered CAR T cells to the patient, thereby allowing the CAR T cells to target and destroy cancerous cells expressing this antigen. Lymphodepleting preconditioning has been considered to be an integral part of CAR T cell therapy for cancer patients, and it has been demonstrated that such a preconditioning regimen enhances the survival, persistence, and efficacy of the administered CAR T cells, for example, by increasing levels of homeostatic cytokines such as IL-7 and IL-15, and by eliminating endogenous immune cells competing for said cytokines, thereby creating a more favorable environment for the infused cells to proliferate (Nissani et al. (2021) J. IMMUNOTHER. CANCER 9: e001743).

More recently, CAR T cell therapy has also been investigated as treatment for refractory systemic lupus erythematosus (SLE), a B-cell-mediated autoimmune disease. Researchers reported that administration of a CAR T cell therapy induced clinical remission in 5 out of 5 patients with moderate to severe, refractory SLE (Mackensen et al. (2022) NAT. MED. 28: 2124-2132). In this study, patients received a lymphodepleting preconditioning regimen prior to the CAR T cell therapy, comprising a total dose of 75 mg/m² fludarabine (25 mg/m² on Days −5, −4, and −3) and a total dose of 1,000 mg/m² cyclophosphamide (1,000 mg/m² on Day −3; Mackensen, supra). Similar findings were reported in a case study of a patient with antisynthetase syndrome (myositis) (Muller et al. (2022) 401:815-818 LANCET). The requirement for the preconditioning regimen in achieving a successful therapeutic outcome was not investigated in either study.

Although a lymphodepleting preconditioning is understood to be an important step in engineered T cell therapy for the treatment of cancer, the degree and intensity of lymphodepleting preconditioning required to enable successful treatment of autoimmune diseases with T cell therapy is not understood. Accordingly, in spite of the advancements made to date, there remains a need for new and useful treatments for treating autoimmune diseases.

### SUMMARY OF THE INVENTION

Although preconditioning regimens have been used in engineered immune-cell therapies for treating cancer, given the potential mortality of subjects with cancer, aggressive preconditioning regimens have been considered to pose an acceptable risk to the patients. However, autoimmune diseases generally do not pose the same imminent life-threatening risk as cancer. As a result, preconditioning treatments suitable for treating cancer may not be as appropriate for treating patients with autoimmune diseases, and thus the associated risk of such preconditioning treatments may not be acceptable to the patients and their physicians. It has now been discovered that using lower doses of preconditioning agents is effective when using immune-cell therapies for treating autoimmune disease, without posing the risks associated with using higher doses of the agents used with cancer patients. Preconditioning regimens described herein, which utilize a reduced dose of cyclophosphamide and/or a reduced dose fludarabine as compared to a standard preconditioning regimen, were surprisingly found to reduce levels of endogenous immune cells to a sufficient degree to enable successful treatment of autoimmune diseases with immune cell therapy, while minimizing the toxicity and risks normally associated with cyclophosphamide and fludarabine treatment.

Accordingly, in one aspect, the present disclosure provides a method of treating an autoimmune disease in a subject in need thereof, the method comprising: (a) administering to the subject a preconditioning regimen comprising a dose of 100-1,500 mg/m²/day cyclophosphamide and a dose of 2-20 mg/m²/day fludarabine; and (b) after step (a), administering to the subject a therapeutically effective amount of engineered CAR immune cells.

In certain embodiments, the dose of cyclophosphamide is less than 1,000 mg/m²/day, less than 800 mg/m²/day, less than 600 mg/m²/day, or less than 500 mg/m²/day. In certain embodiments, the dose of cyclophosphamide is 200-800 mg/m²/day, for example, 400-600 mg/m²/day.

In certain embodiments, the dose of cyclophosphamide is 1,000 mg/m²/day, 500 mg/m²/day, or 250 mg/m²/day.

In certain embodiments, the preconditioning regimen comprises administering a total dose of cyclophosphamide of less than 2,000 mg/m², less than 1,000 mg/m², less than 800 mg/m², less than 600 mg/m², or less than 500 mg/m² to the subject. In certain embodiments, the preconditioning regimen comprises administering a total dose of 100-1,500 mg/m² cyclophosphamide to the subject. In certain embodiments, the preconditioning regimen comprises administering a total dose of 250-1,000 mg/m² cyclophosphamide to the subject.

In certain embodiments, the preconditioning regimen comprises administering a total dose of 1,000 mg/m², 500 mg/m², or 250 mg/m² cyclophosphamide to the subject.

In certain embodiments, the subject is administered less than 2,000 mg/m², less than 1,000 mg/m², less than 800 mg/m², less than 600 mg/m², or less than 500 mg/m² cyclophosphamide in total during the seven days prior to step (b).

In certain embodiments of any of the foregoing methods, the dose of fludarabine is less than 15 mg/m²/day, less than 12.5 mg/m²/day, or less than 10 mg/m²/day. In certain embodiments, the dose of fludarabine is 5-15 mg/m²/day, for example, 10-15 mg/m²/day.

In certain embodiments, the dose of fludarabine is 15 mg/m²/day, 12.5 mg/m²/day, or 6.25 mg/m²/day.

In certain embodiments, the preconditioning regimen comprises administering a total dose of fludarabine of less than 75 mg/m², less than 50 mg/m², or less than 25 mg/m² to the subject. In certain embodiments, the preconditioning regimen comprises administering a total dose of 5-60 mg/m² or 15-50 mg/m² fludarabine to the subject.

In certain embodiments, the preconditioning regimen comprises administering a total dose of 45 mg/m², a total dose of 37.5 mg/m², or a total dose of 18.75 mg/m² fludarabine to the subject.

In certain embodiments, the subject is administered less than less than 75 mg/m², less than 50 mg/m², or less than 25 mg/m² fludarabine in total during the seven days prior to step (b).

In certain embodiments of any of the foregoing methods, the dose of cyclophosphamide and/or the dose of fludarabine are administered between 1 and 7 days prior to step (b). In certain embodiments, the dose of cyclophosphamide and the dose of fludarabine are administered on the same day. In certain embodiments, the dose of cyclophosphamide is administered to the subject 3 days and/or 4 days prior to step (b). In certain embodiments, the dose of fludarabine is administered daily for three days prior to step (b). In certain embodiments, the dose of fludarabine is administered 5 days, 4 days, and/or 3 days prior to step (b).

In certain embodiments, the preconditioning regimen comprises or consists essentially of (i) administering the dose of fludarabine to the subject 5 days, 4 days, and 3 days prior to step (b), and (ii) administering the dose of cyclophosphamide to the subject 3 days prior to step (b). In certain embodiments, the dose of fludarabine is 12.5 mg/m²/day and the dose of cyclophosphamide is 500 mg/m²/day. In certain embodiments, the dose of fludarabine is 15 mg/m²/day and the dose of cyclophosphamide is 500 mg/m²/day. In certain embodiments, the dose of fludarabine is 12.5 mg/m²/day and the dose of cyclophosphamide is 1,000 mg/m²/day. In certain embodiments, the dose of fludarabine is 6.25 mg/m²/day and the dose of cyclophosphamide is 250 mg/m²/day. In certain embodiments, the dose of fludarabine is 6.25 mg/m²/day and the dose of cyclophosphamide is 500 mg/m²/day. In certain embodiments, the dose of fludarabine is 6.25 mg/m²/day and the dose of cyclophosphamide is 1,000 mg/m²/day.

In certain embodiments, the preconditioning regimen comprises or consists essentially of (i) administering the dose of fludarabine to the subject 5 days, 4 days, and 3 days prior to step (b), and (ii) administering the dose of cyclophosphamide to the subject 4 days and 3 days prior to step (b). In certain embodiments, the dose of fludarabine is 6.25 mg/m²/day and the dose of cyclophosphamide is 500 mg/m²/day.

In certain embodiments, the preconditioning regimen is a regimen as described in TABLE 1.

In certain embodiments of any of the foregoing methods, the therapeutically effective amount of engineered CAR immune cells is between 1×10⁵ cells/kg and 1×10⁸ cells/kg, for example, between 1×10⁶ cells/kg and 1×10⁷ cells/kg.

In certain embodiments of any of the foregoing methods, the engineered CAR immune cells express a CAR which specifically binds a B-cell surface antigen, for example, CD19.

In certain embodiments of any of the foregoing methods, the engineered CAR immune cells comprise a nucleic acid encoding a CAR, wherein the CAR comprises: (i) an extracellular domain comprising an antigen binding site, for example, an scFv; (ii) a transmembrane domain; (iii) a costimulatory domain; and (iv) an intracellular signaling domain.

In certain embodiments, the antigen binding site comprises a heavy chain variable domain (V_{H}) comprising complementarity determining regions CDRH1, CDRH2, and CDRH3 and a light chain variable domain (V_{L}) comprising complementarity determining regions CDRL1, CDRL2, and CDRL3, wherein: (i) the CDRH1, CDRH2, and CDRH3 comprise the amino acid sequences of SEQ ID NOs: 1, 2, and 3, respectively; and/or the CDRL1, CDRL2, and CDRL3 comprise the amino acid sequences of SEQ ID NOs: 5, 6, and 7, respectively; or (ii) the CDRH1, CDRH2, and CDRH3 comprise the amino acid sequences of SEQ ID NOs: 10, 11, and 12, respectively; and/or the CDRL1, CDRL2, and CDRL3 comprise the amino acid sequences of SEQ ID NOs: 14, 15, and 16, respectively. In certain embodiments, (i) the V_{H} and the V_{L} comprise amino acid sequences at least 95% (e.g., at least 95%, 96%, 97%, 98%, 99%, or 99.5%) identical to the amino acid sequences of SEQ ID NOs: 4 and 8, respectively; or (ii) the VH and the VL comprise amino acid sequences at least 95% (e.g., at least 95%, 96%, 97%, 98%, 99%, or 99.5%) identical to the amino acid sequences of SEQ ID NOs: 13 and 17, respectively. In certain embodiments, (i) the VH and the VL comprise the amino acid sequences SEQ ID NOs: 4 and 8, respectively; or (ii) the VH and the VL comprise the amino acid sequences of SEQ ID NOs: 13 and 17, respectively.

In certain embodiments, the scFv comprises an amino acid sequence at least 95% (e.g., at least 95%, 96%, 97%, 98%, 99%, or 99.5%) identical to the amino acid sequence of SEQ ID NO: 9 or SEQ ID NO: 18. In certain embodiments, the scFv comprises the amino acid sequence of SEQ ID NO: 9 or SEQ ID NO: 18.

In certain embodiments, the antigen binding site is humanized or fully human.

In certain embodiments, the transmembrane domain comprises a CD8 alpha chain transmembrane domain, e.g., a CD8 alpha chain transmembrane domain comprising the amino acid sequence of SEQ ID NO: 19. In certain embodiments, the costimulatory domain comprises a 4-1BB intracellular domain, e.g., a 4-1BB intracellular domain comprising the amino acid sequence of SEQ ID NO: 20. In certain embodiments, the intracellular signaling domain comprises a CD3ζ signaling domain, e.g., a CD3ζ signaling domain comprising the amino acid sequence of SEQ ID NO: 21. In certain embodiments, the CAR further comprises a hinge or linker interposed between the antigen binding site and transmembrane domain. In certain embodiments, the hinge domain is a CD8 alpha chain hinge, e.g., a CD8 alpha chain hinge comprising the amino acid sequence of SEQ ID NO: 22.

In certain embodiments, the CAR comprises the amino acid sequence of SEQ ID NO: 23.

In certain embodiments of any of the foregoing methods, the autoimmune disease is a B-cell-mediated autoimmune disease, for example, an autoimmune disease selected from the group consisting of systemic lupus erythematosus (SLE), pemphigus vulgaris (PV), myasthenia gravis (MG), myositis, and membranous nephropathy. In certain embodiments, the autoimmune disease is selected from the group consisting of lupus nephritis, SLE with anti-dsDNA antibodies, mucosal PV, mucocutaneous PV, MuSK-associated MG, AChR MG, anti-synthetase syndrome, dermatomyositis, and immune mediated necrotizing myopathy. In certain embodiments of any of the foregoing methods, the autoimmune disease is SLE.

In certain embodiments of any of the foregoing methods, the CAR immune cells are CAR T cells or CAR NK cells. In certain embodiments, the CAR immune cells are CAR T cells.

These and other aspects and features of the present disclosure are described in the following detailed description and claims.

### DESCRIPTION OF THE DRAWINGS

The invention can be more completely understood with reference to the following drawings.

FIG. 1 depicts schematic diagrams of exemplary CD19-CAR constructs.

FIGS. 2A-2C depict schematic diagrams summarizing the Phase I/II clinical study of CD19-CAR T cell therapy for the treatment of SLE described in Example 3. FIG. 2A summarizes the protocol for Part A of the study, which determines the tolerability and biological activity of the CD19-CAR T treatment (CAB-001). FIGS. 2B-2C summarize the protocol for Part B of the study, in which subjects with either lupus nephritis (FIG. 2B) or non-renal SLE (FIG. 2C) are treated with CAB-001 following treatment with a typical, oncology-type preconditioning regimen (Group B1) or a reduced preconditioning regimen (Group B2, starting at 52 weeks). Subjects in Group B2 are initially treated with belimumab with standard of care for 52 weeks as a comparator group.

FIG. 3 depicts schematic diagrams summarizing the Phase I/II clinical study of CD19-CAR T therapy for the treatment of myasthenia gravis described in Example 6, including AChR antibody-positive and AChR antibody-negative cohorts.

FIG. 4 depicts schematic diagrams summarizing the Phase I/II clinical study of CD19-CAR T therapy for the treatment of systemic sclerosis described in Example 7, including a severe skin cohort for subjects with skin involvement who do not meet pulmonary, cardiac, or renal involvement criteria, and an organ cohort for subjects who do meet pulmonary, cardiac, or renal involvement criteria, regardless of skin involvement.

FIG. 5 depicts schematic diagrams summarizing the Phase I/II clinical study of CD19-CAR T therapy for the treatment of myositis (idiopathic inflammatory myopathies, IIMs) described in Example 8, including a dermatomyositis cohort (“DM”), an anti-synthetase syndrome cohort (“ASyS”), and an immune-mediated necrotizing myopathy cohort (“IMNM”).

FIG. 6 depicts schematic diagrams summarizing the Phase I/II clinical study of CD19-CAR T therapy for the treatment of SLE described in Example 9, including a lupus nephritis cohort (“LN”) and a non-renal SLE cohort.

### DETAILED DESCRIPTION

Although preconditioning regimens have been used in engineered and non-engineered immune-cell therapies for treating cancer, given the potential mortality of subjects with cancer, aggressive preconditioning regimens have been considered to pose an acceptable risk to the patients. However, autoimmune diseases generally do not pose the same imminent life-threatening risk as cancer. As a result, preconditioning treatments suitable for treating cancer may not be as appropriate for treating patients with autoimmune diseases, and thus the associated risk of such preconditioning treatments may not be acceptable to the patients and their physicians. It has now been discovered that using lower doses of preconditioning agents is effective when using immune-cell therapies for treating autoimmune disease, without posing the risks associated with using higher doses of the agents used with cancer patients. Preconditioning regimens described herein, which utilize a reduced dose of cyclophosphamide and/or a reduced dose fludarabine as compared to a standard preconditioning regimen, were surprisingly found to reduce levels of endogenous immune cells to a sufficient degree to enable successful treatment of autoimmune diseases with immune cell therapy, while minimizing the toxicity and risks normally associated with cyclophosphamide and fludarabine treatment.

### I. Definitions

To facilitate an understanding of the present invention, a number of terms and phrases are defined below.

The terms “a” and “an” as used herein mean “one or more” and include the plural unless the context is inappropriate.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As used herein, the term “antigen-binding site” refers to an antigen-binding fragment of an immunoglobulin or a derivative or variant thereof that participates in antigen binding. For example, in human antibodies, an antigen-binding site is formed by the N-terminal variable domains of the heavy chain and light chain, which are also called “heavy chain variable domain (VH)” and “light chain variable domain (VL),” respectively. In each of the variable domains, three highly divergent stretches called “hypervariable regions” are interposed between more conserved flanking stretches known as “framework regions” (FRs). The three hypervariable regions of a VH and the three hypervariable regions of a VL are disposed relative to each other in three-dimensional space to form an antigen-binding surface complementary to the three-dimensional surface of a bound antigen. The hypervariable regions are also referred to as “complementarity-determining regions” or “CDRs.” The boundaries of the FRs and CDRs can be defined using any appropriate convention known in the art, including, for example, by the IMGT convention (see, Lefranc, (1999) The Immunologist, 7, 132-136), by Kabat convention (see, Kabat, E. A., et al. (1991) SEQUENCES OF PROTEINS OF IMMUNOLOGICAL INTEREST, FIFTH EDITION, U.S. Department of Health and Human Services, NIH Publication No. 91-3242), or by the Chothia convention (see, Chothia, C. et al. (1987) J. MOL. BIOL. 196:901-917). The three CDRs, referred to as CDR₁, CDR₂, and CDR₃, contribute to the antibody binding specificity.

Examples of antigen-binding fragments of an immunoglobulin include, for example, Fab, Fab′, and F(ab′)₂ fragments. Examples of variants of antigen-binding fragments of immunoglobulins include, for example, single chain antibodies or scFvs. Certain animals have different forms of antibodies. For example, camelids have antibodies comprising V_{H}H fragments and cartilaginous fishes have antibodies called “new antigen receptor immunoglobulins” or “IgNARs” comprising V_{NAR} fragments, where such fragments, which are single, monomeric antibody variable domains that are able to bind selectively to a specific antigen independently of another variable domain, are called “single domain antibody,” “sdAb,” or “nanobody.” An antigen-binding site can comprise either a pair of VH and VL or an sdAb. An antigen-binding site disclosed herein can be recombinant, chimeric, deimmunized, humanized, and/or affinity matured (see, e.g., U.S. Pat. No. 4,816,567; Morrison et al. (1984) PROC. NATL. ACAD. SCI. U.S.A., 81: 6851-55; Morrison et al. (1985) PROC. NATL. ACAD. SCI. U.S.A., 81:6851; Takeda et al. (1985) NATURE, 314: 452).

The term “cross-compete,” as used herein in the context of a subject antibody and a reference antibody, indicates that the subject antibody competes for binding to an antigen (e.g., CD19) with the reference antibody and vice versa. A subject antibody cross-competes with a reference antibody if competition is observed whether the reference antibody is used as the first antibody and the subject antibody is used as the second antibody, or the subject antibody is used as the first antibody and the reference antibody is used as the second antibody in this assay. A skilled artisan can select the concentrations of the antibodies used in the competition assays based on the affinities of the antibodies for the antigen and the valency of the antibodies. In an exemplary assay, a first anti-CD19 antibody is immobilized on a solid surface, CD19 is bound to the first antibody, and binding of a second anti-CD19 antibody is assessed. If the second antibody does not generate a significant binding signal, the second antibody competes with the first antibody for binding CD19. Exemplary assays are described in Cox et al., “Immunoassay Methods,” in ASSAY GUIDANCE MANUAL [INTERNET], Updated Dec. 24, 2014 (www.ncbi.nlm.nih.gov/books/NBK92434/; accessed Sep. 29, 2015); Silman et al. (2001) CYTOMETRY, 44: 30-37; and Finco et al. (2011) J. PHARM. BIOMED. ANAL., 54: 351-358. A CD19 binding agent comprising an antigen-binding site, such as a fragment of, or scFv derived from, an anti-CD19 antibody, can also be assessed in this assay.

Doses described herein can be presented as a “weight based dose” or as a “body surface area (BSA) based dose.” A weight based dose is a dose that is administered to a patient that is calculated based on the weight of the patient, e.g., mg/kg. A BSA based dose is a dose that is administered to a patient that is calculated based on the surface area of the patient, e.g., mg/m². The two forms of dose measurement can be converted for human dosing by multiplying the weight based dose by 37 or dividing the BSA based dose by 37. For example, a dose of 60 mg/kg to be administered to a human subject is equivalent to a 2,220 mg/m² dose of the same drug to be administered to the same subject.

As used herein, the term “effective amount” refers to the amount of a compound or agent (e.g., a compound or agent of the present disclosure) sufficient to effect beneficial or desired results. For example, “effective amount” can refer to the amount of an active agent (e.g., cyclophosphamide, fludarabine, or a CAR immune cell) sufficient to effect beneficial or desired results. For example, an effective amount of an agent may be an amount sufficient to achieve one or more of the following: (1) treat an autoimmune disease, e.g., SLE, pemphigus vulgaris, or myositis; (2) reduce or eliminate circulating B cells in a subject; (3) decrease autoantibody levels in a subject; (4) reduce or eliminate endogenous lymphocytes in a subject; (5) reduce proteinuria in a subject; or (6) increase the amount or activity of one or more complement factors in a subject. An effective amount can be administered in one or more administrations, applications or dosages and is not intended to be limited to a particular formulation or administration route.

As used herein, percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Similarly, percent “identity” between a nucleic acid sequence and a reference sequence is defined as the percentage of nucleotides in the nucleic acid sequence that are identical to the nucleotides in the reference sequence, after aligning the sequences and introducing gaps, if necessary, to achieve the maximum percent sequence identity. Alignment for purposes of determining percent sequence identity (e.g., nucleic acid sequence identity or amino acid sequence identity) can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the term “isolated” when used in conjunction with a particular article (e.g., polypeptide, nucleic acid, or cell) is understood to mean: (1) that the article has been separated or purified from other components (e.g., other proteins, peptides, nucleic acids, cells, or cellular materials) and/or chemicals (e.g., reagents used in manufacture); (2) that the article may be separated or purified from the environment in which it may exist in nature, for example, a tissue or fluid sample; or (3) that the article does not occur in nature. For example, a molecule that is removed from a cell that produces it, is “isolated”. A chemically synthesized molecule is “isolated”. As used herein, the term “isolated” can also refer to a molecule that is substantially free of other molecules of the same species. For example, a protein may be “isolated” from other proteins having different amino acid sequences. The purity or homogeneity of a desired article can be assayed using techniques well known in the art, including gel electrophoresis, high performance liquid chromatography, or mass spectrometry. Any of the polynucleotides, polypeptides, vectors, compounds, or cells described herein may be isolated.

As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers, and adjuvants, see e.g., Adeboye Adejare, Remington: The Science and Practice of Pharmacy (23d ed. 2020).

The term “purified”, as used herein, means that an entity or substance is separated from one or more other entities or substances with which it was previously found before being purified. An entity or substance may be partially purified, substantially purified, or pure. A substance or entity such as a nucleic acid or polypeptide is considered pure when it is removed from substantially all other compounds or entities other than a solvent and any ions contained in the solvent, i.e., it constitutes at least about 90%, more preferably at least about 91%, 92%, 93%, 94%, 95%, 96%, 97%, 98%, 99%, or greater than 99% of the dry weight of the composition. A partially or substantially purified compound or entity such as a nucleic acid or polypeptide may be removed from at least 50%, at least 60%, at least 70%, or at least 80% by weight of the material with which it is naturally found, e.g., cellular material such as cellular proteins and/or nucleic acids. In certain embodiments, the purified nucleic acid or polypeptide constitutes at least 10%, 20%, 30%, 40%, 50%, 60%, 70%, 80%, 90%, 95%, 99% or even more, by dry weight, of the total nucleic acid or polypeptide, respectively, in a composition. Methods for assessing purity are known in the art and include chromatographic methods, immunological methods, electrophoretic methods, etc. Any of the polynucleotides, polypeptides, or cells described herein may be purified.

As used herein, the terms “subject” and “patient” refer to an organism to be treated by any of the methods and compositions described herein. Such organisms preferably include, but are not limited to, mammals (e.g., murines, simians, equines, bovines, porcines, canines, felines, and the like), and more preferably include humans.

As used herein, “treat”, “treating”, and “treatment” refer to the treatment of a disease, disorder, or symptom or manifestation of such in a subject, e.g., in a human. This includes: (a) preventing a disease or disorder, (b) inhibiting the disease, disorder, etc., i.e., slowing or arresting its progress or development; and (c) relieving the disease, disorder, etc., i.e., causing regression of the disease state. As used herein, “prevent”, “preventing” and “prevention” refer to causing a disease, disorder, or symptom or manifestation of such not to occur for at least a period of time in at least some subjects.

### II. Methods

Disclosed herein, in one aspect, is a method of treating an autoimmune disease in a subject in need thereof by administering to the subject a preconditioning regimen for immune cell therapy, wherein the preconditioning regimen comprises a dose of cyclophosphamide and a dose of fludarabine, and subsequently administering to the subject an effective amount of engineered immune cells (e.g., CAR immune cells, e.g., CAR T cells). In a related aspect, the disclosure provides a method of conditioning a subject for immune cell therapy (e.g., CAR T cell therapy), the method comprising administering to the subject a preconditioning regimen comprising a dose of cyclophosphamide and a dose of fludarabine.

### A. Preconditioning Regimens

Cyclophosphamide is a prodrug which has been used in the treatment of various diseases, disorders, and conditioning regimens for treating cancer. Following administration to a subject, cyclophosphamide is understood to be activated by the hepatic cytochrome P-450 enzyme system, thereby forming the intermediate metabolite 4-hydroxycyclophosphamide and its tautomer, aldophosphamide. These two intermediate metabolites may diffuse into cells, where aldophosphamide is subsequently decomposed. In certain instances, aldophosphamide is converted via β-elimination into two molecules, the cytotoxic metabolite phosphoramide mustard and the byproduct acrolein. Alternatively, aldophosphamide can be converted into the inactive, non-toxic metabolite carboxyphosphamide by the activity of aldehyde dehydrogenase (ALDH) enzymes. Accordingly, formation of nontoxic carboxyphosphamide is more likely to occur in cells with high levels of ALDH, such as hematopoietic stem cells and liver cells, whereas formation of the toxic metabolites phosphoramide mustard and acrolein is more likely to occur in cells with low levels of ALDH, such as mature B cells and T cells. Phosphoramide mustard is a bifunctional DNA alkylating molecule which is understood to react with, for example, the N₇ atom within the imidazole ring of DNA guanine nucleobases, thereby forming covalent linkages between, e.g., two guanine residues on the same strand of DNA (an “intrastrand crosslinkage”) or between two guanine residues on different strands of DNA (an “interstrand crosslinkage”). Formation of these DNA crosslinkages interferes with DNA replication, thereby leading to cell death. The acrolein byproduct of phosphoramide mustard formation is also toxic, and can cause hemorrhagic cystitis (Emadi et al. (2009) NAT. REV. CLIN. ONCOL. 6: 638-647).

Cyclophosphamide has been used in the treatment of a variety of indications, including cancers such as lymphoma, multiple myeloma, leukemia, mycosis fungoides, neuroblastoma, ovarian cancer, eye cancer, and breast cancer. Cyclophosphamide has also been used in the treatment of certain autoimmune diseases, as well as in conditioning treatments for allogeneic bone marrow transplantation and for adoptive cell therapy for the treatment of certain cancers.

Fludarabine is a purine analog which differs from physiologic nucleosides in that the sugar moiety is arabinose instead of ribose or deoxyribose. Fludarabine is generally used in its 5-O-phosphorylated form, i.e., fludarabine phosphate. The term “fludarabine” as used herein is understood to encompass both the non-phosphorylated form and the 5-O-phosphorylated form. Fludarabine acts as a purine antagonist antimetabolite. Once administered to a subject, fludarabine is understood to be dephosphorylated to 2-fluoro-ara-A and then phosphorylated intracellularly by deoxycytidine kinase to the active triphosphate, 2-fluoro-ara-ATP. This metabolite then interferes with DNA replication, likely by inhibiting, e.g., DNA polymerase and ribonucleotide reductase, thus inhibiting DNA synthesis (Gandhi and Plunkett (2002) CLIN. PHARMACOKINET. 41: 93-103). As a result, fludarabine administration leads to increased cell death in dividing cells. Fludarabine is used in the treatment of various diseases and disorders, including hematological malignancies such as lymphomas and leukemias. Fludarabine has also been used in conditioning treatments administered to patients prior to allogeneic stem cell transplantation or adoptive cell therapy.

In certain embodiments, the disclosure provides a method of treating autoimmune disorders in a subject in need thereof, the method comprising: (a) administering to the subject a preconditioning regimen comprising a dose of 100-2,000 mg/m²/day cyclophosphamide and a dose of 2-20 mg/m²/day fludarabine; and (b) after step (a), administering to the subject a therapeutically effective amount of engineered immune cells (e.g., CAR immune cells, e.g., CAR T cells).

In certain embodiments, a preconditioning regimen of the disclosure comprises administering to the subject a dose of fludarabine and a dose of cyclophosphamide, wherein the dose of cyclophosphamide is 100-2,000 mg/m²/day. For example, in certain embodiments, the preconditioning regimen comprises administering a dose of 100-2,000 mg/m²/day, 100-1,750 mg/m²/day, 100-1,500 mg/m²/day, 100-1,400 mg/m²/day, 100-1,300 mg/m²/day, 100-1,200 mg/m²/day, 100-1,100 mg/m²/day, 100-1,000 mg/m²/day, 100-950 mg/m²/day, 100-900 mg/m²/day, 100-850 mg/m²/day, 100-800 mg/m²/day, 100-750 mg/m²/day, 100-700 mg/m²/day, 100-650 mg/m²/day, 100-600 mg/m²/day, 100-550 mg/m²/day, 100-500 mg/m²/day, 100-450 mg/m²/day, 100-400 mg/m²/day, 100-375 mg/m²/day, 100-350 mg/m²/day, 100-300 mg/m²/day, 100-250 mg/m²/day, 100-200 mg/m²/day, 100-150 mg/m²/day, 150-2,000 mg/m²/day, 150-1,750 mg/m²/day, 150-1,500 mg/m²/day, 150-1,400 mg/m²/day, 150-1,300 mg/m²/day, 150-1,200 mg/m²/day, 150-1,100 mg/m²/day, 150-1,000 mg/m²/day, 150-950 mg/m²/day, 150-900 mg/m²/day, 150-850 mg/m²/day, 150-800 mg/m²/day, 150-750 mg/m²/day, 150-700 mg/m²/day, 150-650 mg/m²/day, 150-600 mg/m²/day, 150-550 mg/m²/day, 150-500 mg/m²/day, 150-450 mg/m²/day, 150-400 mg/m²/day, 150-375 mg/m²/day, 150-350 mg/m²/day, 150-300 mg/m²/day, 150-250 mg/m²/day, 150-200 mg/m²/day, 200-2,000 mg/m²/day, 200-1,750 mg/m²/day, 200-1,500 mg/m²/day, 200-1,400 mg/m²/day, 200-1,300 mg/m²/day, 200-1,200 mg/m²/day, 200-1,100 mg/m²/day, 200-1,000 mg/m²/day, 200-950 mg/m²/day, 200-900 mg/m²/day, 200-850 mg/m²/day, 200-800 mg/m²/day, 200-750 mg/m²/day, 200-700 mg/m²/day, 200-650 mg/m²/day, 200-600 mg/m²/day, 200-550 mg/m²/day, 200-500 mg/m²/day, 200-450 mg/m²/day, 200-400 mg/m²/day, 200-375 mg/m²/day, 200-350 mg/m²/day, 200-300 mg/m²/day, 200-250 mg/m²/day, 250-2,000 mg/m²/day, 250-1,750 mg/m²/day, 250-1,500 mg/m²/day, 250-1,400 mg/m²/day, 250-1,300 mg/m²/day, 250-1,200 mg/m²/day, 250-1,100 mg/m²/day, 250-1,000 mg/m²/day, 250-950 mg/m²/day, 250-900 mg/m²/day, 250-850 mg/m²/day, 250-800 mg/m²/day, 250-750 mg/m²/day, 250-700 mg/m²/day, 250-650 mg/m²/day, 250-600 mg/m²/day, 250-550 mg/m²/day, 250-500 mg/m²/day, 250-450 mg/m²/day, 250-400 mg/m²/day, 250-375 mg/m²/day, 250-350 mg/m²/day, 250-300 mg/m²/day, 300-2,000 mg/m²/day, 300-1,750 mg/m²/day, 300-1,500 mg/m²/day, 300-1,400 mg/m²/day, 300-1,300 mg/m²/day, 300-1,200 mg/m²/day, 300-1,100 mg/m²/day, 300-1,000 mg/m²/day, 300-950 mg/m²/day, 300-900 mg/m²/day, 300-850 mg/m²/day, 300-800 mg/m²/day, 300-750 mg/m²/day, 300-700 mg/m²/day, 300-650 mg/m²/day, 300-600 mg/m²/day, 300-550 mg/m²/day, 300-500 mg/m²/day, 300-450 mg/m²/day, 300-400 mg/m²/day, 300-375 mg/m²/day, 300-350 mg/m²/day, 350-2,000 mg/m²/day, 350-1,750 mg/m²/day, 350-1,500 mg/m²/day, 350-1,400 mg/m²/day, 350-1,300 mg/m²/day, 350-1,200 mg/m²/day, 350-1,100 mg/m²/day, 350-1,000 mg/m²/day, 350-950 mg/m²/day, 350-900 mg/m²/day, 350-850 mg/m²/day, 350-800 mg/m²/day, 350-750 mg/m²/day, 350-700 mg/m²/day, 350-650 mg/m²/day, 350-600 mg/m²/day, 350-550 mg/m²/day, 350-500 mg/m²/day, 350-450 mg/m²/day, 350-400 mg/m²/day, 350-375 mg/m²/day, 375-2,000 mg/m²/day, 375-1,750 mg/m²/day, 375-1,500 mg/m²/day, 375-1,400 mg/m²/day, 375-1,300 mg/m²/day, 375-1,200 mg/m²/day, 375-1,100 mg/m²/day, 375-1,000 mg/m²/day, 375-950 mg/m²/day, 375-900 mg/m²/day, 375-850 mg/m²/day, 375-800 mg/m²/day, 375-750 mg/m²/day, 375-700 mg/m²/day, 375-650 mg/m²/day, 375-600 mg/m²/day, 375-550 mg/m²/day, 375-500 mg/m²/day, 375-450 mg/m²/day, 375-400 mg/m²/day, 400-2,000 mg/m²/day, 400-1,750 mg/m²/day, 400-1,500 mg/m²/day, 400-1,400 mg/m²/day, 400-1,300 mg/m²/day, 400-1,200 mg/m²/day, 400-1,100 mg/m²/day, 400-1,000 mg/m²/day, 400-950 mg/m²/day, 400-900 mg/m²/day, 400-850 mg/m²/day, 400-800 mg/m²/day, 400-750 mg/m²/day, 400-700 mg/m²/day, 400-650 mg/m²/day, 400-600 mg/m²/day, 400-550 mg/m²/day, 400-500 mg/m²/day, 400-450 mg/m²/day, 450-2,000 mg/m²/day, 450-1,750 mg/m²/day, 450-1,500 mg/m²/day, 450-1,400 mg/m²/day, 450-1,300 mg/m²/day, 450-1,200 mg/m²/day, 450-1,100 mg/m²/day, 450-1,000 mg/m²/day, 450-950 mg/m²/day, 450-900 mg/m²/day, 450-850 mg/m²/day, 450-800 mg/m²/day, 450-750 mg/m²/day, 450-700 mg/m²/day, 450-650 mg/m²/day, 450-600 mg/m²/day, 450-550 mg/m²/day, 450-500 mg/m²/day, 500-2,000 mg/m²/day, 500-1,750 mg/m²/day, 500-1,500 mg/m²/day, 500-1,400 mg/m²/day, 500-1,300 mg/m²/day, 500-1,200 mg/m²/day, 500-1,100 mg/m²/day, 500-1,000 mg/m²/day, 500-950 mg/m²/day, 500-900 mg/m²/day, 500-850 mg/m²/day, 500-800 mg/m²/day, 500-750 mg/m²/day, 500-700 mg/m²/day, 500-650 mg/m²/day, 500-600 mg/m²/day, 500-550 mg/m²/day, 550-2,000 mg/m²/day, 550-1,750 mg/m²/day, 550-1,500 mg/m²/day, 550-1,400 mg/m²/day, 550-1,300 mg/m²/day, 550-1,200 mg/m²/day, 550-1,100 mg/m²/day, 550-1,000 mg/m²/day, 550-950 mg/m²/day, 550-900 mg/m²/day, 550-850 mg/m²/day, 550-800 mg/m²/day, 550-750 mg/m²/day, 550-700 mg/m²/day, 550-650 mg/m²/day, 550-600 mg/m²/day, 600-2,000 mg/m²/day, 600-1,750 mg/m²/day, 600-1,500 mg/m²/day, 600-1,400 mg/m²/day, 600-1,300 mg/m²/day, 600-1,200 mg/m²/day, 600-1,100 mg/m²/day, 600-1,000 mg/m²/day, 600-950 mg/m²/day, 600-900 mg/m²/day, 600-850 mg/m²/day, 600-800 mg/m²/day, 600-750 mg/m²/day, 600-700 mg/m²/day, 600-650 mg/m²/day, 650-2,000 mg/m²/day, 650-1,750 mg/m²/day, 650-1,500 mg/m²/day, 650-1,400 mg/m²/day, 650-1,300 mg/m²/day, 650-1,200 mg/m²/day, 650-1,100 mg/m²/day, 650-1,000 mg/m²/day, 650-950 mg/m²/day, 650-900 mg/m²/day, 650-850 mg/m²/day, 650-800 mg/m²/day, 650-750 mg/m²/day, 650-700 mg/m²/day, 700-2,000 mg/m²/day, 700-1,750 mg/m²/day, 700-1,500 mg/m²/day, 700-1,400 mg/m²/day, 700-1,300 mg/m²/day, 700-1,200 mg/m²/day, 700-1,100 mg/m²/day, 700-1,000 mg/m²/day, 700-950 mg/m²/day, 700-900 mg/m²/day, 700-850 mg/m²/day, 700-800 mg/m²/day, 700-750 mg/m²/day, 750-2,000 mg/m²/day, 750-1,750 mg/m²/day, 750-1,500 mg/m²/day, 750-1,400 mg/m²/day, 750-1,300 mg/m²/day, 750-1,200 mg/m²/day, 750-1,100 mg/m²/day, 750-1,000 mg/m²/day, 750-950 mg/m²/day, 750-900 mg/m²/day, 750-850 mg/m²/day, 750-800 mg/m²/day, 800-2,000 mg/m²/day, 800-1,750 mg/m²/day, 800-1,500 mg/m²/day, 800-1,400 mg/m²/day, 800-1,300 mg/m²/day, 800-1,200 mg/m²/day, 800-1,100 mg/m²/day, 800-1,000 mg/m²/day, 800-950 mg/m²/day, 800-900 mg/m²/day, 800-850 mg/m²/day, 850-2,000 mg/m²/day, 850-1,750 mg/m²/day, 850-1,500 mg/m²/day, 850-1,400 mg/m²/day, 850-1,300 mg/m²/day, 850-1,200 mg/m²/day, 850-1,100 mg/m²/day, 850-1,000 mg/m²/day, 850-950 mg/m²/day, 850-900 mg/m²/day, 900-2,000 mg/m²/day, 900-1,750 mg/m²/day, 900-1,500 mg/m²/day, 900-1,400 mg/m²/day, 900-1,300 mg/m²/day, 900-1,200 mg/m²/day, 900-1,100 mg/m²/day, 900-1,000 mg/m²/day, 900-950 mg/m²/day, 950-2,000 mg/m²/day, 950-1,750 mg/m²/day, 950-1,500 mg/m²/day, 950-1,400 mg/m²/day, 950-1,300 mg/m²/day, 950-1,200 mg/m²/day, 950-1,100 mg/m²/day, 950-1,000 mg/m²/day, 1,000-2,000 mg/m²/day, 1,000-1,750 mg/m²/day, 1,000-1,500 mg/m²/day, 1,000-1,400 mg/m²/day, 1,000-1,300 mg/m²/day, 1,000-1,200 mg/m²/day, 1,000-1,100 mg/m²/day, 1,100-2,000 mg/m²/day, 1,100-1,750 mg/m²/day, 1,100-1,500 mg/m²/day, 1,100-1,400 mg/m²/day, 1,100-1,300 mg/m²/day, 1,100-1,200 mg/m²/day, 1,200-2,000 mg/m²/day, 1,200-1,750 mg/m²/day, 1,200-1,500 mg/m²/day, 1,200-1,400 mg/m²/day, 1,200-1,300 mg/m²/day, 1,300-2,000 mg/m²/day, 1,300-1,750 mg/m²/day, 1,300-1,500 mg/m²/day, 1,300-1,400 mg/m²/day, 1,400-2,000 mg/m²/day, 1,400-1,750 mg/m²/day, 1,400-1,500 mg/m²/day, 1,500-2,000 mg/m²/day, 1,500-1,750 mg/m²/day, 1,600-2,000 mg/m²/day, 1,600-1,750 mg/m²/day, or 1,750-2,000 mg/m²/day cyclophosphamide to the subject. In certain embodiments, the dose of cyclophosphamide is 200-1,500 mg/m²/day, for example, 250-1,000 mg/m²/day or 500-1,000 mg/m²/day.

In certain embodiments, a preconditioning regimen of the disclosure comprises administering a dose of cyclophosphamide and a dose of fludarabine to the subject, wherein the dose of cyclophosphamide is less than 100 mg/m²/day, less than 125 mg/m²/day, less than 150 mg/m²/day, less than 175 mg/m²/day, less than 200 mg/m²/day, less than 225 mg/m²/day, less than 250 mg/m²/day, less than 275 mg/m²/day, less than 300 mg/m²/day, less than 350 mg/m²/day, less than 375 mg/m²/day, less than 400 mg/m²/day, less than 450 mg/m²/day, less than 500 mg/m²/day, less than 550 mg/m²/day, less than 600 mg/m²/day, less than 650 mg/m²/day, less than 700 mg/m²/day, less than 750 mg/m²/day, less than 800 mg/m²/day, less than 850 mg/m²/day, less than 900 mg/m²/day, less than 950 mg/m²/day, less than 1,000 mg/m²/day, less than 1,100 mg/m²/day, less than 1,200 mg/m²/day, less than 1,250 mg/m²/day, less than 1,300 mg/m²/day, less than 1,400 mg/m²/day, less than 1,500 mg/m²/day, less than 1,600 mg/m²/day, less than 1,700 mg/m²/day, less than 1,750 mg/m²/day, less than 1,800 mg/m²/day, less than 1,900 mg/m²/day, or less than 2,000 mg/m²/day. In certain embodiments, the dose of cyclophosphamide administered to the subject is less than 1,000 mg/m²/day. In certain embodiments, the dose of cyclophosphamide administered to the subject is less than 600 mg/m²/day. In certain embodiments, the dose of cyclophosphamide administered to the subject is less than 500 mg/m²/day.

In certain embodiments, a preconditioning regimen of the disclosure comprises administering a dose of cyclophosphamide and a dose of fludarabine to the subject, wherein the dose of cyclophosphamide is 100 mg/m²/day, 125 mg/m²/day, 150 mg/m²/day, 175 mg/m²/day, 200 mg/m²/day, 225 mg/m²/day, 250 mg/m²/day, 275 mg/m²/day, 300 mg/m²/day, 350 mg/m²/day, 375 mg/m²/day, 400 mg/m²/day, 450 mg/m²/day, 500 mg/m²/day, 550 mg/m²/day, 600 mg/m²/day, 650 mg/m²/day, 700 mg/m²/day, 750 mg/m²/day, 800 mg/m²/day, 850 mg/m²/day, 900 mg/m²/day, 950 mg/m²/day, 1,000 mg/m²/day, 1,100 mg/m²/day, 1,200 mg/m²/day, 1,250 mg/m²/day, 1,300 mg/m²/day, 1,400 mg/m²/day, 1,500 mg/m²/day, 1,600 mg/m²/day, 1,700 mg/m²/day, 1,750 mg/m²/day, 1,800 mg/m²/day, 1,900 mg/m²/day, or 2,000 mg/m²/day cyclophosphamide to the subject. In certain embodiments, the dose of cyclophosphamide is 250 mg/m²/day. In certain embodiments, the dose of cyclophosphamide is 500 mg/m²/day. In certain embodiments, the dose of cyclophosphamide is 1,000 mg/m²/day.

In certain embodiments, a preconditioning regimen of the disclosure comprises administering a dose of cyclophosphamide and a dose of fludarabine to the subject, wherein the dose of cyclophosphamide is administered to the subject daily. In certain embodiments, the dose of cyclophosphamide is administered to the subject daily for 1, 2, 3, 4, or 5 days. In certain embodiments, the dose of cyclophosphamide is administered to the subject daily for 2 days. In certain embodiments, the dose of cyclophosphamide is administered to the subject on a single day.

In certain embodiments, a therapeutic method of the disclosure comprises (a) administering to the subject a preconditioning regimen comprising a dose of cyclophosphamide and a dose of fludarabine; and (b) after step (a), administering to the subject a therapeutically effective amount of engineered immune cells (e.g., CAR immune cells, e.g., CAR T cells).

In certain embodiments, the subject receives the cyclophosphamide no less than 1 day, 2 days, 3 days, 4 days, 5 days, 6 days, 7 days, 8 days, 9 days, or 10 days prior to step (b) (e.g., prior to initiation of step (b)). In certain embodiments, the subject receives the cyclophosphamide no less than 1 day, 2 days, 3 days, 4 days, or 5 days prior to step (b) (e.g., prior to initiation of step (b)). In certain embodiments, the subject receives the cyclophosphamide no less than 1 day, 2 days, or 3 days prior to step (b) (e.g., prior to initiation of step (b)). In certain embodiments, the subject receives the cyclophosphamide no less than 3 days prior to step (b) (e.g., prior to initiation of step (b)).

In certain embodiments, the dose of cyclophosphamide is administered to the subject in the 10 days prior to step (b) (e.g., prior to initiation of step (b)). In certain embodiments, the dose of cyclophosphamide is administered to the subject in the 7 days prior to step (b) (e.g., prior to initiation of step (b)). In certain embodiments, the dose of cyclophosphamide is administered to the subject in the 5 days prior to step (b) (e.g., prior to initiation of step (b)). In certain embodiments, the dose of cyclophosphamide is administered to the subject in the 4 days prior to step (b) (e.g., prior to initiation of step (b)). In certain embodiments, the dose of cyclophosphamide is administered to the subject in the 3 days prior to step (b) (e.g., prior to initiation of step (b)).

In certain embodiments, the dose of cyclophosphamide is administered to the subject 10 days, 9 days, 8 days, 7 days, 6 days, 5 days, 4 days, 3 days, 2 days, and/or 1 day prior to step (b) (e.g., prior to initiation of step (b)). In certain embodiments, the dose of cyclophosphamide is administered to the subject 7 days, 6 days, 5 days, 4 days, 3 days, 2 days, and/or 1 day prior to step (b) (e.g., prior to initiation of step (b)). In certain embodiments, the dose of cyclophosphamide is administered to the subject 5 days, 4 days, 3 days, 2 days, and/or 1 day prior to step (b) (e.g., prior to initiation of step (b)). In certain embodiments, the dose of cyclophosphamide is administered to the subject 2 days prior to step (b) (e.g., prior to initiation of step (b)). In certain embodiments, the dose of cyclophosphamide is administered to the subject 1 day prior to step (b) (e.g., prior to initiation of step (b)). In certain embodiments, the dose of cyclophosphamide is administered to the subject 4 days prior to step (b) (e.g., prior to initiation of step (b)). In certain embodiments, the dose of cyclophosphamide is administered to the subject 3 days prior to step (b) (e.g., prior to initiation of step (b)). In certain embodiments, the dose of cyclophosphamide is administered to the subject 4 days and 3 days prior to step (b) (e.g., prior to initiation of step (b)).

In certain embodiments, step (b) is defined as occurring on “Day 0”, and “Day −X” and “Day X” refer to the day occurring X days prior to or after Day 0, respectively. In certain embodiments, the dose of cyclophosphamide is administered to the subject on or prior to Day −1, Day −2, Day −3, Day −4, Day −5, Day −6, Day −7, Day −8, Day −9, or Day −10. In certain embodiments, the dose of cyclophosphamide is administered to the subject on or prior to Day −2. In certain embodiments, the dose of cyclophosphamide is administered to the subject on or prior to Day −3.

In certain embodiments, the dose of cyclophosphamide is administered to the subject on or after Day −10, and before Day 0. In certain embodiments, the dose of cyclophosphamide is administered to the subject on or after Day −10, and before Day −2. In certain embodiments, the dose of cyclophosphamide is administered to the subject on or after Day −7, and before Day 0. In certain embodiments, the dose of cyclophosphamide is administered to the subject on or after Day −7, and before Day −2. In certain embodiments, the dose of cyclophosphamide is administered to the subject on or after Day −6, and before Day 0. In certain embodiments, the dose of cyclophosphamide is administered to the subject on or after Day −6, and before Day −2. In certain embodiments, the dose of cyclophosphamide is administered to the subject on or after Day −5, and before Day 0. In certain embodiments, the dose of cyclophosphamide is administered to the subject on or after Day −5, and before Day −2. In certain embodiments, the dose of cyclophosphamide is administered to the subject on or after Day −4, and before Day 0. In certain embodiments, the one or more doses of cyclophosphamide are administered to the subject on or after Day −4, and before Day −2. In certain embodiments, the one or more doses of cyclophosphamide are administered to the subject on or after Day −3, and before Day 0.

In certain embodiments, the preconditioning regimen comprises administering a dose of cyclophosphamide to the subject on Day −10, Day −9, Day −8, Day −7, Day −6, Day −5, Day −4, Day −3, Day −2, and/or Day −1. In certain embodiments, the preconditioning regimen comprises administering a dose of cyclophosphamide to the subject on Day −7, Day −6, Day −5, Day −4, Day −3, Day −2, and/or Day −1. In certain embodiments, the preconditioning regimen comprises administering a dose of cyclophosphamide to the subject on Day −5, Day −4, Day −3, Day −2, and/or Day −1. In certain embodiments, the preconditioning regimen comprises administering a dose of cyclophosphamide to the subject on Day −2. In certain embodiments, the preconditioning regimen comprises administering a dose of cyclophosphamide to the subject on Day −1. In certain embodiments, the preconditioning regimen comprises administering a dose of cyclophosphamide to the subject on Day −4. In certain embodiments, the preconditioning regimen comprises administering a dose of cyclophosphamide to the subject on Day −3. In certain embodiments, the preconditioning regimen comprises administering a dose of cyclophosphamide to the subject on Day −4 and Day −3.

In certain embodiments, a preconditioning regimen of the disclosure comprises administering a dose of cyclophosphamide and a dose of fludarabine to the subject, wherein the total dose of cyclophosphamide administered to the subject is 100-2,000 mg/m². If, for example, a subject is administered a dose of 500 mg/m²/day cyclophosphamide on Day −4 and Day −3, then the subject is considered to have been administered a total dose of 1,000 mg/m² cyclophosphamide. If a subject is administered a single dose of 1,000 mg/m²/day cyclophosphamide on Day −3, then the subject is considered to have been administered a total dose of 1,000 mg/m² cyclophosphamide. In certain embodiments, the preconditioning regimen comprises administering a total dose of 100-2,000 mg/m², 100-1,750 mg/m², 100-1,500 mg/m², 100-1,400 mg/m², 100-1,300 mg/m², 100-1,200 mg/m², 100-1,100 mg/m², 100-1,000 mg/m², 100-950 mg/m², 100-900 mg/m², 100-850 mg/m², 100-800 mg/m², 100-750 mg/m², 100-700 mg/m², 100-650 mg/m², 100-600 mg/m², 100-550 mg/m², 100-500 mg/m², 100-450 mg/m², 100-400 mg/m², 100-375 mg/m², 100-350 mg/m², 100-300 mg/m², 100-250 mg/m², 100-200 mg/m², 100-150 mg/m², 150-2,000 mg/m², 150-1,750 mg/m², 150-1,500 mg/m², 150-1,400 mg/m², 150-1,300 mg/m², 150-1,200 mg/m², 150-1,100 mg/m², 150-1,000 mg/m², 150-950 mg/m², 150-900 mg/m², 150-850 mg/m², 150-800 mg/m², 150-750 mg/m², 150-700 mg/m², 150-650 mg/m², 150-600 mg/m², 150-550 mg/m², 150-500 mg/m², 150-450 mg/m², 150-400 mg/m², 150-375 mg/m², 150-350 mg/m², 150-300 mg/m², 150-250 mg/m², 150-200 mg/m², 200-2,000 mg/m², 200-1,750 mg/m², 200-1,500 mg/m², 200-1,400 mg/m², 200-1,300 mg/m², 200-1,200 mg/m², 200-1,100 mg/m², 200-1,000 mg/m², 200-950 mg/m², 200-900 mg/m², 200-850 mg/m², 200-800 mg/m², 200-750 mg/m², 200-700 mg/m², 200-650 mg/m², 200-600 mg/m², 200-550 mg/m², 200-500 mg/m², 200-450 mg/m², 200-400 mg/m², 200-375 mg/m², 200-350 mg/m², 200-300 mg/m², 200-250 mg/m², 250-2,000 mg/m², 250-1,750 mg/m², 250-1,500 mg/m², 250-1,400 mg/m², 250-1,300 mg/m², 250-1,200 mg/m², 250-1,100 mg/m², 250-1,000 mg/m², 250-950 mg/m², 250-900 mg/m², 250-850 mg/m², 250-800 mg/m², 250-750 mg/m², 250-700 mg/m², 250-650 mg/m², 250-600 mg/m², 250-550 mg/m², 250-500 mg/m², 250-450 mg/m², 250-400 mg/m², 250-375 mg/m², 250-350 mg/m², 250-300 mg/m², 300-2,000 mg/m², 300-1,750 mg/m², 300-1,500 mg/m², 300-1,400 mg/m², 300-1,300 mg/m², 300-1,200 mg/m², 300-1,100 mg/m², 300-1,000 mg/m², 300-950 mg/m², 300-900 mg/m², 300-850 mg/m², 300-800 mg/m², 300-750 mg/m², 300-700 mg/m², 300-650 mg/m², 300-600 mg/m², 300-550 mg/m², 300-500 mg/m², 300-450 mg/m², 300-400 mg/m², 300-375 mg/m², 300-350 mg/m², 350-2,000 mg/m², 350-1,750 mg/m², 350-1,500 mg/m², 350-1,400 mg/m², 350-1,300 mg/m², 350-1,200 mg/m², 350-1,100 mg/m², 350-1,000 mg/m², 350-950 mg/m², 350-900 mg/m², 350-850 mg/m², 350-800 mg/m², 350-750 mg/m², 350-700 mg/m², 350-650 mg/m², 350-600 mg/m², 350-550 mg/m², 350-500 mg/m², 350-450 mg/m², 350-400 mg/m², 350-375 mg/m², 375-2,000 mg/m², 375-1,750 mg/m², 375-1,500 mg/m², 375-1,400 mg/m², 375-1,300 mg/m², 375-1,200 mg/m², 375-1,100 mg/m², 375-1,000 mg/m², 375-950 mg/m², 375-900 mg/m², 375-850 mg/m², 375-800 mg/m², 375-750 mg/m², 375-700 mg/m², 375-650 mg/m², 375-600 mg/m², 375-550 mg/m², 375-500 mg/m², 375-450 mg/m², 375-400 mg/m², 400-2,000 mg/m², 400-1,750 mg/m², 400-1,500 mg/m², 400-1,400 mg/m², 400-1,300 mg/m², 400-1,200 mg/m², 400-1,100 mg/m², 400-1,000 mg/m², 400-950 mg/m², 400-900 mg/m², 400-850 mg/m², 400-800 mg/m², 400-750 mg/m², 400-700 mg/m², 400-650 mg/m², 400-600 mg/m², 400-550 mg/m², 400-500 mg/m², 400-450 mg/m², 450-2,000 mg/m², 450-1,750 mg/m², 450-1,500 mg/m², 450-1,400 mg/m², 450-1,300 mg/m², 450-1,200 mg/m², 450-1,100 mg/m², 450-1,000 mg/m², 450-950 mg/m², 450-900 mg/m², 450-850 mg/m², 450-800 mg/m², 450-750 mg/m², 450-700 mg/m², 450-650 mg/m², 450-600 mg/m², 450-550 mg/m², 450-500 mg/m², 500-2,000 mg/m², 500-1,750 mg/m², 500-1,500 mg/m², 500-1,400 mg/m², 500-1,300 mg/m², 500-1,200 mg/m², 500-1,100 mg/m², 500-1,000 mg/m², 500-950 mg/m², 500-900 mg/m², 500-850 mg/m², 500-800 mg/m², 500-750 mg/m², 500-700 mg/m², 500-650 mg/m², 500-600 mg/m², 500-550 mg/m², 550-2,000 mg/m², 550-1,750 mg/m², 550-1,500 mg/m², 550-1,400 mg/m², 550-1,300 mg/m², 550-1,200 mg/m², 550-1,100 mg/m², 550-1,000 mg/m², 550-950 mg/m², 550-900 mg/m², 550-850 mg/m², 550-800 mg/m², 550-750 mg/m², 550-700 mg/m², 550-650 mg/m², 550-600 mg/m², 600-2,000 mg/m², 600-1,750 mg/m², 600-1,500 mg/m², 600-1,400 mg/m², 600-1,300 mg/m², 600-1,200 mg/m², 600-1,100 mg/m², 600-1,000 mg/m², 600-950 mg/m², 600-900 mg/m², 600-850 mg/m², 600-800 mg/m², 600-750 mg/m², 600-700 mg/m², 600-650 mg/m², 650-2,000 mg/m², 650-1,750 mg/m², 650-1,500 mg/m², 650-1,400 mg/m², 650-1,300 mg/m², 650-1,200 mg/m², 650-1,100 mg/m², 650-1,000 mg/m², 650-950 mg/m², 650-900 mg/m², 650-850 mg/m², 650-800 mg/m², 650-750 mg/m², 650-700 mg/m², 700-2,000 mg/m², 700-1,750 mg/m², 700-1,500 mg/m², 700-1,400 mg/m², 700-1,300 mg/m², 700-1,200 mg/m², 700-1,100 mg/m², 700-1,000 mg/m², 700-950 mg/m², 700-900 mg/m², 700-850 mg/m², 700-800 mg/m², 700-750 mg/m², 750-2,000 mg/m², 750-1,750 mg/m², 750-1,500 mg/m², 750-1,400 mg/m², 750-1,300 mg/m², 750-1,200 mg/m², 750-1,100 mg/m², 750-1,000 mg/m², 750-950 mg/m², 750-900 mg/m², 750-850 mg/m², 750-800 mg/m², 800-2,000 mg/m², 800-1,750 mg/m², 800-1,500 mg/m², 800-1,400 mg/m², 800-1,300 mg/m², 800-1,200 mg/m², 800-1,100 mg/m², 800-1,000 mg/m², 800-950 mg/m², 800-900 mg/m², 800-850 mg/m², 850-2,000 mg/m², 850-1,750 mg/m², 850-1,500 mg/m², 850-1,400 mg/m², 850-1,300 mg/m², 850-1,200 mg/m², 850-1,100 mg/m², 850-1,000 mg/m², 850-950 mg/m², 850-900 mg/m², 900-2,000 mg/m², 900-1,750 mg/m², 900-1,500 mg/m², 900-1,400 mg/m², 900-1,300 mg/m², 900-1,200 mg/m², 900-1,100 mg/m², 900-1,000 mg/m², 900-950 mg/m², 950-2,000 mg/m², 950-1,750 mg/m², 950-1,500 mg/m², 950-1,400 mg/m², 950-1,300 mg/m², 950-1,200 mg/m², 950-1,100 mg/m², 950-1,000 mg/m², 1,000-2,000 mg/m², 1,000-1,750 mg/m², 1,000-1,500 mg/m², 1,000-1,400 mg/m², 1,000-1,300 mg/m², 1,000-1,200 mg/m², 1,000-1,100 mg/m², 1,100-2,000 mg/m², 1,100-1,750 mg/m², 1,100-1,500 mg/m², 1,100-1,400 mg/m², 1,100-1,300 mg/m², 1,100-1,200 mg/m², 1,200-2,000 mg/m², 1,200-1,750 mg/m², 1,200-1,500 mg/m², 1,200-1,400 mg/m², 1,200-1,300 mg/m², 1,300-2,000 mg/m², 1,300-1,750 mg/m², 1,300-1,500 mg/m², 1,300-1,400 mg/m², 1,400-2,000 mg/m², 1,400-1,750 mg/m², 1,400-1,500 mg/m², 1,500-2,000 mg/m², 1,500-1,750 mg/m², or 1,750-2,000 mg/m² cyclophosphamide to the subject. In certain embodiments, the total dose of cyclophosphamide is administered to the subject in a single day.

In certain embodiments, a preconditioning regimen of the disclosure comprises administering a dose of cyclophosphamide and a dose of fludarabine to the subject, wherein the total dose of cyclophosphamide administered to the subject is less than 2,000 mg/m². For example, in certain embodiments, the total dose of cyclophosphamide administered to the subject is less than 100 mg/m², less than 125 mg/m², less than 150 mg/m², less than 175 mg/m², less than 200 mg/m², less than 225 mg/m², less than 250 mg/m², less than 275 mg/m², less than 300 mg/m², less than 350 mg/m², less than 375 mg/m², less than 400 mg/m², less than 450 mg/m², less than 500 mg/m², less than 550 mg/m², less than 600 mg/m², less than 650 mg/m², less than 700 mg/m², less than 750 mg/m², less than 800 mg/m², less than 850 mg/m², less than 900 mg/m², less than 950 mg/m², less than 1,000 mg/m², less than 1,100 mg/m², less than 1,200 mg/m², less than 1,250 mg/m², less than 1,300 mg/m², less than 1,400 mg/m², less than 1,500 mg/m², less than 1,600 mg/m², less than 1,700 mg/m², less than 1,750 mg/m², less than 1,800 mg/m², less than 1,900 mg/m², or less than 2,000 mg/m². In certain embodiments, the total dose of cyclophosphamide administered to the subject is less than 2,000 mg/m². In certain embodiments, the total dose of cyclophosphamide administered to the subject is less than 1,000 mg/m². In certain embodiments, the total dose of cyclophosphamide administered to the subject is less than 800 mg/m². In certain embodiments, the total dose of cyclophosphamide administered to the subject is less than 600 mg/m². In certain embodiments, the total dose of cyclophosphamide administered to the subject is less than 500 mg/m².

In certain embodiments, a preconditioning regimen of the disclosure comprises administering a dose of cyclophosphamide and a dose of fludarabine to the subject, wherein the total dose of cyclophosphamide administered to the subject is 100 mg/m², 125 mg/m², 150 mg/m², 175 mg/m², 200 mg/m², 225 mg/m², 250 mg/m², 275 mg/m², 300 mg/m², 350 mg/m², 375 mg/m², 400 mg/m², 450 mg/m², 500 mg/m², 550 mg/m², 600 mg/m², 650 mg/m², 700 mg/m², 750 mg/m², 800 mg/m², 850 mg/m², 900 mg/m², 950 mg/m², 1,000 mg/m², 1,100 mg/m², 1,200 mg/m², 1,250 mg/m², 1,300 mg/m², 1,400 mg/m², 1,500 mg/m², 1,600 mg/m², 1,700 mg/m², 1,750 mg/m², 1,800 mg/m², 1,900 mg/m², or 2,000 mg/m². In certain embodiments, the total dose of cyclophosphamide administered to the subject is 500 mg/m². In certain embodiments, the total dose of cyclophosphamide administered to the subject is 1,000 mg/m².

In certain embodiments, a preconditioning regimen of the disclosure comprises administering to the subject a dose of cyclophosphamide and a dose of fludarabine, wherein the dose of fludarabine is 2-20 mg/m²/day. For example, in certain embodiments, the preconditioning regimen comprises administering a dose of 2-20 mg/m²/day, 2-19 mg/m²/day, 2-18 mg/m²/day, 2-17 mg/m²/day, 2-16 mg/m²/day, 2-15 mg/m²/day, 2-14 mg/m²/day, 2-13 mg/m²/day, 2-12.5 mg/m²/day, 2-12 mg/m²/day, 2-11 mg/m²/day, 2-10 mg/m²/day, 2-9 mg/m²/day, 2-8 mg/m²/day, 2-7.5 mg/m²/day, 2-7 mg/m²/day, 2-6.5 mg/m²/day, 2-6.25 mg/m²/day, 2-6 mg/m²/day, 2-5 mg/m²/day, 2-4 mg/m²/day, 2-3 mg/m²/day, 3-20 mg/m²/day, 3-19 mg/m²/day, 3-18 mg/m²/day, 3-17 mg/m²/day, 3-16 mg/m²/day, 3-15 mg/m²/day, 3-14 mg/m²/day, 3-13 mg/m²/day, 3-12.5 mg/m²/day, 3-12 mg/m²/day, 3-11 mg/m²/day, 3-10 mg/m²/day, 3-9 mg/m²/day, 3-8 mg/m²/day, 3-7.5 mg/m²/day, 3-7 mg/m²/day, 3-6.5 mg/m²/day, 3-6.25 mg/m²/day, 3-6 mg/m²/day, 3-5 mg/m²/day, 3-4 mg/m²/day, 4-20 mg/m²/day, 4-19 mg/m²/day, 4-18 mg/m²/day, 4-17 mg/m²/day, 4-16 mg/m²/day, 4-15 mg/m²/day, 4-14 mg/m²/day, 4-13 mg/m²/day, 4-12.5 mg/m²/day, 4-12 mg/m²/day, 4-11 mg/m²/day, 4-10 mg/m²/day, 4-9 mg/m²/day, 4-8 mg/m²/day, 4-7.5 mg/m²/day, 4-7 mg/m²/day, 4-6.5 mg/m²/day, 4-6.25 mg/m²/day, 4-6 mg/m²/day, 4-5 mg/m²/day, 5-20 mg/m²/day, 5-19 mg/m²/day, 5-18 mg/m²/day, 5-17 mg/m²/day, 5-16 mg/m²/day, 5-15 mg/m²/day, 5-14 mg/m²/day, 5-13 mg/m²/day, 5-12.5 mg/m²/day, 5-12 mg/m²/day, 5-11 mg/m²/day, 5-10 mg/m²/day, 5-9 mg/m²/day, 5-8 mg/m²/day, 5-7.5 mg/m²/day, 5-7 mg/m²/day, 5-6.5 mg/m²/day, 5-6.25 mg/m²/day, 5-6 mg/m²/day, 6-20 mg/m²/day, 6-19 mg/m²/day, 6-18 mg/m²/day, 6-17 mg/m²/day, 6-16 mg/m²/day, 6-15 mg/m²/day, 6-14 mg/m²/day, 6-13 mg/m²/day, 6-12.5 mg/m²/day, 6-12 mg/m²/day, 6-11 mg/m²/day, 6-10 mg/m²/day, 6-9 mg/m²/day, 6-8 mg/m²/day, 6-7.5 mg/m²/day, 6-7 mg/m²/day, 6-6.5 mg/m²/day, 6-6.25 mg/m²/day, 6.25-20 mg/m²/day, 6.25-19 mg/m²/day, 6.25-18 mg/m²/day, 6.25-17 mg/m²/day, 6.25-16 mg/m²/day, 6.25-15 mg/m²/day, 6.25-14 mg/m²/day, 6.25-13 mg/m²/day, 6.25-12.5 mg/m²/day, 6.25-12 mg/m²/day, 6.25-11 mg/m²/day, 6.25-10 mg/m²/day, 6.25-9 mg/m²/day, 6.25-8 mg/m²/day, 6.25-7.5 mg/m²/day, 6.25-7 mg/m²/day, 6.25-6.5 mg/m²/day, 6.5-20 mg/m²/day, 6.5-19 mg/m²/day, 6.5-18 mg/m²/day, 6.5-17 mg/m²/day, 6.5-16 mg/m²/day, 6.5-15 mg/m²/day, 6.5-14 mg/m²/day, 6.5-13 mg/m²/day, 6.5-12.5 mg/m²/day, 6.5-12 mg/m²/day, 6.5-11 mg/m²/day, 6.5-10 mg/m²/day, 6.5-9 mg/m²/day, 6.5-8 mg/m²/day, 6.5-7.5 mg/m²/day, 6.5-7 mg/m²/day, 7-20 mg/m²/day, 7-19 mg/m²/day, 7-18 mg/m²/day, 7-17 mg/m²/day, 7-16 mg/m²/day, 7-15 mg/m²/day, 7-14 mg/m²/day, 7-13 mg/m²/day, 7-12.5 mg/m²/day, 7-12 mg/m²/day, 7-11 mg/m²/day, 7-10 mg/m²/day, 7-9 mg/m²/day, 7-8 mg/m²/day, 7-7.5 mg/m²/day, 7.5-20 mg/m²/day, 7.5-19 mg/m²/day, 7.5-18 mg/m²/day, 7.5-17 mg/m²/day, 7.5-16 mg/m²/day, 7.5-15 mg/m²/day, 7.5-14 mg/m²/day, 7.5-13 mg/m²/day, 7.5-12.5 mg/m²/day, 7.5-12 mg/m²/day, 7.5-11 mg/m²/day, 7.5-10 mg/m²/day, 7.5-9 mg/m²/day, 7.5-8 mg/m²/day, 8-20 mg/m²/day, 8-19 mg/m²/day, 8-18 mg/m²/day, 8-17 mg/m²/day, 8-16 mg/m²/day, 8-15 mg/m²/day, 8-14 mg/m²/day, 8-13 mg/m²/day, 8-12.5 mg/m²/day, 8-12 mg/m²/day, 8-11 mg/m²/day, 8-10 mg/m²/day, 8-9 mg/m²/day, 9-20 mg/m²/day, 9-19 mg/m²/day, 9-18 mg/m²/day, 9-17 mg/m²/day, 9-16 mg/m²/day, 9-15 mg/m²/day, 9-14 mg/m²/day, 9-13 mg/m²/day, 9-12.5 mg/m²/day, 9-12 mg/m²/day, 9-11 mg/m²/day, 9-10 mg/m²/day, 10-20 mg/m²/day, 10-19 mg/m²/day, 10-18 mg/m²/day, 10-17 mg/m²/day, 10-16 mg/m²/day, 10-15 mg/m²/day, 10-14 mg/m²/day, 10-13 mg/m²/day, 10-12.5 mg/m²/day, 10-12 mg/m²/day, 10-11 mg/m²/day, 11-20 mg/m²/day, 11-19 mg/m²/day, 11-18 mg/m²/day, 11-17 mg/m²/day, 11-16 mg/m²/day, 11-15 mg/m²/day, 11-14 mg/m²/day, 11-13 mg/m²/day, 11-12.5 mg/m²/day, 11-12 mg/m²/day, 12-20 mg/m²/day, 12-19 mg/m²/day, 12-18 mg/m²/day, 12-17 mg/m²/day, 12-16 mg/m²/day, 12-15 mg/m²/day, 12-14 mg/m²/day, 12-13 mg/m²/day, 12-12.5 mg/m²/day, 12.5-20 mg/m²/day, 12.5-19 mg/m²/day, 12.5-18 mg/m²/day, 12.5-17 mg/m²/day, 12.5-16 mg/m²/day, 12.5-15 mg/m²/day, 12.5-14 mg/m²/day, 12.5-13 mg/m²/day, 13-20 mg/m²/day, 13-19 mg/m²/day, 13-18 mg/m²/day, 13-17 mg/m²/day, 13-16 mg/m²/day, 13-15 mg/m²/day, 13-14 mg/m²/day, 14-20 mg/m²/day, 14-19 mg/m²/day, 14-18 mg/m²/day, 14-17 mg/m²/day, 14-16 mg/m²/day, 14-15 mg/m²/day, 15-20 mg/m²/day, 15-19 mg/m²/day, 15-18 mg/m²/day, 15-17 mg/m²/day, 15-16 mg/m²/day, 16-20 mg/m²/day, 16-19 mg/m²/day, 16-18 mg/m²/day, 16-17 mg/m²/day, 17-20 mg/m²/day, 17-19 mg/m²/day, 17-18 mg/m²/day, 18-20 mg/m²/day, 18-19 mg/m²/day, or 19-20 mg/m²/day fludarabine to the subject. In certain embodiments, the dose of fludarabine is 5-15 mg/m²/day. In certain embodiments, the dose of fludarabine is 5-12.5 mg/m²/day. In certain embodiments, the dose of fludarabine is 5-10 mg/m²/day. In certain embodiments, the dose of fludarabine is 5-6.25 mg/m²/day. In certain embodiments, the dose of fludarabine is 6.25-15 mg/m²/day. In certain embodiments, the dose of fludarabine is 6.25-12.5 mg/m²/day. In certain embodiments, the dose of fludarabine is 6.25-10 mg/m²/day. In certain embodiments, dose of fludarabine is 10-15 mg/m²/day. In certain embodiments, dose of fludarabine is 10-12.5 mg/m²/day. In certain embodiments, the dose of fludarabine is 12.5-15 mg/m²/day.

In certain embodiments, a preconditioning regimen of the disclosure comprises administering a dose of cyclophosphamide and a dose of fludarabine to the subject, wherein the dose of fludarabine is less than 2 mg/m²/day, less than 3 mg/m²/day, less than 4 mg/m²/day, less than 5 mg/m²/day, less than 6 mg/m²/day, less than 6.25 mg/m²/day, less than 6.5 mg/m²/day, less than 7 mg/m²/day, less than 7.5 mg/m²/day, less than 8 mg/m²/day, less than 9 mg/m²/day, less than 10 mg/m²/day, less than 11 mg/m²/day, less than 12 mg/m²/day, less than 12.5 mg/m²/day, less than 13 mg/m²/day, less than 14 mg/m²/day, less than 15 mg/m²/day, less than 16 mg/m²/day, less than 17 mg/m²/day, less than 18 mg/m²/day, less than 19 mg/m²/day, less than 20 mg/m²/day, less than 22.5 mg/m²/day, or less than 25 mg/m²/day. In certain embodiments, the dose of fludarabine is less than 20 mg/m²/day. In certain embodiments, the dose of fludarabine is less than 15 mg/m²/day. In certain embodiments, the dose of fludarabine is less than 12.5 mg/m²/day. In certain embodiments, the dose of fludarabine is less than 10 mg/m²/day.

In certain embodiments, a preconditioning regimen of the disclosure comprises administering a dose of cyclophosphamide and a dose of fludarabine to the subject, wherein the dose of fludarabine is 2 mg/m²/day, 3 mg/m²/day, 4 mg/m²/day, 5 mg/m²/day, 6 mg/m²/day, 6.25 mg/m²/day, 6.5 mg/m²/day, 7 mg/m²/day, 7.5 mg/m²/day, 8 mg/m²/day, 9 mg/m²/day, 10 mg/m²/day, 11 mg/m²/day, 12 mg/m²/day, 12.5 mg/m²/day, 13 mg/m²/day, 14 mg/m²/day, 15 mg/m²/day, 16 mg/m²/day, 17 mg/m²/day, 18 mg/m²/day, 19 mg/m²/day, 20 mg/m²/day, 22.5 mg/m²/day, or 25 mg/m²/day. In certain embodiments, the dose of fludarabine is 15 mg/m²/day, for example, 12.5 mg/m²/day or 6.25 mg/m²/day.

In certain embodiments, a preconditioning regimen of the disclosure comprises administering a dose of cyclophosphamide and a dose of fludarabine to the subject, wherein the dose of fludarabine is administered to the subject daily. In certain embodiments, the dose of fludarabine is administered to the subject daily for 1, 2, 3, 4, or 5 days. In certain embodiments, the dose of fludarabine is administered to the subject daily for 3 days.

In certain embodiments, a therapeutic method of the disclosure comprises (a) administering to the subject a preconditioning regimen comprising a dose of cyclophosphamide and a dose of fludarabine; and (b) after step (a), administering to the subject a therapeutically effective amount of engineered immune cells (e.g., CAR immune cells, e.g., CAR T cells).

In certain embodiments, the subject receives the fludarabine no less than 1 day, 2 days, 3 days, 4 days, 5 days, 6 days, 7 days, 8 days, 9 days, or 10 days prior to step (b) (e.g., prior to initiation of step (b)). In certain embodiments, the subject receives the fludarabine no less than 1 day, 2 days, 3 days, 4 days, or 5 days prior to step (b) (e.g., prior to initiation of step (b)). In certain embodiments, the subject receives the fludarabine no less than 1 day, 2 days, or 3 days prior to step (b) (e.g., prior to initiation of step (b)). In certain embodiments, the subject receives the fludarabine no less than 3 days prior to step (b) (e.g., prior to initiation of step (b)).

In certain embodiments, the dose of fludarabine is administered to the subject in the 10 days prior to step (b) (e.g., prior to initiation of step (b)). In certain embodiments, the dose of fludarabine is administered to the subject in the 7 days prior to step (b) (e.g., prior to initiation of step (b)). In certain embodiments, the dose of fludarabine is administered to the subject in the 5 days prior to step (b) (e.g., prior to initiation of step (b)). In certain embodiments, the dose of fludarabine is administered to the subject 10 days, 9 days, 8 days, 7 days, 6 days, 5 days, 4 days, 3 days, 2 days, and/or 1 day prior to step (b) (e.g., prior to initiation of step (b)). In certain embodiments, the dose of fludarabine is administered to the subject 7 days, 6 days, 5 days, 4 days, 3 days, 2 days, and/or 1 day prior to step (b) (e.g., prior to initiation of step (b)). In certain embodiments, the dose of fludarabine is administered to the subject 5 days, 4 days, 3 days, 2 days, and/or 1 day prior to step (b) (e.g., prior to initiation of step (b)). In certain embodiments, the dose of fludarabine is administered to the subject 5 days, 4 days, and/or 3 days prior to step (b) (e.g., prior to initiation of step (b)). In certain embodiments, the dose of fludarabine is administered to the subject 4 days, 3 days, and/or 2 days prior to step (b) (e.g., prior to initiation of step (b)). In certain embodiments, the dose of fludarabine is administered to the subject 3 days, 2 days, and/or 1 day prior to step (b) (e.g., prior to initiation of step (b)). In certain embodiments, the dose of fludarabine is administered to the subject 5 days prior to step (b) (e.g., prior to initiation of step (b)). In certain embodiments, the dose of fludarabine is administered to the subject 4 days prior to step (b) (e.g., prior to initiation of step (b)). In certain embodiments, the dose of fludarabine is administered to the subject 3 days prior to step (b) (e.g., prior to initiation of step (b)). In certain embodiments, the dose of fludarabine is administered to the subject 5 days, 4 days, and 3 days prior to step (b) (e.g., prior to initiation of step (b)).

In certain embodiments, step (b) is defined as occurring on “Day 0”, and “Day −X” and “Day X” refer to the day occurring X days prior to and after Day 0, respectively. In certain embodiments, the dose of fludarabine is administered to the subject on or prior to Day −1, Day −2, Day −3, Day −4, Day −5, Day −6, Day −7, Day −8, Day −9, or Day −10. In certain embodiments, the dose of fludarabine is administered to the subject on or prior to Day −2. In certain embodiments, the dose of fludarabine is administered to the subject on or prior to Day −3.

In certain embodiments, the dose of fludarabine is administered to the subject on or after Day −10, and before Day 0. In certain embodiments, the dose of fludarabine is administered to the subject on or after Day −7, and before Day 0. In certain embodiments, the dose of fludarabine is administered to the subject on or after Day −5, and before Day 0. In certain embodiments, the dose of fludarabine is administered to the subject on or after Day −5, and before Day −1. In certain embodiments, the dose of fludarabine is administered to the subject on or after Day −5, and before Day −2. In certain embodiments, the preconditioning regimen comprises administering a dose of fludarabine to the subject on Day −10, Day −9, Day −8, Day −7, Day −6, Day −5, Day −4, Day −3, Day −2, and/or Day −1. In certain embodiments, the preconditioning regimen comprises administering a dose of fludarabine to the subject on Day −7, Day −6, Day −5, Day −4, Day −3, Day −2, and/or Day −1. In certain embodiments, the preconditioning regimen comprises administering a dose of fludarabine to the subject on Day −5, Day −4, Day −3, Day −2, and/or Day −1. In certain embodiments, the preconditioning regimen comprises administering a dose of fludarabine to the subject on Day −5, Day −4, and/or Day −3. In certain embodiments, the preconditioning regimen comprises administering a dose of fludarabine to the subject on Day −4, Day −3, and/or Day −2. In certain embodiments, the preconditioning regimen comprises administering a dose of fludarabine to the subject on Day −3, Day −2, and/or Day −1. In certain embodiments, the preconditioning regimen comprises administering a dose of fludarabine to the subject on Day −5. In certain embodiments, the preconditioning regimen comprises administering a dose of fludarabine to the subject on Day −4. In certain embodiments, the preconditioning regimen comprises administering a dose of fludarabine to the subject on Day −3. In certain embodiments, the preconditioning regimen comprises administering a dose of fludarabine to the subject on Day −5, Day −4, and Day −3.

In certain embodiments, a preconditioning regimen of the disclosure comprises administering a dose of cyclophosphamide and a dose of fludarabine to the subject, wherein the total dose of fludarabine administered to the subject is 5-80 mg/m². If, for example, a subject is administered a dose of 12.5 mg/m²/day fludarabine on Day −5, Day −4, and Day −3, then the subject is considered to have been administered a total dose of 37.5 mg/m² fludarabine. If a subject is administered a single dose of 20 mg/m²/day fludarabine on Day −3, then the subject is considered to have been administered a total dose of 20 mg/m² fludarabine. In certain embodiments, the preconditioning regimen comprises administering a total dose of 5-80 mg/m², 5-75 mg/m², 5-67.5 mg/m², 5-60 mg/m², 5-57 mg/m², 5-54 mg/m², 5-51 mg/m², 5-50 mg/m², 5-48 mg/m², 5-45 mg/m², 5-42 mg/m², 5-39 mg/m², 5-37.5 mg/m², 5-36 mg/m², 5-33 mg/m², 5-30 mg/m², 5-27 mg/m², 5-25 mg/m², 5-24 mg/m², 5-21 mg/m², 5-19.5 mg/m², 5-18.75 mg/m², 5-18 mg/m², 5-15 mg/m², 5-12 mg/m², 5-9 mg/m², 5-6 mg/m², 6-80 mg/m², 6-75 mg/m², 6-67.5 mg/m², 6-60 mg/m², 6-57 mg/m², 6-54 mg/m², 6-51 mg/m², 6-50 mg/m², 6-48 mg/m², 6-45 mg/m², 6-42 mg/m², 6-39 mg/m², 6-37.5 mg/m², 6-36 mg/m², 6-33 mg/m², 6-30 mg/m², 6-27 mg/m², 6-25 mg/m², 6-24 mg/m², 6-21 mg/m², 6-19.5 mg/m², 6-18.75 mg/m², 6-18 mg/m², 6-15 mg/m², 6-12 mg/m², 6-9 mg/m², 9-80 mg/m², 9-75 mg/m², 9-67.5 mg/m², 9-60 mg/m², 9-57 mg/m², 9-54 mg/m², 9-51 mg/m², 9-50 mg/m², 9-48 mg/m², 9-45 mg/m², 9-42 mg/m², 9-39 mg/m², 9-37.5 mg/m², 9-36 mg/m², 9-33 mg/m², 9-30 mg/m², 9-27 mg/m², 9-25 mg/m², 9-24 mg/m², 9-21 mg/m², 9-19.5 mg/m², 9-18.75 mg/m², 9-18 mg/m², 9-15 mg/m², 9-12 mg/m², 12-80 mg/m², 12-75 mg/m², 12-67.5 mg/m², 12-60 mg/m², 12-57 mg/m², 12-54 mg/m², 12-51 mg/m², 12-50 mg/m², 12-48 mg/m², 12-45 mg/m², 12-42 mg/m², 12-39 mg/m², 12-37.5 mg/m², 12-36 mg/m², 12-33 mg/m², 12-30 mg/m², 12-27 mg/m², 12-25 mg/m², 12-24 mg/m², 12-21 mg/m², 12-19.5 mg/m², 12-18.75 mg/m², 12-18 mg/m², 12-15 mg/m², 15-80 mg/m², 15-75 mg/m², 15-67.5 mg/m², 15-60 mg/m², 15-57 mg/m², 15-54 mg/m², 15-51 mg/m², 15-50 mg/m², 15-48 mg/m², 15-45 mg/m², 15-42 mg/m², 15-39 mg/m², 15-37.5 mg/m², 15-36 mg/m², 15-33 mg/m², 15-30 mg/m², 15-27 mg/m², 15-25 mg/m², 15-24 mg/m², 15-21 mg/m², 15-19.5 mg/m², 15-18.75 mg/m², 15-18 mg/m², 18-80 mg/m², 18-75 mg/m², 18-67.5 mg/m², 18-60 mg/m², 18-57 mg/m², 18-54 mg/m², 18-51 mg/m², 18-50 mg/m², 18-48 mg/m², 18-45 mg/m², 18-42 mg/m², 18-39 mg/m², 18-37.5 mg/m², 18-36 mg/m², 18-33 mg/m², 18-30 mg/m², 18-27 mg/m², 18-25 mg/m², 18-24 mg/m², 18-21 mg/m², 18-19.5 mg/m², 18-18.75 mg/m², 18.75-80 mg/m², 18.75-75 mg/m², 18.75-67.5 mg/m², 18.75-60 mg/m², 18.75-57 mg/m², 18.75-54 mg/m², 18.75-51 mg/m², 18.75-50 mg/m², 18.75-48 mg/m², 18.75-45 mg/m², 18.75-42 mg/m², 18.75-39 mg/m², 18.75-37.5 mg/m², 18.75-36 mg/m², 18.75-33 mg/m², 18.75-30 mg/m², 18.75-27 mg/m², 18.75-25 mg/m², 18.75-24 mg/m², 18.75-21 mg/m², 18.75-19.5 mg/m², 19.5-80 mg/m², 19.5-75 mg/m², 19.5-67.5 mg/m², 19.5-60 mg/m², 19.5-57 mg/m², 19.5-54 mg/m², 19.5-51 mg/m², 19.5-50 mg/m², 19.5-48 mg/m², 19.5-45 mg/m², 19.5-42 mg/m², 19.5-39 mg/m², 19.5-37.5 mg/m², 19.5-36 mg/m², 19.5-33 mg/m², 19.5-30 mg/m², 19.5-27 mg/m², 19.5-25 mg/m², 19.5-24 mg/m², 19.5-21 mg/m², 21-80 mg/m², 21-75 mg/m², 21-67.5 mg/m², 21-60 mg/m², 21-57 mg/m², 21-54 mg/m², 21-51 mg/m², 21-50 mg/m², 21-48 mg/m², 21-45 mg/m², 21-42 mg/m², 21-39 mg/m², 21-37.5 mg/m², 21-36 mg/m², 21-33 mg/m², 21-30 mg/m², 21-27 mg/m², 21-25 mg/m², 21-24 mg/m², 24-80 mg/m², 24-75 mg/m², 24-67.5 mg/m², 24-60 mg/m², 24-57 mg/m², 24-54 mg/m², 24-51 mg/m², 24-50 mg/m², 24-48 mg/m², 24-45 mg/m², 24-42 mg/m², 24-39 mg/m², 24-37.5 mg/m², 24-36 mg/m², 24-33 mg/m², 24-30 mg/m², 24-27 mg/m², 24-25 mg/m², 25-80 mg/m², 25-75 mg/m², 25-67.5 mg/m², 25-60 mg/m², 25-57 mg/m², 25-54 mg/m², 25-51 mg/m², 25-50 mg/m², 25-48 mg/m², 25-45 mg/m², 25-42 mg/m², 25-39 mg/m², 25-37.5 mg/m², 25-36 mg/m², 25-33 mg/m², 25-30 mg/m², 25-27 mg/m², 27-80 mg/m², 27-75 mg/m², 27-67.5 mg/m², 27-60 mg/m², 27-57 mg/m², 27-54 mg/m², 27-51 mg/m², 27-50 mg/m², 27-48 mg/m², 27-45 mg/m², 27-42 mg/m², 27-39 mg/m², 27-37.5 mg/m², 27-36 mg/m², 27-33 mg/m², 27-30 mg/m², 30-80 mg/m², 30-75 mg/m², 30-67.5 mg/m², 30-60 mg/m², 30-57 mg/m², 30-54 mg/m², 30-51 mg/m², 30-50 mg/m², 30-48 mg/m², 30-45 mg/m², 30-42 mg/m², 30-39 mg/m², 30-37.5 mg/m², 30-36 mg/m², 30-33 mg/m², 33-80 mg/m², 33-75 mg/m², 33-67.5 mg/m², 33-60 mg/m², 33-57 mg/m², 33-54 mg/m², 33-51 mg/m², 33-50 mg/m², 33-48 mg/m², 33-45 mg/m², 33-42 mg/m², 33-39 mg/m², 33-37.5 mg/m², 33-36 mg/m², 36-80 mg/m², 36-75 mg/m², 36-67.5 mg/m², 36-60 mg/m², 36-57 mg/m², 36-54 mg/m², 36-51 mg/m², 36-50 mg/m², 36-48 mg/m², 36-45 mg/m², 36-42 mg/m², 36-39 mg/m², 36-37.5 mg/m², 37.5-80 mg/m², 37.5-75 mg/m², 37.5-67.5 mg/m², 37.5-60 mg/m², 37.5-57 mg/m², 37.5-54 mg/m², 37.5-51 mg/m², 37.5-50 mg/m², 37.5-48 mg/m², 37.5-45 mg/m², 37.5-42 mg/m², 37.5-39 mg/m², 39-80 mg/m², 39-75 mg/m², 39-67.5 mg/m², 39-60 mg/m², 39-57 mg/m², 39-54 mg/m², 39-51 mg/m², 39-50 mg/m², 39-48 mg/m², 39-45 mg/m², 39-42 mg/m², 45-80 mg/m², 45-75 mg/m², 45-67.5 mg/m², 45-60 mg/m², 45-57 mg/m², 45-54 mg/m², 45-51 mg/m², 45-50 mg/m², 45-48 mg/m², 48-80 mg/m², 48-75 mg/m², 48-67.5 mg/m², 48-60 mg/m², 48-57 mg/m², 48-54 mg/m², 48-51 mg/m², 48-50 mg/m², 50-80 mg/m², 50-75 mg/m², 50-67.5 mg/m², 50-60 mg/m², 50-57 mg/m², 50-54 mg/m², 50-51 mg/m², 51-80 mg/m², 51-75 mg/m², 51-67.5 mg/m², 51-60 mg/m², 51-57 mg/m², 51-54 mg/m², 54-80 mg/m², 54-75 mg/m², 54-67.5 mg/m², 54-60 mg/m², 54-57 mg/m², 57-80 mg/m², 57-75 mg/m², 57-67.5 mg/m², 57-60 mg/m², 60-80 mg/m², 60-75 mg/m², 60-67.5 mg/m², 67.5-80 mg/m², 67.5-75 mg/m², or 75-80 mg/m² fludarabine to the subject. In certain embodiments, the preconditioning regimen comprises administering a dose of 5-60 mg/m² fludarabine to the subject. In certain embodiments, the preconditioning regimen comprises administering a dose of 15-50 mg/m² fludarabine to the subject. In certain embodiments, the preconditioning regimen comprises administering a dose of 18.75-45 mg/m² fludarabine to the subject. In certain embodiments, the total dose of fludarabine is administered to the subject in a single day.

In certain embodiments, a preconditioning regimen of the disclosure comprises administering a dose of cyclophosphamide and a dose of fludarabine to the subject, wherein the total dose of fludarabine administered to the subject is less than 90 mg/m². For example, in certain embodiments, the total dose of fludarabine administered to the subject is less than 90 mg/m², less than 80 mg/m², less than 75 mg/m², less than 67.5 mg/m², less than 60 mg/m², less than 57 mg/m², less than 54 mg/m², less than 51 mg/m², less than 50 mg/m², less than 48 mg/m², less than 45 mg/m², less than 42 mg/m², less than 39 mg/m², less than 37.5 mg/m², less than 36 mg/m², less than 33 mg/m², less than 30 mg/m², less than 27 mg/m², less than 25 mg/m², less than 24 mg/m², less than 21 mg/m², less than 19.5 mg/m², less than 18.75 mg/m², less than 18 mg/m², less than 15 mg/m², less than 12 mg/m², less than 9 mg/m², or less than 6 mg/m². In certain embodiments, the total dose of fludarabine administered to the subject is less than 75 mg/m². In certain embodiments, the total dose of fludarabine administered to the subject is less than 60 mg/m². In certain embodiments, the total dose of fludarabine administered to the subject is less than 50 mg/m². In certain embodiments, the total dose of fludarabine administered to the subject is less than 25 mg/m².

In certain embodiments, a preconditioning regimen of the disclosure comprises administering a dose of cyclophosphamide and a dose of fludarabine to the subject, wherein the total dose of fludarabine administered to the subject is 80 mg/m², 75 mg/m², 67.5 mg/m², 60 mg/m², 57 mg/m², 54 mg/m², 51 mg/m², 50 mg/m², 48 mg/m², 45 mg/m², 42 mg/m², 39 mg/m², 37.5 mg/m², 36 mg/m², 33 mg/m², 30 mg/m², 27 mg/m², 25 mg/m², 24 mg/m², 21 mg/m², 19.5 mg/m², 18.75 mg/m², 18 mg/m², 15 mg/m², 12 mg/m², 9 mg/m², or 6 mg/m². In certain embodiments, the total dose of fludarabine administered to the subject is 45 mg/m². In certain embodiments, the total dose of fludarabine administered to the subject is 37.5 mg/m². In certain embodiments, the total dose of fludarabine administered to the subject is 18.75 mg/m².

In certain embodiments, a preconditioning regimen of the disclosure comprises administering a dose of cyclophosphamide and a dose of fludarabine to the subject, wherein the dose of cyclophosphamide and the dose of fludarabine are each independently selected from any of the doses or ranges described herein. The doses of cyclophosphamide and fludarabine can be raised or lowered together or independently. For example, the dose of cyclophosphamide can be increased while the dose of fludarabine is decreased, and the dose of cyclophosphamide can be decreased while the dose of fludarabine is increased. Alternatively, the dose of both cyclophosphamide and fludarabine can be increased or decreased together.

In certain embodiments, a dose of cyclophosphamide and a dose of fludarabine are administered on different days. In certain embodiments, a dose of cyclophosphamide and a dose of fludarabine are administered on the same day, e.g., on Day −5, Day −4, Day −3, Day −2, and/or Day −1. In certain embodiments, a dose of cyclophosphamide and a dose of fludarabine are both administered on Day −4. In certain embodiments, a dose of cyclophosphamide and a dose of fludarabine are both administered on Day −3. In certain embodiments, a dose of cyclophosphamide and a dose of fludarabine are both administered on Day −2. In certain embodiments, a dose of cyclophosphamide and a dose of fludarabine are both administered on Day −1. In certain embodiments, the total dose of cyclophosphamide and the total dose of fludarabine are administered to the subject on the same day. For example, in certain embodiments, the total dose of cyclophosphamide and the total dose of fludarabine are administered on the same day, e.g., on Day −5, Day −4, Day −3, Day −2, or Day −1. In certain embodiments, the total dose of cyclophosphamide and the total dose of fludarabine are both administered on Day −4. In certain embodiments, the total dose of cyclophosphamide and the total dose of fludarabine are both administered on Day −3. In certain embodiments, the total dose of cyclophosphamide and the total dose of fludarabine are both administered on Day −2. In certain embodiments, the total dose of cyclophosphamide and the total dose of fludarabine are both administered on Day −1.

In certain embodiments, a preconditioning regimen of the disclosure comprises administering a dose of cyclophosphamide and a dose of fludarabine to the subject, wherein the dose of cyclophosphamide is 400-600 mg/m²/day and the dose of fludarabine is 10-15 mg/m²/day. For example, in certain embodiments, the dose of cyclophosphamide is 400-600 mg/m²/day (e.g., 400-600 mg/m²/day, 400-550 mg/m²/day, 400-500 mg/m²/day, 400-450 mg/m²/day, 450-600 mg/m²/day, 450-550 mg/m²/day, 450-500 mg/m²/day, 500-600 mg/m²/day, 500-550 mg/m²/day, or 550-600 mg/m²/day) and the dose of fludarabine is 10-15 mg/m²/day (e.g., 10-15 mg/m²/day, 10-14 mg/m²/day, 10-13 mg/m²/day, 10-12.5 mg/m²/day, 10-12 mg/m²/day, 10-11 mg/m²/day, 11-15 mg/m²/day, 11-14 mg/m²/day, 11-13 mg/m²/day, 11-12.5 mg/m²/day, 11-12 mg/m²/day, 12-15 mg/m²/day, 12-14 mg/m²/day, 12-13 mg/m²/day, 12-12.5 mg/m²/day, 12.5-15 mg/m²/day, 12.5-14 mg/m²/day, 12.5-13 mg/m²/day, 13-15 mg/m²/day, 13-14 mg/m²/day, or 14-15 mg/m²/day). In certain embodiments, the dose of cyclophosphamide is 450-550 mg/m²/day and the dose of fludarabine is 11-14 mg/m²/day. In certain embodiments, the dose of cyclophosphamide is 400 mg/m²/day, 450 mg/m²/day, 500 mg/m²/day, 550 mg/m²/day, or 600 mg/m²/day, and the dose of fludarabine is 10 mg/m²/day, 11 mg/m²/day, 12 mg/m²/day, 12.5 mg/m²/day, 13 mg/m²/day, 14 mg/m²/day, or 15 mg/m²/day. In certain embodiments, the dose of cyclophosphamide is 500 mg/m²/day and the dose of fludarabine is 12.5 mg/m²/day. In certain embodiments, the dose of cyclophosphamide is administered on Day −3, and the dose of fludarabine is administered on Days −5, −4, and −3.

In certain embodiments, a preconditioning regimen of the disclosure comprises administering a dose of cyclophosphamide and a dose of fludarabine to the subject, wherein the dose of cyclophosphamide is 400-600 mg/m²/day and the dose of fludarabine is 12-18 mg/m²/day. For example, in certain embodiments, the dose of cyclophosphamide is 400-600 mg/m²/day (e.g., 400-600 mg/m²/day, 400-550 mg/m²/day, 400-500 mg/m²/day, 400-450 mg/m²/day, 450-600 mg/m²/day, 450-550 mg/m²/day, 450-500 mg/m²/day, 500-600 mg/m²/day, 500-550 mg/m²/day, or 550-600 mg/m²/day) and the dose of fludarabine is 12-18 mg/m²/day (e.g., 12-18 mg/m²/day, 12-17 mg/m²/day, 12-16 mg/m²/day, 12-15 mg/m²/day, 12-14 mg/m²/day, 12-13 mg/m²/day, 12-12.5 mg/m²/day, 12.5-18 mg/m²/day, 12.5-17 mg/m²/day, 12.5-16 mg/m²/day, 12.5-15 mg/m²/day, 12.5-14 mg/m²/day, 12.5-13 mg/m²/day, 13-18 mg/m²/day, 13-17 mg/m²/day, 13-16 mg/m²/day, 13-15 mg/m²/day, 13-14 mg/m²/day, 14-18 mg/m²/day, 14-17 mg/m²/day, 14-16 mg/m²/day, 14-15 mg/m²/day, 15-18 mg/m²/day, 15-17 mg/m²/day, 15-16 mg/m²/day, 16-18 mg/m²/day, 16-17 mg/m²/day, or 17-18 mg/m²/day). In certain embodiments, the dose of cyclophosphamide is 450-550 mg/m²/day and the dose of fludarabine is 14-16 mg/m²/day. In certain embodiments, the dose of cyclophosphamide is 400 mg/m²/day, 450 mg/m²/day, 500 mg/m²/day, 550 mg/m²/day, or 600 mg/m²/day, and the dose of fludarabine is 12 mg/m²/day, 12.5 mg/m²/day, 13 mg/m²/day, 14 mg/m²/day, 15 mg/m²/day, 16 mg/m²/day, 17 mg/m²/day, or 18 mg/m²/day. In certain embodiments, the dose of cyclophosphamide is 500 mg/m²/day and the dose of fludarabine is 15 mg/m²/day. In certain embodiments, the dose of cyclophosphamide is administered on Day −3, and the dose of fludarabine is administered on Days −5, −4, and −3.

In certain embodiments, a preconditioning regimen of the disclosure comprises administering a dose of cyclophosphamide and a dose of fludarabine to the subject, wherein the dose of cyclophosphamide is 800-1,200 mg/m²/day, and the dose of fludarabine is 10-15 mg/m²/day. For example, in certain embodiments, the dose of cyclophosphamide is 800-1,200 mg/m²/day (e.g., 800-1,200 mg/m²/day, 800-1,100 mg/m²/day, 800-1,000 mg/m²/day, 800-950 mg/m²/day, 800-900 mg/m²/day, 800-850 mg/m²/day, 850-1,200 mg/m²/day, 850-1,100 mg/m²/day, 850-1,000 mg/m²/day, 850-950 mg/m²/day, 850-900 mg/m²/day, 900-1,200 mg/m²/day, 900-1,100 mg/m²/day, 900-1,000 mg/m²/day, 900-950 mg/m²/day, 950-1,200 mg/m²/day, 950-1,100 mg/m²/day, 950-1,000 mg/m²/day, 1,000-1,200 mg/m²/day, 1,000-1,100 mg/m²/day, or 1,100-1,200 mg/m²/day) and the dose of fludarabine is 10-15 mg/m²/day (e.g., 10-15 mg/m²/day, 10-14 mg/m²/day, 10-13 mg/m²/day, 10-12.5 mg/m²/day, 10-12 mg/m²/day, 10-11 mg/m²/day, 11-15 mg/m²/day, 11-14 mg/m²/day, 11-13 mg/m²/day, 11-12.5 mg/m²/day, 11-12 mg/m²/day, 12-15 mg/m²/day, 12-14 mg/m²/day, 12-13 mg/m²/day, 12-12.5 mg/m²/day, 12.5-15 mg/m²/day, 12.5-14 mg/m²/day, 12.5-13 mg/m²/day, 13-15 mg/m²/day, 13-14 mg/m²/day, or 14-15 mg/m²/day). In certain embodiments, the dose of cyclophosphamide is 900-1,100 mg/m²/day and the dose of fludarabine is 11-14 mg/m²/day. In certain embodiments, the dose of cyclophosphamide is 950-1,050 mg/m²/day and the dose of fludarabine is 12-13 mg/m²/day. In certain embodiments, the dose of cyclophosphamide is 800 mg/m²/day, 850 mg/m²/day, 900 mg/m²/day, 950 mg/m²/day, 1,000 mg/m²/day, 1,100 mg/m²/day, or 1,200 mg/m²/day, and the dose of fludarabine is 10 mg/m²/day, 11 mg/m²/day, 12 mg/m²/day, 12.5 mg/m²/day, 13 mg/m²/day, 14 mg/m²/day, or 15 mg/m²/day. In certain embodiments, the dose of cyclophosphamide is 1,000 mg/m²/day and the dose of fludarabine is 12.5 mg/m²/day. In certain embodiments, the dose of cyclophosphamide is administered on Day −3, and the dose of fludarabine is administered on Days −5, −4, and −3.

In certain embodiments, a preconditioning regimen of the disclosure comprises administering a dose of cyclophosphamide and a dose of fludarabine to the subject, wherein the dose of cyclophosphamide is 150-350 mg/m²/day, and the dose of fludarabine is 5-7.5 mg/m²/day. For example, in certain embodiments, the dose of cyclophosphamide is 150-350 mg/m²/day (e.g., 150-350 mg/m²/day, 150-300 mg/m²/day, 150-250 mg/m²/day, 150-200 mg/m²/day, 200-350 mg/m²/day, 200-300 mg/m²/day, 200-250 mg/m²/day, 250-350 mg/m²/day, 250-300 mg/m²/day, or 300-350 mg/m²/day) and the dose of fludarabine is 5-7.5 mg/m²/day (e.g., 5-7.5 mg/m²/day, 5-7 mg/m²/day, 5-6.5 mg/m²/day, 5-6.25 mg/m²/day, 5-6 mg/m²/day, 6-7.5 mg/m²/day, 6-7 mg/m²/day, 6-6.5 mg/m²/day, 6-6.25 mg/m²/day, 6.25-7.5 mg/m²/day, 6.25-7 mg/m²/day, 6.25-6.5 mg/m²/day, 6.5-7.5 mg/m²/day, 6.5-7 mg/m²/day, or 7-7.5 mg/m²/day). In certain embodiments, the dose of cyclophosphamide is 200-300 mg/m²/day and the dose of fludarabine is 6-7 mg/m²/day. In certain embodiments, the dose of cyclophosphamide is 150 mg/m²/day, 175 mg/m²/day, 200 mg/m²/day, 225 mg/m²/day, 250 mg/m²/day, 275 mg/m²/day, 300 mg/m²/day, or 350 mg/m²/day, and the dose of fludarabine is 5 mg/m²/day, 6 mg/m²/day, 6.25 mg/m²/day, 6.5 mg/m²/day, 7 mg/m²/day, or 7.5 mg/m²/day. In certain embodiments, the dose of cyclophosphamide is 250 mg/m²/day and the dose of fludarabine is 6.25 mg/m²/day. In certain embodiments, the dose of cyclophosphamide is administered on Day −3, and the dose of fludarabine is administered on Days −5, −4, and −3.

In certain embodiments, a preconditioning regimen of the disclosure comprises administering a dose of cyclophosphamide and a dose of fludarabine to the subject, wherein the dose of cyclophosphamide is 400-600 mg/m²/day, and the dose of fludarabine is 5-7.5 mg/m²/day. For example, in certain embodiments, the dose of cyclophosphamide is 400-600 mg/m²/day (e.g., 400-600 mg/m²/day, 400-550 mg/m²/day, 400-500 mg/m²/day, 400-450 mg/m²/day, 450-600 mg/m²/day, 450-550 mg/m²/day, 450-500 mg/m²/day, 500-600 mg/m²/day, 500-550 mg/m²/day, or 550-600 mg/m²/day) and the dose of fludarabine is 5-7.5 mg/m²/day (e.g., 5-7.5 mg/m²/day, 5-7 mg/m²/day, 5-6.5 mg/m²/day, 5-6.25 mg/m²/day, 5-6 mg/m²/day, 6-7.5 mg/m²/day, 6-7 mg/m²/day, 6-6.5 mg/m²/day, 6-6.25 mg/m²/day, 6.25-7.5 mg/m²/day, 6.25-7 mg/m²/day, 6.25-6.5 mg/m²/day, 6.5-7.5 mg/m²/day, 6.5-7 mg/m²/day, or 7-7.5 mg/m²/day). In certain embodiments, the dose of cyclophosphamide is 450-550 mg/m²/day and the dose of fludarabine is 6-7 mg/m²/day. In certain embodiments, the dose of cyclophosphamide is 400 mg/m²/day, 450 mg/m²/day, 500 mg/m²/day, 550 mg/m²/day, or 600 mg/m²/day, and the dose of fludarabine is 5 mg/m²/day, 6 mg/m²/day, 6.25 mg/m²/day, 6.5 mg/m²/day, 7 mg/m²/day, or 7.5 mg/m²/day. In certain embodiments, the dose of cyclophosphamide is 500 mg/m²/day and the dose of fludarabine is 6.25 mg/m²/day. In certain embodiments, the dose of cyclophosphamide is administered on Day −3, and the dose of fludarabine is administered on Days −5, −4, and −3. In certain embodiments, the dose of fludarabine is administered on Days −5, −4, and −3, and the dose of cyclophosphamide is administered on Days −4 and −3.

In certain embodiments, a preconditioning regimen of the disclosure comprises administering a dose of cyclophosphamide and a dose of fludarabine to the subject, wherein the dose of cyclophosphamide is 800-1,200 mg/m²/day and the dose of fludarabine is 5-7.5 mg/m²/day. For example, in certain embodiments, the dose of cyclophosphamide is 800-1,200 mg/m²/day (e.g., 800-1,200 mg/m²/day, 800-1,100 mg/m²/day, 800-1,000 mg/m²/day, 800-950 mg/m²/day, 800-900 mg/m²/day, 800-850 mg/m²/day, 850-1,200 mg/m²/day, 850-1,100 mg/m²/day, 850-1,000 mg/m²/day, 850-950 mg/m²/day, 850-900 mg/m²/day, 900-1,200 mg/m²/day, 900-1,100 mg/m²/day, 900-1,000 mg/m²/day, 900-950 mg/m²/day, 950-1,200 mg/m²/day, 950-1,100 mg/m²/day, 950-1,000 mg/m²/day, 1,000-1,200 mg/m²/day, 1,000-1,100 mg/m²/day, or 1,100-1,200 mg/m²/day) and the dose of fludarabine is 5-7.5 mg/m²/day (e.g., 5-7.5 mg/m²/day, 5-7 mg/m²/day, 5-6.5 mg/m²/day, 5-6.25 mg/m²/day, 5-6 mg/m²/day, 6-7.5 mg/m²/day, 6-7 mg/m²/day, 6-6.5 mg/m²/day, 6-6.25 mg/m²/day, 6.25-7.5 mg/m²/day, 6.25-7 mg/m²/day, 6.25-6.5 mg/m²/day, 6.5-7.5 mg/m²/day, 6.5-7 mg/m²/day, or 7-7.5 mg/m²/day). In certain embodiments, the dose of cyclophosphamide is 900-1,100 mg/m²/day and the dose of fludarabine is 6-7 mg/m²/day. In certain embodiments, the dose of cyclophosphamide is 950-1,050 mg/m²/day and the dose of fludarabine is 6-7 mg/m²/day. In certain embodiments, the dose of cyclophosphamide is 800 mg/m²/day, 850 mg/m²/day, 900 mg/m²/day, 950 mg/m²/day, 1,000 mg/m²/day, 1,100 mg/m²/day, or 1,200 mg/m²/day, and the dose of fludarabine is 5 mg/m²/day, 6 mg/m²/day, 6.25 mg/m²/day, 6.5 mg/m²/day, 7 mg/m²/day, or 7.5 mg/m²/day. In certain embodiments, the dose of cyclophosphamide is 1,000 mg/m²/day and the dose of fludarabine is 6.25 mg/m²/day. In certain embodiments, the dose of cyclophosphamide is administered on Day −3, and the dose of fludarabine is administered on Days −5, −4, and −3.

In certain embodiments, a preconditioning regimen of the disclosure comprises administering a dose of cyclophosphamide and a dose of fludarabine to the subject, wherein the total dose of cyclophosphamide is 400-600 mg/m² and the total dose of fludarabine is 30-45 mg/m². For example, in certain embodiments, the total dose of cyclophosphamide is 400-600 mg/m² (e.g., 400-600 mg/m², 400-550 mg/m², 400-500 mg/m², 400-450 mg/m², 450-600 mg/m², 450-550 mg/m², 450-500 mg/m², 500-600 mg/m², 500-550 mg/m², or 550-600 mg/m²) and the total dose of fludarabine is 25-50 mg/m²/day (e.g., 30-45 mg/m², 30-42 mg/m², 30-39 mg/m², 30-37.5 mg/m², 30-36 mg/m², 30-33 mg/m², 33-45 mg/m², 33-42 mg/m², 33-39 mg/m², 33-37.5 mg/m², 33-36 mg/m², 36-45 mg/m², 36-42 mg/m², 36-39 mg/m², 36-37.5 mg/m², 37.5-45 mg/m², 37.5-42 mg/m², 37.5-39 mg/m², 39-45 mg/m², 39-42 mg/m², or 42-45 mg/M²). In certain embodiments, the total dose of cyclophosphamide is 450-550 mg/m² and the total dose of fludarabine is 35-40 mg/m². In certain embodiments, the total dose of cyclophosphamide is 400 mg/m², 450 mg/m², 500 mg/m², 550 mg/m², or 600 mg/m², and the total dose of fludarabine is 45 mg/m², 42 mg/m², 39 mg/m², 37.5 mg/m², 36 mg/m², 33 mg/m², or 30 mg/m². In certain embodiments, the total dose of cyclophosphamide is 500 mg/m² and the total dose of fludarabine is 37.5 mg/m².

In certain embodiments, a preconditioning regimen of the disclosure comprises administering a dose of cyclophosphamide and a dose of fludarabine to the subject, wherein the total dose of cyclophosphamide is 400-600 mg/m² and the total dose of fludarabine is 36-54 mg/m². For example, in certain embodiments, the total dose of cyclophosphamide is 400-600 mg/m² (e.g., 400-600 mg/m², 400-550 mg/m², 400-500 mg/m², 400-450 mg/m², 450-600 mg/m², 450-550 mg/m², 450-500 mg/m², 500-600 mg/m², 500-550 mg/m², or 550-600 mg/m²) and the total dose of fludarabine is 36-54 mg/m²/day (e.g., 36-54 mg/m², 36-51 mg/m², 36-50 mg/m², 36-48 mg/m², 36-45 mg/m², 36-42 mg/m², 36-39 mg/m², 36-37.5 mg/m², 37.5-54 mg/m², 37.5-51 mg/m², 37.5-50 mg/m², 37.5-48 mg/m², 37.5-45 mg/m², 37.5-42 mg/m², 37.5-39 mg/m², 39-54 mg/m², 39-51 mg/m², 39-50 mg/m², 39-48 mg/m², 39-45 mg/m², 39-42 mg/m², 45-54 mg/m², 45-51 mg/m², 45-50 mg/m², 45-48 mg/m², 48-54 mg/m², 48-51 mg/m², 48-50 mg/m², 50-54 mg/m², 50-51 mg/m², or 51-54 mg/M²). In certain embodiments, the total dose of cyclophosphamide is 450-550 mg/m² and the total dose of fludarabine is 40-50 mg/m². In certain embodiments, the total dose of cyclophosphamide is 400 mg/m², 450 mg/m², 500 mg/m², 550 mg/m², or 600 mg/m², and the total dose of fludarabine is 54 mg/m², 51 mg/m², 50 mg/m², 48 mg/m², 45 mg/m², 42 mg/m², 39 mg/m², 37.5 mg/m², or 36 mg/m². In certain embodiments, the total dose of cyclophosphamide is 500 mg/m² and the total dose of fludarabine is 45 mg/m².

In certain embodiments, a preconditioning regimen of the disclosure comprises administering a dose of cyclophosphamide and a dose of fludarabine to the subject, wherein the total dose of cyclophosphamide is 800-1,200 mg/m² and the total dose of fludarabine is 30-45 mg/m². For example, in certain embodiments, the total dose of cyclophosphamide is 800-1,200 mg/m² (e.g., 800-1,200 mg/m², 800-1,100 mg/m², 800-1,000 mg/m², 800-950 mg/m², 800-900 mg/m², 800-850 mg/m², 850-1,200 mg/m², 850-1,100 mg/m², 850-1,000 mg/m², 850-950 mg/m², 850-900 mg/m², 900-1,200 mg/m², 900-1,100 mg/m², 900-1,000 mg/m², 900-950 mg/m², 950-1,200 mg/m², 950-1,100 mg/m², 950-1,000 mg/m², 1,000-1,200 mg/m², 1,000-1,100 mg/m², or 1,100-1,200 mg/m²) and the total dose of fludarabine is 25-50 mg/m²/day (e.g., 30-45 mg/m², 30-42 mg/m², 30-39 mg/m², 30-37.5 mg/m², 30-36 mg/m², 30-33 mg/m², 33-45 mg/m², 33-42 mg/m², 33-39 mg/m², 33-37.5 mg/m², 33-36 mg/m², 36-45 mg/m², 36-42 mg/m², 36-39 mg/m², 36-37.5 mg/m², 37.5-45 mg/m², 37.5-42 mg/m², 37.5-39 mg/m², 39-45 mg/m², 39-42 mg/m², or 42-45 mg/M²). In certain embodiments, the total dose of cyclophosphamide is 900-1,100 mg/m² and the total dose of fludarabine is 35-40 mg/m². In certain embodiments, the total dose of cyclophosphamide is 950-1,050 mg/m² and the total dose of fludarabine is 35-40 mg/m². In certain embodiments, the total dose of cyclophosphamide is 800 mg/m², 850 mg/m², 900 mg/m², 950 mg/m², 1,000 mg/m², 1,100 mg/m², or 1,200 mg/m², and the total dose of fludarabine is 45 mg/m², 42 mg/m², 39 mg/m², 37.5 mg/m², 36 mg/m², 33 mg/m², or 30 mg/m². In certain embodiments, the total dose of cyclophosphamide is 1,000 mg/m² and the total dose of fludarabine is 37.5 mg/m².

In certain embodiments, a preconditioning regimen of the disclosure comprises administering a dose of cyclophosphamide and a dose of fludarabine to the subject, wherein the total dose of cyclophosphamide is 150-350 mg/m², and the total dose of fludarabine is 12-24 mg/m². For example, in certain embodiments, the total dose of cyclophosphamide is 150-350 mg/m² (e.g., 150-350 mg/m², 150-300 mg/m², 150-250 mg/m², 150-200 mg/m², 200-350 mg/m², 200-300 mg/m², 200-250 mg/m², 250-350 mg/m², 250-300 mg/m², or 300-350 mg/m²) and the total dose of fludarabine is 12-24 mg/m²/day (e.g., 12-24 mg/m², 12-21 mg/m², 12-19.5 mg/m², 12-18.75 mg/m², 12-18 mg/m², 12-15 mg/m², 15-24 mg/m², 15-21 mg/m², 15-19.5 mg/m², 15-18.75 mg/m², 15-18 mg/m², 18-24 mg/m², 18-21 mg/m², 18-19.5 mg/m², 18-18.75 mg/m², 18.75-24 mg/m², 18.75-21 mg/m², 18.75-19.5 mg/m², 19.5-24 mg/m², 19.5-21 mg/m², or 21-24 mg/M²). In certain embodiments, the total dose of cyclophosphamide is 200-300 mg/m² and the total dose of fludarabine is 15-21 mg/m². In certain embodiments, the total dose of cyclophosphamide is 200-300 mg/m² and the total dose of fludarabine is 18-19.5 mg/m². In certain embodiments, the total dose of cyclophosphamide is 150 mg/m², 175 mg/m², 200 mg/m², 225 mg/m², 250 mg/m², 275 mg/m², 300 mg/m², or 350 mg/m², and the total dose of fludarabine is 24 mg/m², 21 mg/m², 19.5 mg/m², 18.75 mg/m², 18 mg/m², 15 mg/m², or 12 mg/m². In certain embodiments, the total dose of cyclophosphamide is 250 mg/m² and the total dose of fludarabine is 18.75 mg/m².

In certain embodiments, a preconditioning regimen of the disclosure comprises administering a dose of cyclophosphamide and a dose of fludarabine to the subject, wherein the total dose of cyclophosphamide is 400-600 mg/m², and the total dose of fludarabine is 12-24 mg/m². For example, in certain embodiments, the total dose of cyclophosphamide is 400-600 mg/m² (e.g., 400-600 mg/m², 400-550 mg/m², 400-500 mg/m², 400-450 mg/m², 450-600 mg/m², 450-550 mg/m², 450-500 mg/m², 500-600 mg/m², 500-550 mg/m², or 550-600 mg/m²) and the total dose of fludarabine is 12-24 mg/m²/day (e.g., 12-24 mg/m², 12-21 mg/m², 12-19.5 mg/m², 12-18.75 mg/m², 12-18 mg/m², 12-15 mg/m², 15-24 mg/m², 15-21 mg/m², 15-19.5 mg/m², 15-18.75 mg/m², 15-18 mg/m², 18-24 mg/m², 18-21 mg/m², 18-19.5 mg/m², 18-18.75 mg/m², 18.75-24 mg/m², 18.75-21 mg/m², 18.75-19.5 mg/m², 19.5-24 mg/m², 19.5-21 mg/m², or 21-24 mg/M²). In certain embodiments, the total dose of cyclophosphamide is 450-550 mg/m² and the total dose of fludarabine is 15-21 mg/m². In certain embodiments, the total dose of cyclophosphamide is 450-550 mg/m² and the total dose of fludarabine is 18-19.5 mg/m². In certain embodiments, the total dose of cyclophosphamide is 400 mg/m², 450 mg/m², 500 mg/m², 550 mg/m², or 600 mg/m², and the total dose of fludarabine is 24 mg/m², 21 mg/m², 19.5 mg/m², 18.75 mg/m², 18 mg/m², 15 mg/m², or 12 mg/m². In certain embodiments, the total dose of cyclophosphamide is 500 mg/m² and the total dose of fludarabine is 18.75 mg/m².

In certain embodiments, a preconditioning regimen of the disclosure comprises administering a dose of cyclophosphamide and a dose of fludarabine to the subject, wherein the total dose of cyclophosphamide is 800-1,200 mg/m², and the total dose of fludarabine is 12-24 mg/m². For example, in certain embodiments, the total dose of cyclophosphamide is 800-1,200 mg/m² (e.g., 800-1,200 mg/m², 800-1,100 mg/m², 800-1,000 mg/m², 800-950 mg/m², 800-900 mg/m², 800-850 mg/m², 850-1,200 mg/m², 850-1,100 mg/m², 850-1,000 mg/m², 850-950 mg/m², 850-900 mg/m², 900-1,200 mg/m², 900-1,100 mg/m², 900-1,000 mg/m², 900-950 mg/m², 950-1,200 mg/m², 950-1,100 mg/m², 950-1,000 mg/m², 1,000-1,200 mg/m², 1,000-1,100 mg/m², or 1,100-1,200 mg/m²) and the total dose of fludarabine is 12-24 mg/m²/day (e.g., 12-24 mg/m², 12-21 mg/m², 12-19.5 mg/m², 12-18.75 mg/m², 12-18 mg/m², 12-15 mg/m², 15-24 mg/m², 15-21 mg/m², 15-19.5 mg/m², 15-18.75 mg/m², 15-18 mg/m², 18-24 mg/m², 18-21 mg/m², 18-19.5 mg/m², 18-18.75 mg/m², 18.75-24 mg/m², 18.75-21 mg/m², 18.75-19.5 mg/m², 19.5-24 mg/m², 19.5-21 mg/m², or 21-24 mg/M²). In certain embodiments, the total dose of cyclophosphamide is 900-1,100 mg/m² and the total dose of fludarabine is 15-21 mg/m². In certain embodiments, the total dose of cyclophosphamide is 950-1,050 mg/m² and the total dose of fludarabine is 18-19.5 mg/m². In certain embodiments, the total dose of cyclophosphamide is 800 mg/m², 850 mg/m², 900 mg/m², 950 mg/m², 1,000 mg/m², 1,100 mg/m², or 1,200 mg/m², and the total dose of fludarabine is 24 mg/m², 21 mg/m², 19.5 mg/m², 18.75 mg/m², 18 mg/m², 15 mg/m², or 12 mg/m². In certain embodiments, the total dose of cyclophosphamide is 1,000 mg/m² and the total dose of fludarabine is 18.75 mg/m².

Certain exemplary preconditioning regimens of the disclosure are set forth in TABLE 1. “FLU” and “CY” represent fludarabine and cyclophosphamide, respectively. With the exception of the right-most column, FLU and CY doses in TABLE 1 are indicated as mg/m²/day. Total doses (rightmost column of TABLE 1) are indicated as mg/m².

TABLE 1
Exemplary cyclophosphamide/fludarabine preconditioning regimens

| | | Day −7 | | | | Day −2 | | |
|---|---|---|---|---|---|---|---|---|
| | | to | | | | to | | |
| Regimen | | Day −6 | Day −5 | Day −4 | Day −3 | Day −1 | Total | |
| 1 | FLU | | 12.5 | 12.5 | 12.5 | | 37.5 | mg/m² |
| | CY | | | | 500 | | 500 | mg/m² |
| 2 | FLU | | 15 | 15 | 15 | | 45 | mg/m² |
| | CY | | | | 500 | | 500 | mg/m² |
| 3 | FLU | | 12.5 | 12.5 | 12.5 | | 37.5 | mg/m² |
| | CY | | | | 1,000 | | 1,000 | mg/m² |
| 4 | FLU | | 6.25 | 6.25 | 6.25 | | 18.75 | mg/m² |
| | CY | | | | 250 | | 250 | mg/m² |
| 5 | FLU | | 6.25 | 6.25 | 6.25 | | 18.75 | mg/m² |
| | CY | | | | 500 | | 500 | mg/m² |
| 6 | FLU | | 6.25 | 6.25 | 6.25 | | 18.75 | mg/m² |
| | CY | | | | 1,000 | | 1,000 | mg/m² |
| 7 | FLU | | 6.25 | 6.25 | 6.25 | | 18.75 | mg/m² |
| | CY | | | 500 | 500 | | 1,000 | mg/m² |

The disclosure further relates to pharmaceutical compositions comprising a dose of cyclophosphamide and/or a dose of fludarabine as disclosed herein. In certain embodiments, a dose of cyclophosphamide and a dose of fludarabine as disclosed herein are present in the same pharmaceutical composition. In certain embodiments, a dose of cyclophosphamide and a dose of fludarabine are present in separate pharmaceutical compositions. In certain embodiments, the pharmaceutical compositions disclosed herein further comprise a pharmaceutically acceptable carrier.

In certain embodiments, a dose of cyclophosphamide and a dose of fludarabine are administered to a subject separately. In certain embodiments, a dose of cyclophosphamide and a dose of fludarabine are administered to a subject simultaneously. In certain embodiments, a dose of cyclophosphamide and a dose of fludarabine are administered to a subject sequentially. In certain embodiments, a dose of cyclophosphamide is administered intravenously. In certain embodiments, a dose of fludarabine is administered intravenously.

b. Cell Therapy

In certain embodiments, a therapeutic method described herein comprises administering an immune cell therapy to a subject. For example, in certain embodiments, a therapeutic method of treatment described herein comprises administering to the subject an immune cell composition comprising immune cells (e.g., T cells, B cells, NK cells, tumor-infiltrating lymphocytes, and/or dendritic cells). In certain embodiments, the immune cell composition comprises NK cells. In certain embodiments, the immune cell composition comprises T cells, e.g., CD8+ T cells. In certain embodiments, at least 40%, at least 50%, at least 60%, at least 70%, at least 80%, at least 90%, at least 95%, or at least 99% of the cells in the immune cell composition are CD8+ T cells. In certain embodiments, the immune cell composition further comprises CD4+ T cells, optionally wherein at least 20%, at least 30%, at least 40%, or at least 50% of the cells in the immune cell composition are CD4+ T cells.

Immune cells for use in immune cell therapy can be obtained from a blood sample of a patient by apheresis. In certain embodiments, a lymphocyte-rich fraction and a monocyte-rich fraction can be acquired by elutriating peripheral blood mononuclear cells (PBMCs) of the patient. The monocyte-rich fraction can then be used to prepare antigen-presenting cells (APCs) for priming T cells, which can be obtained from the lymphocyte-rich fraction. Exemplary methods are described in WO2020055931 and U.S. Pat. Nos. 9,963,677 and 9,642,906. In certain embodiments, the immune cell therapy is autologous, i.e., immune cells obtained from a patient, after in vitro culture, are administered to the same patient. In certain embodiments, the immune cell therapy is allogeneic, optionally wherein the immune cells are genetically engineered to abrogate expression via component inactivation of class I MHC (e.g., β2M), class II (e.g., RFXANK), or TCR (e.g. TRAC, or CD3).

In certain embodiments, the immune cell for use in cell therapy is a T cell, e.g., a cytotoxic T cell, a helper T cell, a memory T cell, a regulatory T cell, a gamma delta T cell, a natural killer cell, a cytokine induced killer cell, a cell line thereof, a T memory stem cell, or another T effector cell. In certain embodiments, the T cell specifically targets a cell expressing the targeted antigen, e.g., a B cell expressing CD19. In certain embodiments, the T cell is autologous to the subject. In other embodiments, the T cell is allogeneic to the subject.

Immune cells (e.g., T cells, e.g., CD8+ T cells) can have antigen specificity via a receptor expressed on the cell surface. T cells can naturally express T cell receptors (TCRs), such as αβTCRs or γδTCRs, and can be “trained” to express TCRs that target a given antigen or epitope. Immune cells can also be genetically engineered to express a recombinant TCR or a chimeric antigen receptor (CAR). Where the therapeutic methods disclosed herein are used to treat an autoimmune disease, the TCR or CAR can target one or more antigens or epitopes thereof present on an immune cell surface. In certain embodiments, the immune cells are genetically engineered to inactivate their endogenous TCRs, for example, by knocking out the TRAC or TRBC gene, to reduce ligand-independent tonic T cell signaling and enhances T cell potency.

In certain embodiments, the immune cells for use in immune cell therapy (e.g., T cells or NK cells) express a CAR as described herein. In certain embodiments, the immune cells for use in immune cell therapy express a recombinant TCR. Generally, an αβTCR is capable of binding a peptide presented by a major histocompatibility complex (MHC) molecule, whereas a γδTCR does not require MHC-mediated antigen presentation. In certain embodiments, the TCR comprises variable α and β chains (also known as TCRα and TCRβ, respectively) or variable γ and δ chains (also known as TCRγ and TCRδ, respectively), or a portion thereof that binds the antigen (e.g., peptide-MHC complex) comprising the α and β chain variable domains or the γ and δ chain variable domains. Generally, the variable domains of a TCR comprise complementarity determining regions involved in recognition of the antigen (e.g., peptide-MHC complex). In certain embodiments, the TCR expressed by the immune cells is cloned from a naturally occurring T cell.

The immune cells for use in immune cell therapy can be genetically engineered to express a CAR or TCR by introducing a nucleic acid encoding the CAR or TCR. In certain embodiments, the nucleic acid is a DNA molecule (e.g., a cDNA molecule). In certain embodiments, the nucleic acid further comprises an expression control sequence (e.g., promoter and/or enhancer) operably linked to the CAR or TCR coding sequence. In certain embodiments, the immune cells are transduced by a vector, such as a viral vector (e.g., AAV vector, lentiviral vector, or adenoviral vector) or a non-viral vector (e.g., plasmid), comprising a nucleic acid encoding the CAR or TCR. In certain embodiments, the nucleic acid is an RNA molecule (e.g., an mRNA molecule). Methods for generating and modifying mRNA for use in transfection are disclosed in U.S. Pat. Nos. 8,278,036, 8,883,506, and 8,716,465. In certain embodiments, the nucleic acid encodes an amino acid sequence comprising a signal peptide at the N-terminus of the CAR or TCR. Such signal peptide can facilitate cell surface localization of the CAR or TCR when it is expressed in an effector cell, and is cleaved from the CAR during cellular processing.

In certain embodiments, the therapeutic methods of the disclosure comprise administering an effective amount of immune cells (e.g., CAR immune cells, e.g., CAR T cells) to a subject in need thereof. In certain embodiments, the effective amount of immune cells is from 1×10⁵ cells/kg bodyweight to 1×10⁸ cells/kg bodyweight. For example, in certain embodiments, the effective amount of immune cells is from 1×10⁵ to 1×10⁸ cells/kg, from 1×10⁵ to 9×10⁷ cells/kg, from 1×10⁵ to 6×10⁷ cells/kg, from 1×10⁵ to 3×10⁷ cells/kg, from 1×10⁵ to 1×10⁷ cells/kg, from 1×10⁵ to 9×10⁶ cells/kg, from 1×10⁵ to 8×10⁶ cells/kg, from 1×10⁵ to 7×10⁶ cells/kg, from 1×10⁵ to 6×10⁶ cells/kg, from 1×10⁵ to 5×10⁶ cells/kg, from 1×10⁵ to 4×10⁶ cells/kg, from 1×10⁵ to 3×10⁶ cells/kg, from 1×10⁵ to 2×10⁶ cells/kg, from 1×10⁵ to 1×10⁶ cells/kg, from 1×10⁵ to 9×10⁵ cells/kg, from 1×10⁵ to 6×10⁵ cells/kg, from 1×10⁵ to 3×10⁵ cells/kg, from 3×10⁵ to 1×10⁸ cells/kg, from 3×10⁵ to 9×10⁷ cells/kg, from 3×10⁵ to 6×10⁷ cells/kg, from 3×10⁵ to 3×10⁷ cells/kg, from 3×10⁵ to 1×10⁷ cells/kg, from 3×10⁵ to 9×10⁶ cells/kg, from 3×10⁵ to 8×10⁶ cells/kg, from 3×10⁵ to 7×10⁶ cells/kg, from 3×10⁵ to 6×10⁶ cells/kg, from 3×10⁵ to 5×10⁶ cells/kg, from 3×10⁵ to 4×10⁶ cells/kg, from 3×10⁵ to 3×10⁶ cells/kg, from 3×10⁵ to 2×10⁶ cells/kg, from 3×10⁵ to 1×10⁶ cells/kg, from 3×10⁵ to 9×10⁵ cells/kg, from 3×10⁵ to 6×10⁵ cells/kg, from 6×10⁵ to 1×10⁸ cells/kg, from 6×10⁵ to 9×10⁷ cells/kg, from 6×10⁵ to 6×10⁷ cells/kg, from 6×10⁵ to 3×10⁷ cells/kg, from 6×10⁵ to 1×10⁷ cells/kg, from 6×10⁵ to 9×10⁶ cells/kg, from 6×10⁵ to 8×10⁶ cells/kg, from 6×10⁵ to 7×10⁶ cells/kg, from 6×10⁵ to 6×10⁶ cells/kg, from 6×10⁵ to 5×10⁶ cells/kg, from 6×10⁵ to 4×10⁶ cells/kg, from 6×10⁵ to 3×10⁶ cells/kg, from 6×10⁵ to 2×10⁶ cells/kg, from 6×10⁵ to 1×10⁶ cells/kg, from 6×10⁵ to 9×10⁵ cells/kg, from 9×10⁵ to 1×10⁸ cells/kg, from 9×10⁵ to 9×10⁷ cells/kg, from 9×10⁵ to 6×10⁷ cells/kg, from 9×10⁵ to 3×10⁷ cells/kg, from 9×10⁵ to 1×10⁷ cells/kg, from 9×10⁵ to 9×10⁶ cells/kg, from 9×10⁵ to 8×10⁶ cells/kg, from 9×10⁵ to 7×10⁶ cells/kg, from 9×10⁵ to 6×10⁶ cells/kg, from 9×10⁵ to 5×10⁶ cells/kg, from 9×10⁵ to 4×10⁶ cells/kg, from 9×10⁵ to 3×10⁶ cells/kg, from 9×10⁵ to 2×10⁶ cells/kg, from 9×10⁵ to 1×10⁶ cells/kg, from 1×10⁶ to 1×10⁸ cells/kg, from 1×10⁶ to 9×10⁷ cells/kg, from 1×10⁶ to 6×10⁷ cells/kg, from 1×10⁶ to 3×10⁷ cells/kg, from 1×10⁶ to 1×10⁷ cells/kg, from 1×10⁶ to 9×10⁶ cells/kg, from 1×10⁶ to 8×10⁶ cells/kg, from 1×10⁶ to 7×10⁶ cells/kg, from 1×10⁶ to 6×10⁶ cells/kg, from 1×10⁶ to 5×10⁶ cells/kg, from 1×10⁶ to 4×10⁶ cells/kg, from 1×10⁶ to 3×10⁶ cells/kg, from 1×10⁶ to 2×10⁶ cells/kg, from 2×10⁶ to 1×10⁸ cells/kg, from 2×10⁶ to 9×10⁷ cells/kg, from 2×10⁶ to 6×10⁷ cells/kg, from 2×10⁶ to 3×10⁷ cells/kg, from 2×10⁶ to 1×10⁷ cells/kg, from 2×10⁶ to 9×10⁶ cells/kg, from 2×10⁶ to 8×10⁶ cells/kg, from 2×10⁶ to 7×10⁶ cells/kg, from 2×10⁶ to 6×10⁶ cells/kg, from 2×10⁶ to 5×10⁶ cells/kg, from 2×10⁶ to 4×10⁶ cells/kg, from 2×10⁶ to 3×10⁶ cells/kg, from 3×10⁶ to 1×10⁸ cells/kg, from 3×10⁶ to 9×10⁷ cells/kg, from 3×10⁶ to 6×10⁷ cells/kg, from 3×10⁶ to 3×10⁷ cells/kg, from 3×10⁶ to 1×10⁷ cells/kg, from 3×10⁶ to 9×10⁶ cells/kg, from 3×10⁶ to 8×10⁶ cells/kg, from 3×10⁶ to 7×10⁶ cells/kg, from 3×10⁶ to 6×10⁶ cells/kg, from 3×10⁶ to 5×10⁶ cells/kg, from 3×10⁶ to 4×10⁶ cells/kg, from 4×10⁶ to 1×10⁸ cells/kg, from 4×10⁶ to 9×10⁷ cells/kg, from 4×10⁶ to 6×10⁷ cells/kg, from 4×10⁶ to 3×10⁷ cells/kg, from 4×10⁶ to 1×10⁷ cells/kg, from 4×10⁶ to 9×10⁶ cells/kg, from 4×10⁶ to 8×10⁶ cells/kg, from 4×10⁶ to 7×10⁶ cells/kg, from 4×10⁶ to 6×10⁶ cells/kg, from 4×10⁶ to 5×10⁶ cells/kg, from 5×10⁶ to 1×10⁸ cells/kg, from 5×10⁶ to 9×10⁷ cells/kg, from 5×10⁶ to 6×10⁷ cells/kg, from 5×10⁶ to 3×10⁷ cells/kg, from 5×10⁶ to 1×10⁷ cells/kg, from 5×10⁶ to 9×10⁶ cells/kg, from 5×10⁶ to 8×10⁶ cells/kg, from 5×10⁶ to 7×10⁶ cells/kg, from 5×10⁶ to 6×10⁶ cells/kg, from 6×10⁶ to 1×10⁸ cells/kg, from 6×10⁶ to 9×10⁷ cells/kg, from 6×10⁶ to 6×10⁷ cells/kg, from 6×10⁶ to 3×10⁷ cells/kg, from 6×10⁶ to 1×10⁷ cells/kg, from 6×10⁶ to 9×10⁶ cells/kg, from 6×10⁶ to 8×10⁶ cells/kg, from 6×10⁶ to 7×10⁶ cells/kg, from 7×10⁶ to 1×10⁸ cells/kg, from 7×10⁶ to 9×10⁷ cells/kg, from 7×10⁶ to 6×10⁷ cells/kg, from 7×10⁶ to 3×10⁷ cells/kg, from 7×10⁶ to 1×10⁷ cells/kg, from 7×10⁶ to 9×10⁶ cells/kg, from 7×10⁶ to 8×10⁶ cells/kg, from 8×10⁶ to 1×10⁸ cells/kg, from 8×10⁶ to 9×10⁷ cells/kg, from 8×10⁶ to 6×10⁷ cells/kg, from 8×10⁶ to 3×10⁷ cells/kg, from 8×10⁶ to 1×10⁷ cells/kg, from 8×10⁶ to 9×10⁶ cells/kg, from 9×10⁶ to 1×10⁸ cells/kg, from 9×10⁶ to 9×10⁷ cells/kg, from 9×10⁶ to 6×10⁷ cells/kg, from 9×10⁶ to 3×10⁷ cells/kg, from 9×10⁶ to 1×10⁷ cells/kg, from 1×10⁷ to 1×10⁸ cells/kg, from 1×10⁷ to 9×10⁷ cells/kg, from 1×10⁷ to 6×10⁷ cells/kg, from 1×10⁷ to 3×10⁷ cells/kg, from 3×10⁷ to 1×10⁸ cells/kg, from 3×10⁷ to 9×10⁷ cells/kg, from 3×10⁷ to 6×10⁷ cells/kg, from 6×10⁷ to 1×10⁸ cells/kg, from 6×10⁷ to 9×10⁷ cells/kg, or from 9×10⁷ to 1×10⁸ cells/kg. In certain embodiments, the effective amount of immune cells is from 1×10⁶ to 1×10⁷ cells/kg.

In certain embodiments, the effective amount of immune cells is at least 1×10⁵ cells/kg. For example, in certain embodiments, the effective amount of immune cells is at least 1×10⁸ cells/kg, at least 9×10⁷ cells/kg, at least 6×10⁷ cells/kg, at least 3×10⁷ cells/kg, at least 1×10⁷ cells/kg, at least 9×10⁶ cells/kg, at least 8×10⁶ cells/kg, at least 7×10⁶ cells/kg, at least 6×10⁶ cells/kg, at least 5×10⁶ cells/kg, at least 4×10⁶ cells/kg, at least 3×10⁶ cells/kg, at least 2×10⁶ cells/kg, at least 1×10⁶ cells/kg, at least 9×10⁵ cells/kg, at least 6×10⁵ cells/kg, at least 3×10⁵ cells/kg, or at least 1×10⁵ cells/kg. In certain embodiments, the effective amount of immune cells is at least 3×10⁵ cells/kg, at least 1×10⁶ cells/kg, at least 3×10⁶ cells/kg, or at least 1×10⁷ cells/kg. In certain embodiments, the effective amount of immune cells is at least 1×10⁶ cells/kg. In certain embodiments, the effective amount of immune cells is 1×10⁸ cells/kg, 9×10⁷ cells/kg, 6×10⁷ cells/kg, 3×10⁷ cells/kg, 1×10⁷ cells/kg, 9×10⁶ cells/kg, 8×10⁶ cells/kg, 7×10⁶ cells/kg, 6×10⁶ cells/kg, 5×10⁶ cells/kg, 4×10⁶ cells/kg, 3×10⁶ cells/kg, 2×10⁶ cells/kg, 1×10⁶ cells/kg, 9×10⁵ cells/kg, 6×10⁵ cells/kg, 3×10⁵ cells/kg, or 1×10⁵ cells/kg. In certain embodiments, the effective amount of immune cells is 3×10⁵ cells/kg, 1×10⁶ cells/kg, 3×10⁶ cells/kg, or 1×10⁷ cells/kg. In certain embodiments, the effective amount of immune cells is 1×10⁶ cells/kg. In certain embodiments, the effective amount of immune cells is 1×10⁷ cells/kg.

In certain embodiments, the effective amount of immune cells is administered to the subject in a single dose. In certain embodiments, the effective amount of immune cells is administered to the subject at least 1, at least 2, at least 3, at least 4, at least 5, at least 6, or at least 7 days after completing administration of the preconditioning regimen. In certain embodiments, the effective amount of immune cells is administered intravenously.

The disclosure further relates to pharmaceutical compositions comprising an effective amount of immune cells (e.g., T cells, e.g., CAR T cells) as described herein. In certain embodiments, the pharmaceutical compositions further comprise a pharmaceutically acceptable carrier.

c. Therapeutic Uses

The present disclosure provides methods for decreasing an unwanted immune response in a subject by (1) administering to the subject a preconditioning regimen as described herein (e.g., a preconditioning regimen summarized in TABLE 1), and (2) administering an effective amount of engineered immune cells (e.g., CAR immune cells, e.g., CAR T cells), wherein the immune cells target and kill a target immune cell (e.g., a B cell), thereby reducing the unwanted immune response in the subject.

In certain instances, unwanted immune responses are mediated, at least in part, by the activity of immunoglobulins. Immunoglobulins are glycoproteins belonging to the immunoglobulin superfamily which recognize antigens and facilitate the humoral response of the immune system. Immunoglobulins may occur in two physical forms, a soluble form that is secreted from the cell, and a membrane-bound form that is attached to the surface of a B cell and is referred to as the B cell receptor (BCR). Immature B cells, which have not been exposed to an antigen, are known as naive B cells and express only the IgM isotype in a cell surface-bound form. B cells begin to express both IgM and IgD when they reach maturity, indicating that they are ready to respond to antigen. B cell activation follows engagement of the BCR with an antigen, causing the cell to divide and differentiate into an antibody-producing plasma cell. In this activated form, the B cell starts to produce antibody in a secreted form rather than a membrane-bound form. However, B cells producing antibodies directed against self antigens (autoantibodies) can lead to an unwanted immune response against an organism's own cells and tissues, thereby contributing to an autoimmune disorder.

Thus, the disclosure provides a method of treating an autoimmune disease in a subject. The method comprises administering to the subject a preconditioning regimen described herein (e.g., a preconditioning regimen set forth in TABLE 1), and administering to the subject an effective amount of engineered immune cells (e.g., CAR immune cells, e.g., CAR T cells) which target an immune cell, e.g., which target a B cell. In certain embodiments, the method is used to decrease the number of circulating B cells in a subject. In certain embodiments, B cells in the subject produce autoantibodies having specificity for self antigens, e.g., anti-dsDNA antibodies. In certain embodiments, the method is used to decrease the number of antibodies, e.g., autoantibodies, in a subject. In certain embodiments, the autoimmune disease is a B-cell-mediated autoimmune disease, i.e., an autoimmune disease wherein (1) at least some symptoms or manifestations of the disease are attributable to, and/or exacerbated by, immunoglobulins produced by the subject's B cells, and/or (2) B cells have some role in initiating or maintaining the disease or a symptom or manifestation thereof.

Examples of B-cell-mediated autoimmune diseases include, but are not limited to: systemic lupus erythematosus (SLE), lupus nephritis, SLE with anti-dsDNA antibodies, pemphigus vulgaris (PV), mucosal PV, mucocutaneous PV, myasthenia gravis (MG), MuSK-associated MG, AChR MG, myositis, membranous nephropathy, anti-synthetase syndrome, dermatomyositis, immune mediated necrotizing myopathy, multiple sclerosis, neuromyelitis optica, chronic inflammatory demyelinating polyneuropathy, anti-NMDA Receptor encephalitis, Lambert-Eaton syndrome, pemphigus foliaceus, epidermolysis bullosa acquisita, bullous pemphigoid, Goodpasture's syndrome, rheumatoid arthritis, systemic sclerosis, anti-neutrophil cytoplasmic antibody (ANCA)-associated vasculitis, immune thrombocytopenic purpura, antiphospholipid syndrome, autoimmune hemolytic anemia, type 1 diabetes, Grave's disease, Hashimoto's disease, and Sjogren's syndrome. In certain embodiments, a therapeutic method described herein is used to treat any of the foregoing autoimmune diseases in a subject. In certain embodiments, a therapeutic method described herein is used to treat SLE, lupus nephritis, SLE with anti-dsDNA antibodies, PV, mucosal PV, mucocutaneous PV, MG, MuSK-associated MG, AChR MG, myositis, membranous nephropathy, anti-synthetase syndrome, dermatomyositis, or immune mediated necrotizing myopathy in a subject in need thereof. In certain embodiments, a therapeutic method described herein is used to treat SLE, lupus nephritis, SLE with anti-dsDNA antibodies, PV, mucosal PV, mucocutaneous PV, MG, MuSK-associated MG, AChR MG, myositis, membranous nephropathy, anti-synthetase syndrome, dermatomyositis, or immune mediated necrotizing myopathy in a subject in need thereof.

In certain embodiments, treating a subject using a method of the disclosure results in a decrease in autoantibody levels (e.g., anti-dsDNA antibody levels or anti-nuclear antibody levels) in the subject. For example, in certain embodiments, autoantibody levels in the subject decrease by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, or at least 99.9% e.g., as compared to autoantibody levels in the subject prior to administering the treatment. In certain embodiments, treating a subject with a method of the disclosure makes autoantibody levels undetectable in the subject. Autoantibody levels can be measured using any appropriate technique known in the art including, for example, by ELISA.

In certain embodiments, treating a subject using a method of the disclosure results in a decrease in circulating B cell levels. For example, in certain embodiments, B cell levels in the subject decrease by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, or at least 99.9%, e.g., as compared to levels of circulating B cells in the subject prior to administering the treatment. In certain embodiments, treating a subject with a method of the disclosure makes circulating B cell levels undetectable in the subject. In certain embodiments, the decrease in circulating B cells is transient, whereby B cell levels are restored partially or completely after a period of time.

In certain embodiments, treating a subject using a method of the disclosure results in a decrease in the amount of circulating CD19+ cells. For example, in certain embodiments, the amount of circulating CD19+ cells in the subject decreases by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, or at least 99.9%, e.g., as compared to levels of circulating B cells in the subject prior to administering the treatment. In certain embodiments, treating a subject with a method of the disclosure makes the amount of circulating CD19+ cells undetectable in the subject. In certain embodiments, the decrease in the amount of circulating CD19+ cells is transient, and levels are restored partially or completely after a period of time.

In certain embodiments, therapeutic methods of the disclosure may be used to treat SLE in a subject.

In certain embodiments, treating lupus nephritis in a subject using a method of the disclosure results in a reduction in proteinuria, e.g., as measured by urine protein concentration. For example, in certain embodiments, proteinuria in the subject is reduced by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, or at least 99.9%, e.g., as compared to proteinuria in the subject prior to administering the treatment or as compared to an appropriate control subject or group that did not receive the treatment. In certain embodiments, treating a subject with a method of the disclosure eliminates proteinuria.

In certain embodiments, treating SLE in a subject using a method of the disclosure results in an increase in the amount or activity of one or more complement factors, e.g., as detected by measuring serum levels of complement component 3 (C3) or complement component 4 (C4), or by measuring the 50% hemolytic complement (CH50) activity. For example, in certain embodiments, the amount of a complement factor in a subject is increased by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, at least 95%, at least 100%, at least 125%, at least 150%, at least 200%, at least 300%, at least 400%, at least 500%, or at least 1,000%, e.g., as compared to the amount of the complement factor in the subject prior to administering the treatment, or as compared to an appropriate control subject or group that did not receive the treatment. In certain embodiments, the CH50 activity in a subject is increased by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, at least 95%, at least 100%, at least 125%, at least 150%, at least 200%, at least 300%, at least 400%, or at least 500%, e.g., as compared to the CH50 activity in the subject prior to administering the treatment, or as compared to an appropriate control subject or group that did not receive the treatment.

In certain embodiments, treating an autoimmune disease in a subject using a method of the disclosure results in a decrease in a disease symptom or disease activity. For example, in certain embodiments, treating SLE (e.g., lupus nephritis) in a subject using a method of the disclosure results in a decrease in disease activity as measured by an SLE disease activity index or scale, such as the SLEDAI-2K responder index, the British Isles Lupus Assessment Group (BILAG) index, the Physician Global Assessment scale, the Cutaneous LE Disease Area and Severity Index (CLASI), the SLICC Damage Index (SDI), and/or the count of tender and swollen joints. In certain embodiments following treatment of the subject for SLE, the subject meets the complete renal remission (CRR) criteria, the SRI-4 responder criteria, the SRI-5 responder criteria, the SRI-6 responder criteria, the BICLA responder criteria, the LLDAS criteria, and/or the DORIS remission criteria.

In certain embodiments wherein a therapeutic method of the disclosure is used to treat SLE in a subject in need thereof, the subject's SLEDAI-2K score is decreased. For example, in certain embodiments, the subject's SLEDAI-2K score is decreased by at least 1, at least 2, at least 3, at least 4, at least 5, at least 6, at least 7, at least 8, at least 9, at least 10, at least 12, at least 14, at least 16, at least 18, at least 20, at least 25, or at least 30, e.g., as compared to the subject's score prior to being administered the treatment, or as compared to an appropriate control subject or group that did not receive the treatment. In certain embodiments, the subject's SLEDAI-2K score is reduced to 0-10, 0-9, 0-8, 0-7, 0-6, 0-5, 0-4, 0-3, 0-2, or 0-1. In certain embodiments, the subject's SLEDAI-2K score is reduced to 5, 4, 3, 2, 1, or 0. In certain embodiments, the subject's SLEDAI-2K score is reduced to 0.

In certain embodiments wherein a therapeutic method of the disclosure is used to treat SLE in a subject in need thereof, the subject's BILAG-2004 score decreases. For example, in certain embodiments, the subject's BILAG score decreases by at least 1 grade, by at least 2 grades, or by 3 grades, e.g., as compared to the subject's BILAG score prior to being treated according to a method of the disclosure, or as compared to an appropriate control subject or group that did not receive the treatment. In certain embodiments, the subject's BILAG score decreases by 1, 2, or 3 grades. In certain embodiments, the subject's BILAG score decreases to grade B, to grade C, or to grade D.

In certain embodiments wherein a therapeutic method of the disclosure is used to treat SLE in a subject in need thereof, the subject's PGA score decreases after being treated in accordance with a method of disclosure. For example, in certain embodiments, the subject's PGA score decreases by at least 0.1, at least 0.2, at least 0.3, at least 0.4, at least 0.5, at least 0.6, at least 0.8, at least 1, at least 1.5, at least 2, or at least 2.5 e.g., as compared to the subject's PGA score prior to being treated according to a method of the disclosure, or as compared to an appropriate control subject or group that did not receive the treatment.

In certain embodiments wherein a therapeutic method of the disclosure is used to treat SLE in a subject in need thereof, the subject's CLASI score decreases after being treated in accordance with a method of disclosure. For example, in certain embodiments, the subject's CLASI score decreases by at least 1, at least 2, at least 3, at least 4, at least 5, at least 6, at least 7, at least 8, at least 9, at least 10, at least 11, at least 12, at least 13, at least 14, at least 15, at least 20, at least 25, or at least 30, e.g., as compared to the subject's CLASI score prior to being treated according to a method of the disclosure, or as compared to an appropriate control subject or group that did not receive the treatment.

In certain embodiments, therapeutic methods of the disclosure may be used to treat myositis (idiopathic inflammatory myopathies, e.g., dermatomyositis (DM), anti-synthetase syndrome (ASyS), and immune-mediated necrotizing myopathy (IMNM)) in a subject. In certain embodiments, treating myositis in a subject using a method of the disclosure results in a decrease in a disease symptom or activity as measured by an appropriate index, assessment, or scale, e.g., any of the following: MMT-8, Patient Global Assessment of Disease Activity (PGA), Physician Global Assessment of Disease Activity (MDGA), Health Assessment Questionnaire Disability Index (HAQ-DI), Extramuscular Global Assessment (Myositis Disease Activity Assessment Tool [MDAAT]), and Pain and Fatigue Numeric Rating Scale (NRS).

In certain embodiments, treating myositis in a subject using a method of the disclosure results in a decrease in the serum level of an enzyme in the subject, e.g., creatine kinase (CK), alanine aminotransferase (ALT), aspartate aminotransferase (AST), aldolase, myoglobin, or lactate dehydrogenase (LDH). For example, in certain embodiments, the serum level of the enzyme in the subject is reduced by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, or at least 95%, e.g., as compared to the serum level of the enzyme in the subject prior to administering the treatment, or as compared to an appropriate control subject or group that did not receive the treatment.

In certain embodiments, treating a subject using a method of the disclosure results in a decrease in myositis-specific antibody (MSA) levels in the subject. For example, in certain embodiments, MSA levels in the subject decrease by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, or at least 99.9% e.g., as compared to MSA levels in the subject prior to administering the treatment. In certain embodiments, treating a subject with a method of the disclosure makes MSA levels undetectable in the subject.

In certain embodiments, therapeutic methods of the disclosure may be used to treat pemphigus vulgaris in a subject. In certain embodiments, treating pemphigus vulgaris in a subject using a method of the disclosure results in a decrease in a disease symptom or disease activity as measured by an appropriate index, assessment, or scale, e.g., the Pemphigus Disease Activity Index (PDAI). For example, in certain embodiments, the PDAI score of the subject is reduced by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, or at least 95%, e.g., as compared to the score of the subject prior to administering the treatment, or as compared to an appropriate control subject or group that did not receive the treatment.

In certain embodiments, therapeutic methods of the disclosure may be used to treat systemic sclerosis in a subject. In certain embodiments, treating systemic sclerosis in a subject using a method of the disclosure results in a decrease in a disease symptom or activity as measured by an appropriate index, assessment, or scale, e.g., any of the following: Composite Response Index for Systemic Sclerosis (CRISS), forced vital capacity (FVC), modified Rodnan Skin Score (mRSS), Health Assessment Questionnaire Disability Index (HAQ-DI), Patient Global Assessment (PtGA), Physician Global Assessment (PGA), Scleroderma Health Assessment Questionnaire (SHAQ), Scleroderma Skin Patient Reported Outcome (SSPRO), University of California Los Angeles Scleroderma Clinical Trials Consortium Gastrointestinal Tract Instrument (UCLA SCTC GIT), Functional Assessment of Chronic Illness Therapy-Fatigue (FACIT-F), Pain Numeric Rating Scale (NRS), 36-Item Short Form Survey (SF-36), and EuroQol-5 Dimensions-5 Levels (EQ-5D-5L).

In certain embodiments, therapeutic methods of the disclosure may be used to treat myasthenia gravis in a subject. In certain embodiments, treating myasthenia gravis in a subject using a method of the disclosure results in a decrease in a disease symptom or activity as measured by an appropriate index, assessment, or scale, e.g., any of the following: Myasthenia Gravis—Activities of Daily Living (MG-ADL), Quantitative Myasthenia Gravis (QMG), Myasthenia Gravis Composite (MGC), Myasthenia Gravis Foundation of America Post-intervention Status (MGFA-PIS), MG Impairment Index (MGII), 15-item Myasthenia Gravis Quality of Life (MG-QOL 15r), Quality of Life in Neurological Disorders (Neuro-QoL), and EQ-5D-5L.

In certain embodiments, treating a subject having myasthenia gravis using a method of the disclosure results in a decrease in autoantibody levels (e.g., anti-acetylcholine receptor (anti-AChR), anti-muscle-specific kinase (anti-MuSK), and/or anti-low-density lipoprotein receptor-related protein 4 (anti-LRP4) autoantibodies) in the subject. For example, in certain embodiments, levels of anti-AChR, anti-MuSK, and/or anti-LRP4 autoantibodies in the subject decrease by at least 1%, at least 2%, at least 3%, at least 4%, at least 5%, at least 6%, at least 8%, at least 10%, at least 15%, at least 20%, at least 25%, at least 30%, at least 40%, at least 50%, at least 60%, at least 70%, at least 80%, at least 90%, at least 95%, at least 96%, at least 97%, at least 98%, at least 99%, at least 99.5%, at least 99.6%, at least 99.7%, at least 99.8%, or at least 99.9%, e.g., as compared to autoantibody levels in the subject prior to administering the treatment. In certain embodiments, treating a subject with a method of the disclosure makes autoantibody levels undetectable in the subject. Autoantibody levels can be measured using any appropriate technique known in the art including, for example, by ELISA.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein is administered in combination with one or more additional therapies. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodilator, a statin, an anti-inflammatory agent (e.g. methotrexate), or an NSAID. In certain embodiments, the additional therapy may include a combination of therapeutics of different classes. In certain embodiments, the additional therapy may be an antibody therapy (e.g., belimumab) or a corticosteroid.

The disclosure provides a method of conditioning a subject for immune cell therapy (e.g., a CAR immune cell therapy for the treatment of an autoimmune disease), the method comprising administering to the subject a preconditioning regimen comprising a dose of cyclophosphamide and a dose of fludarabine as described herein. In a related aspect, the disclosure provides a method of enhancing the effectiveness of an immune cell therapy (e.g., a CAR immune cell therapy for the treatment of an autoimmune disease) by conditioning a subject by administering to the patient a preconditioning regimen comprising a dose of cyclophosphamide and a dose of fludarabine as described herein. Without wishing to be bound by theory, it is understood that the preconditioning regimens of the disclosure enhance the survival, persistence, and efficacy of the administered CAR immune cells, for example, by increasing levels of homeostatic cytokines such as IL-7 and IL-15, and by reducing the number of endogenous immune cells competing for said cytokines, thereby creating a more favorable environment for the infused cells to proliferate. It is believed that the preconditioning regimens of the disclosure effectively enable treatment of autoimmune disorders with cell therapy, while minimizing the toxic side effects and risks associated with cyclophosphamide and fludarabine treatment. In certain embodiments, the preconditioning regimens of the disclosure cause a lesser drop in white blood cell count when administered to a subject, e.g., as compared to a standard preconditioning regimen (e.g., 1,000 mg/m² cyclophosphamide and 25 mg/m² fludarabine).

### III. Chimeric Antigen Receptors

Exemplary CARs for use in the therapeutic methods of the disclosure are described in greater detail below, in addition to nucleic acids encoding such CARs, vectors comprising said nucleic acids, genetically engineered cells modified to express said CARs, and other related methods and compositions.

In certain embodiments, a CAR of the disclosure comprises (1) an extracellular domain comprising an antigen-binding site that provides specificity for a desired antigen (e.g., an immune cell surface antigen), (2) a transmembrane domain, (3) an intracellular signaling domain and, optionally, (4) a costimulatory domain. In certain embodiments, the intracellular signaling domain is, or is derived from, a stimulatory molecule, such as a T cell activating domain providing a primary activation signal. Upon specific binding to the targeted antigen, the receptor generally delivers an immunostimulatory signal, such as an ITAM-transduced signal, into the cell, thereby activating the cell and promoting a targeted immune response. In certain embodiments, the CAR further comprises one or more costimulatory signaling domains comprising functional signaling domains derived from one or more costimulatory molecules. Further examples of CARs are provided in U.S. Pat. Nos. 7,446,190 and 9,181,527, U.S. Patent Application Publication Nos. 2016/0340406 and 2017/0049819, and International Patent Application Publication No. WO2018/140725.

a. CAR Extracellular Domain

In certain embodiments, the extracellular domain of the CAR comprises an antigen-binding site that specifically binds a target antigen. For example, in certain embodiments, the extracellular antigen-binding domain comprises an antigen-binding fragment of an antibody or a derivative thereof. In certain embodiments, the extracellular domain comprises a Fab fragment or an scFv. In certain embodiments, the extracellular domain comprises an scFv. In certain embodiments, the antigen binding site is present in the Fab or the scFv.

In certain embodiments, the target antigen specifically bound by the antigen binding site is a polypeptide. In certain embodiments, the target antigen is selectively expressed or overexpressed on cells of a particular cell type. In certain embodiments, the antigen is expressed by an immune cell. In certain embodiments, the targeted antigen is expressed by an immune cell, e.g., a B cell. In certain embodiments, the targeted antigen is present on the surface of an immune cell, e.g., a B cell. In certain embodiments, the antigen is a B cell marker. In certain embodiments, the antigen targeted by the CAR is CD19, CD20, BCMA, CD22, ROR1, CD45, CD21, CD5, CD33, Ig kappa, Ig lambda, CD79a, CD79b or CD30. In certain embodiments, the antigen is CD19.

As described herein, an antigen-binding site of a CAR of the disclosure may comprise: (a) an immunoglobulin heavy chain variable region (V_{H}) comprising the structure CDR_{H1}-CDR_{H2}-CDR_{H3} and (b) an immunoglobulin light chain variable region (V_{L}) comprising the structure CDR_{L1}-CDR_{L2}-CDR_{L3}, wherein the V_{H} and the V_{L} together define a single binding site for binding a targeted antigen. In certain embodiments, the V_{H} and V_{L} each comprises one or more framework (FR) regions (e.g., 1, 2, 3, or 4 framework regions). In certain embodiments, the V_{H} comprises the structure FR_{H1}-CDR_{H1}-FR_{H2}-CDR_{H2}-FR_{H3}-CDR_{H3}-FR_{H4} and/or the V_{L} comprises the structure FR_{L1}-CDR_{L1}-FR_{L2}-CDR_{L2}-FR_{L3}-CDR_{L}-FR_{L4}.

Exemplary antigen-binding sites that bind CD19 and that can be used in a CAR of the disclosure are described in greater detail hereinbelow.

In certain embodiments, the antigen-binding site that binds CD19 comprises a V_{H} that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the V_{H} of an antigen-binding site disclosed in TABLE 2, 3, or 4 and a V_{L} that comprises an amino acid sequence at least 60% (e.g., at least 70%, at least 80%, at least 85%, at least 90%, at least 91%, at least 92%, at least 93%, at least 94%, at least 95%, at least 96%, at least 97%, at least 98%, at least 99%, or 100%) identical to the V_{L} of the same antigen-binding site disclosed in TABLE 2, 3 or 4. In certain embodiments, the antigen-binding site that binds CD19 comprises CDR_{H1}, CDR_{H2}, CDR_{H3}, CDR_{L1}, CDR_{L2}, and CDR_{L3} sequences present in the V_{H} sequence of SEQ ID NO: 4 and the V_{L} sequence of SEQ ID NO: 8, which can be identified using CDR determination algorithms known in the art, for example, the algorithms disclosed herein. In certain embodiments, the antigen-binding site that binds CD19 comprises CDR_{H1}, CDR_{H2}, CDR_{H3}, CDR_{L1}, CDR_{L2}, and CDR_{L3} sequences present in the V_{H} sequence of SEQ ID NO: 13 and the V_{L} sequence of SEQ ID NO: 17, which can be identified using CDR determination algorithms known in the art, for example, the algorithms disclosed herein. In certain embodiments, the antigen-binding site that binds CD19 comprises CDR_{H1}, CDR_{L2}, CDR_{H3}, CDR_{L1}, CDR_{L2}, and CDR_{L3} sequences present in the V_{H} sequence of SEQ ID NO: 44 and the V_{L} sequence of SEQ ID NO: 48, which can be identified using CDR determination algorithms known in the art, for example, the algorithms disclosed herein. In certain embodiments, the antigen-binding site comprises CDR_{H1}, CDR_{H2}, CDR_{H3}, CDR_{L1}, CDR_{L2}, and CDR_{L3} sequences determined under IMGT (see Lefranc, (1999) THE IMMUNOLOGIST, 7, 132-136), e.g., as indicated in TABLE 2, where the CDR sequences in each of the V_{H} and V_{L} sequences are underlined and then denoted individually. In certain embodiments, the antigen-binding site comprises CDR_{H1}, CDR_{H2}, CDR_{H3}, CDR_{L1}, CDR_{L2}, and CDR_{L3} sequences determined under Chothia (see, e.g., Chothia C & Lesk A M, (1987), J. MOL. BIOL. 196: 901-917), e.g., as indicated in TABLE 3, where the CDR sequences in each of the V_{H} and V_{L} sequences are underlined and then denoted individually. In certain embodiments, the antigen-binding site comprises CDR_{H1}, CDR_{H2}, CDR_{H3}, CDR_{L1}, CDR_{L2}, and CDR_{L3} sequences determined under Kabat (see Kabat et al., (1991) Sequences of Proteins of Immunological Interest, NIH Publication No. 91-3242, Bethesda), e.g., as indicated in TABLE 4, where the CDR sequences in each of the V_{H} and V_{L} sequences are underlined and then denoted individually. In certain embodiments, the antigen-binding site comprises CDR_{H1}, CDR_{H2}, CDR_{H3}, CDR_{L1}, CDR_{L2}, and CDR_{L3} sequences determined under MacCallum (see, MacCallum R M et al., (1996) J. MOL. BIOL. 262: 732-745) or any other CDR determination method known in the art, of the V_{H} and V_{L} sequences of an antibody disclosed in TABLE 2, 3, or 4. Identification of CDR and framework sequences is within the level of ordinary skill in the art, and it is understood that the boundaries between CDR and framework sequences may depend upon the definition or convention that is used (e.g., IMGT, Kabat, Chothia, etc.).

In certain embodiments, the antigen-binding site comprises the CDR_{H1}, CDR_{H2}, CDR_{H3}, CDR_{L1}, CDR_{L2}, and CDR_{L3} sequences of an antigen-binding site disclosed in TABLE 2, where the CDR sequences in each of the V_{H} and V_{L} sequences are underlined. In certain embodiments, the antigen-binding site comprises the V_{H} and V_{L} sequences of an antigen-binding site disclosed in TABLE 2.

TABLE 2
Exemplary anti-CD19 antigen-binding sites (IMGT)

| Antigen | | |
|---|---|---|
| Binding | Heavy Chain Variable Domain | Light Chain Variable Domain |
| Site | (V_{H}) | (V_{L}) |
| CD19-1 | EVQLVQSGAEVKKPGESLKISC | QAVLTQPPSVSEAPRQRVTISCSGS |
| | KGSGYSFTSYWIGWVRQMPGK | SSNIGNNAVSWYQQLPGKAPKLLI |
| | GLEWMGIIYPGDSDTRYSPSFQG | YYDDLLPSGVSDRFSGSKSGTSAS |
| | QVTISADKSISTAYLQWSSLKAS | LAISGLQSEDEADYYCAAWDDSL |
| | DTAMYYCARLSYSWSSWYWDF | NGWVFGGGTKVTVLG (SEQ ID |
| | WGQGTLVTVSS (SEQ ID NO: 4) | NO: 8) |
| | CDR_{H1}-GYSFTSYW (SEQ ID | CDR_{L1}-SSNIGNNA (SEQ ID NO: 5) |
| | NO: 1) | CDR_{L2}-YDD (SEQ ID NO: 6) |
| | CDR_{H2}-YPGDSDT (SEQ ID NO: | CDR_{L3}-AAWDDSLNGWV (SEQ ID |
| | 2) | NO: 7) |
| | CDR_{H3}-ARLSYSWSSWYWDF | |
| | (SEQ ID NO: 3) | |
| CD19-2 | EVKLQESGPGLVAPSQSLSVTCT | DIQMTQTTSSLSASLGDRVTISCRA |
| | VSGVSLPDYGVSWIRQPPRKGL | SQDISKYLNWYQQKPDGTVKLLIY |
| | EWLGVIWGSETTYYNSALKSRL | HTSRLHSGVPSRFSGSGSGTDYSLT |
| | TIIKDNSKSQVFLKMNSLQTDDT | ISNLEQEDIATYFCQQGNTLPYTFG |
| | AIYYCAKHYYYGGSYAMDYW | GGTKLEIT (SEQ ID NO: 17) |
| | GQGTSVTVSS (SEQ ID NO: 13) | CDR_{L1}-QDISKY (SEQ ID NO: 14) |
| | CDR_{H1}-GVSLPDYG (SEQ ID | CDR_{L2}-HTS (SEQ ID NO: 15) |
| | NO: 10) | CDR_{L3}-QQGNTLPYT (SEQ ID NO: |
| | CDR_{H2}-IWGSETT (SEQ ID NO: | 16) |
| | 11) | |
| | CDR_{H3}-AKHYYYGGSYAMDY | |
| | (SEQ ID NO: 12) | |
| CD19-3 | QVQLVQSGAEVKKPGSSVKVSC | EIVLTQSPGTLSLSPGERATLSCRA |
| | KDSGGTFSSYAISWVRQAPGQG | SQSVSSSYLAWYQQKPGQAPRLLI |
| | LEWMGGIIPIFGTTNYAQQFQGR | YGASSRATGIPDRFSGSGSGTDFTL |
| | VTITADESTSTAYMELSSLRSED | TISRLEPEDFAVYYCQQYGSSRFTF |
| | TAVYYCAREAVAADWLDPWG | GPGTKVDIK (SEQ ID NO: 48) |
| | QGTLVTVSS (SEQ ID NO: 44) | CDR_{L1}-QSVSSSY (SEQ ID NO: 45) |
| | CDR_{H1}-GGTFSSYA (SEQ ID NO: | CDR_{L2}-GAS (SEQ ID NO: 46) |
| | 41) | CDR_{L3}-QQYGSSRFT (SEQ ID NO: |
| | CDR_{H2}-IIPIFGTT (SEQ ID NO: | 47) |
| | 42) | |
| | CDR_{H3}-AREAVAADWLDP (SEQ | |
| | ID NO: 43) | |

In certain embodiments, the antigen-binding site comprises the CDR_{H1}, CDR_{H2}, CDR_{H3}, CDR_{L1}, CDR_{L2}, and CDR_{L3} sequences of an antigen-binding site disclosed in TABLE 3, where the CDR sequences in each of the V_{H} and V_{L} sequences are underlined. In certain embodiments, the antigen-binding site comprises the V_{H} and V_{L} sequences of an antigen-binding site disclosed in TABLE 3.

TABLE 3
Exemplary anti-CD19 antigen-binding sites (Chothia)

| Antigen | | |
|---|---|---|
| Binding | Heavy Chain Variable Domain | Light Chain Variable Domain |
| Site | (V_{H}) | (V_{L}) |
| CD19-1 | EVQLVQSGAEVKKPGESLKISC | QAVLTQPPSVSEAPRQRVTISCSGSS |
| | KGSGYSFTSYWIGWVRQMPGK | SNIGNNAVSWYQQLPGKAPKLLIYY |
| | GLEWMGIIYPGDSDTRYSPSFQG | DDLLPSGVSDRFSGSKSGTSASLAIS |
| | QVTISADKSISTAYLQWSSLKAS | GLQSEDEADYYCAAWDDSLNGWV |
| | DTAMYYCARLSYSWSSWYWDF | FGGGTKVTVLG (SEQ ID NO: 8) |
| | WGQGTLVTVSS (SEQ ID NO: 4) | CDR_{L1}-SGSSSNIGNNAVS (SEQ ID |
| | CDR_{H1}-GYSFTSY (SEQ ID NO: | NO: 33) |
| | 30) | CDR_{L2}-YDDLLPS (SEQ ID NO: 34) |
| | CDR_{H2}-YPGDSD (SEQ ID NO: | CDR_{L3}-AAWDDSLNGWV (SEQ ID |
| | 31) | NO: 7) |
| | CDR_{H3}-LSYSWSSWYWDF (SEQ | |
| | ID NO: 32) | |
| CD19-2 | EVKLQESGPGLVAPSQSLSVTCT | DIQMTQTTSSLSASLGDRVTISCRAS |
| | VSGVSLPDYGVSWIRQPPRKGL | QDISKYLNWYQQKPDGTVKLLIYHT |
| | EWLGVIWGSETTYYNSALKSRL | SRLHSGVPSRFSGSGSGTDYSLTISN |
| | TIIKDNSKSQVFLKMNSLQTDDT | LEQEDIATYFCQQGNTLPYTFGGGT |
| | AIYYCAKHYYYGGSYAMDYW | KLEIT (SEQ ID NO: 17) |
| | GQGTSVTVSS (SEQ ID NO: 13) | CDR_{L1}-RASQDISKYLN (SEQ ID NO: |
| | CDR_{H1}-GVSLPDY (SEQ ID NO: | 39) |
| | 36 | CDR_{L2}-HTSRLHS (SEQ ID NO: 40) |
| | CDR_{H2}-WGSET (SEQ ID NO: 37) | CDR_{L3}-QQGNTLPYT (SEQ ID NO: |
| | CDR_{H3}-HYYYGGSYAMDY | 16) |
| | (SEQ ID NO: 38) | |
| CD19-3 | QVQLVQSGAEVKKPGSSVKVSC | EIVLTQSPGTLSLSPGERATLSCRAS |
| | KDSGGTFSSYAISWVRQAPGQG | QSVSSSYLAWYQQKPGQAPRLLIYG |
| | LEWMGGIIPIFGTTNYAQQFQGR | ASSRATGIPDRFSGSGSGTDFTLTISR |
| | VTITADESTSTAYMELSSLRSED | LEPEDFAVYYCQQYGSSRFTFGPGT |
| | TAVYYCAREAVAADWLDPWG | KVDIK (SEQ ID NO: 48) |
| | QGTLVTVSS (SEQ ID NO: 44) | CDR_{L1}-RASQSVSSSYLA (SEQ ID |
| | CDR_{H1}-GGTFSSY (SEQ ID NO: | NO: 52) |
| | 49) | CDR_{L2}-GASSRAT (SEQ ID NO: 53) |
| | CDR_{H2}-IPIFGT (SEQ ID NO: 50) | CDR_{L3}-QQYGSSRFT (SEQ ID NO: |
| | CDR_{H3}-EAVAADWLDP (SEQ ID | 47) |
| | NO: 51) | |

In certain embodiments, the antigen-binding site comprises the CDR_{H1}, CDR_{H2}, CDR_{H3}, CDR_{L1}, CDR_{L2}, and CDR_{L3} sequences of an antigen-binding site disclosed in TABLE 4, where the CDR sequences in each of the V_{H} and V_{L} sequences are underlined. In certain embodiments, the antigen-binding site comprises the V_{H} and V_{L} sequences of an antigen-binding site disclosed in TABLE 4.

TABLE 4
Exemplary anti-CD19 antigen-binding sites (Kabat)

| Antigen | | |
|---|---|---|
| Binding | Heavy Chain Variable Domain | Light Chain Variable Domain |
| Site | (V_{H}) | (V_{L}) |
| CD19-1 | EVQLVQSGAEVKKPGESLKISC | QAVLTQPPSVSEAPRQRVTISCSGSSS |
| | KGSGYSFTSYWIGWVRQMPGK | NIGNNAVSWYQQLPGKAPKLLIYYD |
| | GLEWMGIIYPGDSDTRYSPSFQ | DLLPSGVSDRFSGSKSGTSASLAISGL |
| | GQVTISADKSISTAYLQWSSLK | QSEDEADYYCAAWDDSLNGWVFGG |
| | ASDTAMYYCARLSYSWSSWY | GTKVTVLG (SEQ ID NO: 8) |
| | WDFWGQGTLVTVSS (SEQ ID | CDR_{L1}-SGSSSNIGNNAVS (SEQ ID |
| | NO: 4) | NO: 33) |
| | CDR_{H1}-SYWIG (SEQ ID NO: | CDR_{L2}-YDDLLPS (SEQ ID NO: 34) |
| | 54) | CDR_{L3}-AAWDDSLNGWV (SEQ ID |
| | CDR_{H2}-IIYPGDSDTRYSPSFQG | NO: 7) |
| | (SEQ ID NO: 55) | |
| | CDR_{H3}-LSYSWSSWYWDF | |
| | (SEQ ID NO: 32) | |
| CD19-2 | EVKLQESGPGLVAPSQSLSVTC | DIQMTQTTSSLSASLGDRVTISCRASQ |
| | TVSGVSLPDYGVSWIRQPPRKG | DISKYLNWYQQKPDGTVKLLIYHTS |
| | LEWLGVIWGSETTYYNSALKS | RLHSGVPSRFSGSGSGTDYSLTISNLE |
| | RLTIIKDNSKSQVFLKMNSLQT | QEDIATYFCQQGNTLPYTFGGGTKLE |
| | DDTAIYYCAKHYYYGGSYAM | IT (SEQ ID NO: 17) |
| | DYWGQGTSVTVSS (SEQ ID | CDR_{L1}-RASQDISKYLN (SEQ ID NO: |
| | NO: 13) | 39) |
| | CDR_{H1}-DYGVS (SEQ ID NO: | CDR_{L2}-HTSRLHS (SEQ ID NO: 40) |
| | 56) | CDR_{L3}-QQGNTLPYT (SEQ ID NO: |
| | CDR_{H2}-VIWGSETTYYNSALKS | 16) |
| | (SEQ ID NO: 57) | |
| | CDR_{H3}-HYYYGGSYAMDY | |
| | (SEQ ID NO: 38) | |
| CD19-3 | QVQLVQSGAEVKKPGSSVKVS | EIVLTQSPGTLSLSPGERATLSCRASQ |
| | CKDSGGTFSSYAISWVRQAPG | SVSSSYLAWYQQKPGQAPRLLIYGAS |
| | QGLEWMGGIIPIFGTTNYAQQF | SRATGIPDRFSGSGSGTDFTLTISRLEP |
| | QGRVTITADESTSTAYMELSSL | EDFAVYYCQQYGSSRFTFGPGTKVDI |
| | RSEDTAVYYCAREAVAADWL | K (SEQ ID NO: 48) |
| | DPWGQGTLVTVSS (SEQ ID | CDR_{L1}-RASQSVSSSYLA (SEQ ID |
| | NO: 44) | NO: 52) |
| | CDR_{H1}-SYAIS (SEQ ID NO: 58) | CDR_{L2}-GASSRAT (SEQ ID NO: 53) |
| | CDR_{H2}-GIIPIFGTTNYAQQFQG | CDR_{L3}-QQYGSSRFT (SEQ ID NO: 47) |
| | (SEQ ID NO: 59) | |
| | CDR_{H3}-EAVAADWLDP (SEQ | |
| | ID NO: 51) | |

In certain embodiments, the antigen-binding site that binds CD19 comprises a V_{H} comprising CDR_{H1}, CDR_{H2}, and CDR_{H3} sequences set forth in SEQ ID NOs: 1, 2, and 3, respectively, wherein CDR_{H1}, CDR_{H2}, and CDR_{H3} sequences are interposed between immunoglobulin FR sequences; and/or a V_{L} comprising CDR_{L1}, CDR_{L2}, and CDR_{L3} sequences set forth in SEQ ID NOs: 5, 6, and 7, respectively, wherein CDR_{L1}, CDR_{L2}, and CDR_{L3} sequences are interposed between immunoglobulin FR sequences. In certain embodiments, the CDR_{H1}, CDR_{H2}, and CDR_{H3} sequences and/or the CDR_{L1}, CDR_{L2}, and CDR_{L3} sequences are interposed between human or humanized immunoglobulin FR sequences.

In certain embodiments, the antigen-binding site that binds CD19 comprises a V_{H} comprising CDR_{H1}, CDR_{H2}, and CDR_{H3} sequences set forth in SEQ ID NOs: 10, 11, and 12, respectively, wherein CDR_{H1}, CDR_{H2}, and CDR_{H3} sequences are interposed between immunoglobulin FR sequences; and/or a V_{L} comprising CDR_{L1}, CDR_{L2}, and CDR_{L3} sequences set forth in SEQ ID NOs: 14, 15, and 16, respectively, wherein CDR_{L1}, CDR_{L2}, and CDR_{L3} sequences are interposed between immunoglobulin FR sequences. In certain embodiments, the CDR_{H1}, CDR_{H2}, and CDR_{H3} sequences and/or the CDR_{L1}, CDR_{L2}, and CDR_{L3} sequences are interposed between human or humanized immunoglobulin FR sequences.

In certain embodiments, the antigen-binding site that binds CD19 comprises a V_{H} comprising CDR_{H1}, CDR_{H2}, and CDR_{H3} sequences set forth in SEQ ID NOs: 41, 42, and 43, respectively, wherein CDR_{H1}, CDR_{H2}, and CDR_{H3} sequences are interposed between immunoglobulin FR sequences; and/or a V_{L} comprising CDR_{L1}, CDR_{L2}, and CDR_{L3} sequences set forth in SEQ ID NOs: 45, 46, and 47, respectively, wherein CDR_{L1}, CDR_{L2}, and CDR_{L3} sequences are interposed between immunoglobulin FR sequences. In certain embodiments, the CDR_{H1}, CDR_{H2}, and CDR_{H3} sequences and/or the CDR_{L1}, CDR_{L2}, and CDR_{L3} sequences are interposed between human or humanized immunoglobulin FR sequences.

In certain embodiments, the antigen-binding site that binds CD19 comprises a V_{H} comprising CDR_{H1}, CDR_{H2}, and CDR_{H3} sequences set forth in SEQ ID NOs: 30, 31, and 32, respectively, wherein CDR_{H1}, CDR_{H2}, and CDR_{H3} sequences are interposed between immunoglobulin FR sequences; and/or a V_{L} comprising CDR_{L1}, CDR_{L2}, and CDR_{L3} sequences set forth in SEQ ID NOs: 33, 34, and 7, respectively, wherein CDR_{L1}, CDR_{L2}, and CDR_{L3} sequences are interposed between immunoglobulin FR sequences. In certain embodiments, the CDR_{H1}, CDR_{H2}, and CDR_{H3} sequences and/or the CDR_{L1}, CDR_{L2}, and CDR_{L3} sequences are interposed between human or humanized immunoglobulin FR sequences.

In certain embodiments, the antigen-binding site that binds CD19 comprises a V_{H} comprising CDR_{H1}, CDR_{H2}, and CDR_{H3} sequences set forth in SEQ ID NOs: 36, 37, and 38, respectively, wherein CDR_{H1}, CDR_{H2}, and CDR_{H3} sequences are interposed between immunoglobulin FR sequences; and/or a V_{L} comprising CDR_{L1}, CDR_{L2}, and CDR_{L3} sequences set forth in SEQ ID NOs: 39, 40, and 16, respectively, wherein CDR_{L1}, CDR_{L2}, and CDR_{L3} sequences are interposed between immunoglobulin FR sequences. In certain embodiments, the CDR_{H1}, CDR_{H2}, and CDR_{H3} sequences and/or the CDR_{L1}, CDR_{L2}, and CDR_{L3} sequences are interposed between human or humanized immunoglobulin FR sequences.

In certain embodiments, the antigen-binding site that binds CD19 comprises a V_{H} comprising CDR_{H1}, CDR_{H2}, and CDR_{H3} sequences set forth in SEQ ID NOs: 49, 50, and 51, respectively, wherein CDR_{H1}, CDR_{H2}, and CDR_{H3} sequences are interposed between immunoglobulin FR sequences; and/or a V_{L} comprising CDR_{L1}, CDR_{L2}, and CDR_{L3} sequences set forth in SEQ ID NOs: 52, 53, and 47, respectively, wherein CDR_{L1}, CDR_{L2}, and CDR_{L3} sequences are interposed between immunoglobulin FR sequences. In certain embodiments, the CDR_{H1}, CDR_{H2}, and CDR_{H3} sequences and/or the CDR_{L1}, CDR_{L2}, and CDR_{L3} sequences are interposed between human or humanized immunoglobulin FR sequences.

In certain embodiments, the antigen-binding site that binds CD19 comprises a V_{H} comprising CDR_{H1}, CDR_{H2}, and CDR_{H3} sequences set forth in SEQ ID NOs: 54, 55, and 32, respectively, wherein CDR_{H1}, CDR_{H2}, and CDR_{H3} sequences are interposed between immunoglobulin FR sequences; and/or a V_{L} comprising CDR_{L1}, CDR_{L2}, and CDR_{L3} sequences set forth in SEQ ID NOs: 33, 34, and 7, respectively, wherein CDR_{L1}, CDR_{L2}, and CDR_{L3} sequences are interposed between immunoglobulin FR sequences. In certain embodiments, the CDR_{H1}, CDR_{H2}, and CDR_{H3} sequences and/or the CDR_{L1}, CDR_{L2}, and CDR_{L3} sequences are interposed between human or humanized immunoglobulin FR sequences.

In certain embodiments, the antigen-binding site that binds CD19 comprises a V_{H} comprising CDR_{H1}, CDR_{H2}, and CDR_{H3} sequences set forth in SEQ ID NOs: 56, 57, and 38, respectively, wherein CDR_{H1}, CDR_{H2}, and CDR_{H3} sequences are interposed between immunoglobulin FR sequences; and/or a V_{L} comprising CDR_{L1}, CDR_{L2}, and CDR_{L3} sequences set forth in SEQ ID NOs: 39, 40, and 16, respectively, wherein CDR_{L1}, CDR_{L2}, and CDR_{L3} sequences are interposed between immunoglobulin FR sequences. In certain embodiments, the CDR_{H1}, CDR_{H2}, and CDR_{H3} sequences and/or the CDR_{L1}, CDR_{L2}, and CDR_{L3} sequences are interposed between human or humanized immunoglobulin FR sequences.

In certain embodiments, the antigen-binding site that binds CD19 comprises a V_{H} comprising CDR_{H1}, CDR_{H2}, and CDR_{H3} sequences set forth in SEQ ID NOs: 58, 59, and 51, respectively, wherein CDR_{H1}, CDR_{H2}, and CDR_{H3} sequences are interposed between immunoglobulin FR sequences; and/or a V_{L} comprising CDR_{L1}, CDR_{L2}, and CDR_{L3} sequences set forth in SEQ ID NOs: 52, 53, and 47, respectively, wherein CDR_{L1}, CDR_{L2}, and CDR_{L3} sequences are interposed between immunoglobulin FR sequences. In certain embodiments, the CDR_{H1}, CDR_{H2}, and CDR_{H3} sequences and/or the CDR_{L1}, CDR_{L2}, and CDR_{L3} sequences are interposed between human or humanized immunoglobulin FR sequences.

In certain embodiments, the antigen-binding site that binds CD19 comprises a V_{H} comprising the amino acid sequence of SEQ ID NO: 4, SEQ ID NO: 13, or SEQ ID NO: 44. In certain embodiments, the antigen-binding site that binds CD19 comprises a V_{L} comprising the amino acid sequence of SEQ ID NO: 8, SEQ ID NO: 17, or SEQ ID NO: 48.

In certain embodiments, the antigen-binding site that binds CD19 comprises a V_{H} comprising the amino acid sequence of SEQ ID NO: 4, and/or a V_{L} comprising the amino acid sequence of SEQ ID NO: 8.

In certain embodiments, the antigen-binding site that binds CD19 comprises a V_{H} comprising the amino acid sequence of SEQ ID NO: 13, and/or a V_{L} comprising the amino acid sequence of SEQ ID NO: 17.

In certain embodiments, the antigen-binding site that binds CD19 comprises a V_{H} comprising the amino acid sequence of SEQ ID NO: 44, and/or a V_{L} comprising the amino acid sequence of SEQ ID NO: 48.

In certain embodiments, an antigen-binding site that binds CD19 comprises a V_{H} comprising an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to an amino acid sequence selected from SEQ ID NO: 4, SEQ ID NO: 13, and SEQ ID NO: 44. Alternatively or in addition, an antigen-binding site that binds CD19 comprises a V_{L} comprising an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to an amino acid sequence selected from SEQ ID NO: 8, SEQ ID NO: 17, and SEQ ID NO: 48.

In certain embodiments, an antigen-binding site that binds CD19 comprises a V_{H} comprising an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to the amino acid sequence of SEQ ID NO: 4; and/or comprises a V_{L} comprising an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to the amino acid sequence of SEQ ID NO: 8.

In certain embodiments, an antigen-binding site that binds CD19 comprises a V_{H} comprising an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to the amino acid sequence selected of SEQ ID NO: 13; and/or comprises a V_{L} comprising an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to the amino acid sequence of SEQ ID NO: 17.

In certain embodiments, an antigen-binding site that binds CD19 comprises a V_{H} comprising an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to the amino acid sequence selected of SEQ ID NO: 44; and/or comprises a V_{L} comprising an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to the amino acid sequence of SEQ ID NO: 48.

In each of the foregoing embodiments, it is contemplated herein that V_{H} sequences and/or V_{L} sequences that together bind CD19 may each independently contain amino acid alterations (e.g., at least 1, 2, 3, 4, 5, or 10 amino acid substitutions, deletions, or additions) in the framework regions of the V_{H} and/or the V_{L}. In certain embodiments, a V_{H} sequence and/or a V_{L} sequence that together bind CD19 may each independently contain 1-2, 1-3, 1-4, 1-5, 2-3, 2-4, 2-5, 3-4, 3-5, or 4-5 amino acid alterations (e.g., substitutions, deletions, or additions) in the framework regions of the V_{H} and/or the V_{L}. In certain embodiments, the framework regions of the V_{H} are humanized or human framework regions. In certain embodiments, the framework regions of the V_{L} are humanized or human framework regions.

In certain embodiments, an antigen-binding site that binds CD19 is present in an scFv. In certain embodiments, the scFv comprises the amino acid sequence of SEQ ID NO: 9. In certain embodiments, the scFv comprises the amino acid sequence of SEQ ID NO: 18. In certain embodiments, the scFv comprises the amino acid sequence of SEQ ID NO: 60.

In certain embodiments, an antigen-binding site that binds CD19 is present in an scFv, wherein the scFv comprises an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to the amino acid sequence of SEQ ID NO: 9.

In certain embodiments, an antigen-binding site that binds CD19 is present in an scFv, wherein the scFv comprises an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to the amino acid sequence selected of SEQ ID NO: 18.

In certain embodiments, an antigen-binding site that binds CD19 is present in an scFv, wherein the scFv comprises an amino acid sequence that is at least 60% (e.g., at least 70%, at least 75%, at least 80%, at least 85%, at least 86%, at least 87%, at least 88%, at least 89%, at least 89.5%, at least 90%, at least 90.5%, at least 91%, at least 92%, at least 93%, at least 94%, at least 94.5%, at least 95%, at least 95.5%, at least 96%, at least 97%, at least 98%, or at least 99%) identical to the amino acid sequence selected of SEQ ID NO: 60.

Further examples of antigen-binding sites that bind CD19 are provided in U.S. Pat. Nos. 7,446,179, 9,765,156, 10,125,193, 10,221,245, 10,287,350, 10,301,388, 10,457,730, 10,493,139, 10,533,055, 10,662,248, 10,780,118, 10,844,120, 10,874,693, 11,001,639, 11,034,750, 11,034,763, 11,077,144, and 11,141,436; U.S. Patent Application Publication Nos. 2020/0038443, 2020/0062843, 2020/0123254, 2020/0289563, 2020/0376033, 2020/0384023, 2020/0384026, 2021/0002366, 2021/0061907, 2021/0069244, 2021/0101978, 2021/0196756, 2021/0238253, 2021/0332133, 2021/0395362, and 2021/0395364; and International Patent Application Publication Nos. 2018/201794, 2019/137518, 2019/154313, 2019/214332, 2020/233589, 2021/170146, 2021/217130, 2021/223719, 2021/223720, 2021/225532, and 2022/012683.

In certain embodiments, the antigen-binding site binds CD19 with a K_{D} of 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, or 0.05 nM or stronger, as measured using standard binding assays, for example, surface plasmon resonance or bio-layer interferometry. In certain embodiments, the antigen-binding site binds CD19 with a K_{D} of from about 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.05 nM, from about 1 nM to about 0.075 nM, from about 1 nM to about 0.1 nM, from about 1 nM to about 0.5 nM, from about 0.5 nM to about 0.05 nM, from about 0.5 nM to about 0.075 nM, from about 0.5 nM to about 0.1 nM, from about 0.1 nM to about 0.05 nM, from about 0.1 nM to about 0.075 nM, or from about 0.075 nM to about 0.05 nM, or from about 0.05 nM to about 0.035 nM, as measured using standard binding assays, for example, surface plasmon resonance or bio-layer interferometry.

In certain embodiments, the antigen-binding site that binds CD19 cross-competes with an antigen-binding site disclosed in TABLE 2, 3, or 4. Competition assays for determining whether an antigen-binding site binds to the same epitope as, or competes for binding with a disclosed antibody are known in the art. Exemplary competition assays include immunoassays (e.g., ELISA assays, RIA assays), surface plasmon resonance, (e.g., BIAcore analysis), bio-layer interferometry, and flow cytometry.

The antigen-binding sites disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

Generally, an optimized antigen-binding site has at least the same, or substantially the same, affinity for the antigen as the non-optimized (or parental) antigen-binding site from which it was derived. Preferably, an optimized antibody has a higher affinity for the antigen when compared to the parental antibody.

The functional ability of a CAR to specifically bind to its target antigen (e.g., CD19) can be assessed in a Jurkat reporter cell line, wherein activation of the CAR is dependent on binding to plate-bound or cell-bound target protein (in response to which the activated cells fluoresce green due to an NFAT-GFP reporter construct contained therein). Such methods are useful and reliable qualitative measures for functional binding ability.

In certain embodiments, the extracellular binding domain of the CAR comprises means for binding CD19. In certain embodiments, the means is an antigen-binding site, e.g., an antigen-binding site that binds CD19 described herein.

b. CAR Transmembrane Domain

The extracellular antigen-binding domain can be fused to the transmembrane domain of the CAR. In certain embodiments, the transmembrane domain of the CAR is derived from a naturally occurring transmembrane protein. In certain embodiments, the transmembrane domain is capable of signaling to the intracellular domain(s) whenever the CAR has bound to a target. In some embodiments, the transmembrane domain comprises the transmembrane region(s) of one or more proteins selected from the group consisting of TCR α chain, TCR β chain, TCR ζ chain, CD28, CD3E, CD45, CD4, CD5, CD8α, CD9, CD16, CD22, EGFR, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C. In certain embodiments, the transmembrane comprises the transmembrane region(s) of one or more proteins selected from the group consisting of CD8α, CD28, CD3ζ, and CD4. In certain embodiments, the transmembrane comprises the transmembrane region(s) of one or more proteins selected from the group consisting of CD8α and CD28. In certain embodiments, the transmembrane domain comprises a CD8α transmembrane domain. In certain embodiments, the transmembrane domain is one that naturally is associated with one of the domains (e.g., primary signaling domain or co-stimulatory signaling domain) in the CAR. In certain embodiments, the transmembrane domain can be selected or modified by amino acid substitution to avoid multimerization with a transmembrane domain of the same or a different surface membrane protein, thereby to minimize interactions with other members of a receptor complex (e.g., the CAR complex). In other embodiments, the transmembrane domain is capable of homodimerization with another CAR on the immune cell (e.g., T cell) surface. In certain embodiments, the amino acid sequence of the transmembrane domain may be modified or substituted so as to minimize interactions with the binding domains of the native binding partner present in the same immune cell.

In certain embodiments, the transmembrane domain comprises a CD8α transmembrane domain. In certain embodiments, the transmembrane domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 19. In certain embodiments, the transmembrane domain comprises the amino acid sequence of SEQ ID NO: 19.

In certain embodiments, the transmembrane domain comprises a conservative substitution relative to a transmembrane domain disclosed herein, e.g., relative to a transmembrane domain comprising the amino acid sequence of SEQ ID NO: 19. In certain embodiments, the transmembrane domain comprises less than 2, less than 3, less than 4, less than 5, less than 6, less than 7, less than 8, less than 9, less than 10, less than 11, less than 12, less than 13, less than 14, or less than 15 conservative substitutions relative to, e.g., the amino acid sequence of SEQ ID NO: 19. In certain embodiments, the transmembrane domain comprises 1, 2, 3, 4, 5, 6, 7, 8, 9, 10, 11, 12, 13, 14, or 15 or more conservative substitutions relative to the amino acid sequence of SEQ ID NO: 19.

In certain embodiments, the transmembrane domain comprises a CD28 transmembrane domain. In certain embodiments, the transmembrane domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 61. In certain embodiments, the transmembrane domain comprises the amino acid sequence of SEQ ID NO: 61.

In certain embodiments, the transmembrane domain comprises a conservative substitution relative to a transmembrane domain disclosed herein, e.g., relative to a transmembrane domain comprising the amino acid sequence of SEQ ID NO: 61. In certain embodiments, the transmembrane domain comprises less than 2, less than 3, less than 4, less than 5, less than 6, less than 7, less than 8, less than 9, less than 10, less than 11, less than 12, less than 13, less than 14, or less than 15 conservative substitutions relative to, e.g., the amino acid sequence of SEQ ID NO: 61. In certain embodiments, the transmembrane domain comprises 1, 2, 3, 4, 5, 6, 7, 8, 9, 10, 11, 12, 13, 14, or 15 or more conservative substitutions relative to the amino acid sequence of SEQ ID NO: 61.

In certain embodiments, the CAR comprises a transmembrane domain and/or a cytoplasmic (intracellular) domain from a killer immunoglobulin-like receptor (KIR) family protein. The KIR gene family has at least 15 gene loci (KIR2DL1, KIR2DL2/L3, KIR2DL4, KIR2DL5A, KIR2DL5B, KIR2DS1, KIR2DS2, KIR2DS3, KIR2DS4, KIR2DS5 KIR3DL1/S1 KIR3DL2 KIR3DL3) and two pseudogenes (KIR2DP1 and KIR3DP1) encoded within a 100-200 Kb region of the Leukocyte Receptor Complex (LRC) located on chromosome 19 (19q13.4). The LRC constitutes a large, 1 Mb, and dense cluster of rapidly evolving immune genes which contains genes encoding other cell surface molecules with distinctive Ig-like extra-cellular domains. In addition, the extended LRC contains genes encoding the transmembrane adaptor molecules DAP10 and DAP12. Thus, in certain embodiments, a cell comprising the CAR of the disclosure comprising a KIR transmembrane domain and/or cytoplasmic domain may also comprise a polynucleotide encoding DAP10 or DAP12. In certain embodiments, the KIR is KIRS2 or KIR2DS2.

c. CAR Intracellular Domains

The intracellular domain of the CAR comprises an intracellular signaling domain (i.e., a functional signaling domain derived from a stimulatory molecule) and, optionally, one or more costimulatory signaling domains (i.e., functional signaling domains derived from at least one costimulatory molecule). These intracellular signaling and costimulatory domains are responsible, at least in part, for an immune cell response, including, but not limited to, proliferation, differentiation, and activation of a specialized function of the immune cell (e.g., cytotoxic activity or secretion of cytokines of a T cell) in which the CAR is expressed. The intracellular signaling domain is thus meant to include any truncated portion of the intracellular signaling domain sufficient to transduce the effector function signal.

Intracellular signaling domains that act in a stimulatory manner may contain signaling motifs which are known as immunoreceptor tyrosine-based activation motifs (ITAMs). Examples of ITAM-containing cytoplasmic signaling sequences that are of particular use in the CARs of the present application include those derived from CD3ζ, common FcRγ (FCER1G), FcγRIIa, FcRβ (FcεR1b), CD3γ, CD3δ, CD3ε, CD79a, CD79b, DAP10, and DAP12. In certain embodiments, the intracellular signaling domain of a CAR described herein comprises a functional, cytoplasmic signaling domain derived from CD3ζ, FcRγ, FcRβ, CD3γ, CD3δ, CD3ε, CD5, CD22, CD28, CD79a, CD79b, CD66d, 4-1BB, common FcRγ (FCER1G), FcγRIIa, FcRβ (FcεR1b), DAP10, and/or DAP12. In certain embodiments, the intracellular signaling domain comprises a CD3ζ signaling domain or an FcRγ signaling domain.

In certain embodiments, a CAR of the disclosure comprises a CD3ζ signaling domain by itself or in combination with any other desired cytoplasmic domain(s) useful in the context of the CAR. For example, the CAR can comprise a CD3ζ chain portion (i.e., a CD3ζ intracellular signaling domain) and an intracellular domain of a costimulatory molecule, for example, a 4-1BB intracellular domain. In certain embodiments, the CD3ζ intracellular signaling domain is a human T-cell surface glycoprotein CD3ζ chain isoform 3 (human CD247) intracellular domain. The human CD3ζ intracellular domain provides stimulatory intracellular signaling upon binding of the extracellular antigen to its ligand without HLA restriction.

In certain embodiments, a CAR of the disclosure comprises a CD3ζ signaling domain. In certain embodiments, the intracellular signaling domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 21. In certain embodiments, the intracellular signaling domain comprises the amino acid sequence of SEQ ID NO: 21.

In certain embodiments, the intracellular signaling domain comprises a conservative substitution relative to an intracellular signaling domain disclosed herein, e.g., relative to an intracellular signaling domain comprising the amino acid sequence of SEQ ID NO: 21. In certain embodiments, the intracellular signaling domain comprises less than 2, less than 3, less than 4, less than 5, less than 6, less than 7, less than 8, less than 9, less than 10, less than 11, less than 12, less than 13, less than 14, or less than 15 conservative substitutions relative to, e.g., the amino acid sequence of SEQ ID NO: 21. In certain embodiments, the intracellular signaling domain comprises 1, 2, 3, 4, 5, 6, 7, 8, 9, 10, 11, 12, 13, 14, or 15 or more conservative substitutions relative to the amino acid sequence of SEQ ID NO: 21.

In certain embodiments, a CAR of the disclosure comprises a costimulatory domain. A costimulatory domain comprises a functional signaling domain derived from a costimulatory molecule, a cell surface molecule other than an antigen receptor or its ligands that is required for an efficient response of lymphocytes to an antigen. Examples of costimulatory molecules include CD27, CD28, 4-1BB (CD137), OX40, CD30, CD40, PD-1, ICOS, lymphocyte function-associated antigen-1 (LFA-1, CD11a/CD18), CD2, CD7, CD258 (LIGHT), NKG2C, B7-H3, CD83 ligands, CD5, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, and PAG/Cbp. In certain embodiments, a costimulatory domain of the CAR comprises a functional signaling domain of a costimulatory molecule described herein, e.g., OX40, CD27, CD28, CD30, CD40, PD-1, CD2, CD7, CD258, NKG2C, B7-H3, a CD83 ligand, ICAM-1, LFA-1 (CD11a/CD18), ICOS and 4-1BB (CD137), or any combination thereof. In certain embodiments, a costimulatory domain of the CAR comprises a functional signaling domain of a costimulatory molecule selected from 4-1BB (CD137), CD28, ICOS, CD27, CD40, and OX40. In certain embodiments, the CAR comprises a costimulatory domain comprising a 4-1BB intracellular domain and/or a CD28 intracellular domain.

In certain embodiments, a CAR of the disclosure comprises a costimulatory domain comprising a 4-1BB intracellular domain. In certain embodiments, the costimulatory domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 20. In certain embodiments, the costimulatory domain comprises the amino acid sequence of SEQ ID NO: 20.

In certain embodiments, the costimulatory domain comprises a conservative substitution relative to a costimulatory domain disclosed herein, e.g., relative to costimulatory domain comprising the amino acid sequence of SEQ ID NO: 20. In certain embodiments, the costimulatory domain comprises less than 2, less than 3, less than 4, less than 5, less than 6, less than 7, less than 8, less than 9, less than 10, less than 11, less than 12, less than 13, less than 14, or less than 15 conservative substitutions relative to, e.g., the amino acid sequence of SEQ ID NO: 20. In certain embodiments, the intracellular signaling domain comprises 1, 2, 3, 4, 5, 6, 7, 8, 9, 10, 11, 12, 13, 14, or 15 or more conservative substitutions relative to the amino acid sequence of SEQ ID NO: 20.

In certain embodiments, a CAR of the disclosure comprises a costimulatory domain comprising a CD28 intracellular domain. In certain embodiments, the costimulatory domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 62. In certain embodiments, the costimulatory domain comprises the amino acid sequence of SEQ ID NO: 62.

In certain embodiments, the costimulatory domain comprises a conservative substitution relative to a costimulatory domain disclosed herein, e.g., relative to costimulatory domain comprising the amino acid sequence of SEQ ID NO: 62. In certain embodiments, the costimulatory domain comprises less than 2, less than 3, less than 4, less than 5, less than 6, less than 7, less than 8, less than 9, less than 10, less than 11, less than 12, less than 13, less than 14, or less than 15 conservative substitutions relative to, e.g., the amino acid sequence of SEQ ID NO: 62. In certain embodiments, the intracellular signaling domain comprises 1, 2, 3, 4, 5, 6, 7, 8, 9, 10, 11, 12, 13, 14, or 15 or more conservative substitutions relative to the amino acid sequence of SEQ ID NO: 62.

The intracellular signaling and costimulatory domains within the cytoplasmic portion of the CAR may be linked to each other in a random or specified order. In certain embodiments, a costimulatory signaling domain is deployed N-terminal to the primary signaling domain. Optionally, the intracellular domains are linked by a short oligo- or polypeptide linker, for example, between 2 and 10 amino acids in length. In certain embodiments, the intracellular domains are linked by, e.g., a GS doublet or by a (G₄S)ₙ linker. In certain embodiments, the intracellular signaling domain (e.g. CD3ζ signaling domain) is the C-terminal domain of the CAR.

d. Other CAR Domains

The extracellular antigen-binding domain of the CAR can be connected to the transmembrane domain by a hinge domain or linker. In certain embodiments, the hinge domain or linker is interposed between the antigen binding site and the transmembrane domain. A variety of hinges or linkers can be employed, including, but not limited to, the human Ig (immunoglobulin) hinge (e.g., an IgG4 hinge, an IgD hinge), a Gly-Ser linker, a (G₄S)₄ linker, a KIR2DS2 hinge, and a CD8α hinge.

In certain embodiments, the hinge domain is a CD8α hinge. In certain embodiments, the hinge domain comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 22. In certain embodiments, the hinge domain comprises the amino acid sequence of SEQ ID NO: 22.

In certain embodiments, the hinge domain comprises a conservative substitution relative to a hinge domain disclosed herein, e.g., relative to a hinge domain comprising the amino acid sequence of SEQ ID NO: 22. In certain embodiments, the hinge domain comprises less than 2, less than 3, less than 4, less than 5, less than 6, less than 7, less than 8, less than 9, less than 10, less than 11, less than 12, less than 13, less than 14, or less than 15 conservative substitutions relative to, e.g., the amino acid sequence of SEQ ID NO: 22. In certain embodiments, the hinge domain comprises 1, 2, 3, 4, 5, 6, 7, 8, 9, 10, 11, 12, 13, 14, or 15 or more conservative substitutions relative to the amino acid sequence of SEQ ID NO: 22.

In certain embodiments, the CAR comprises a CD8α transmembrane domain and a CD8α hinge.

In certain embodiments, the CAR comprises a signal peptide. In certain embodiments, the nucleic acid encoding the CAR comprises a nucleic acid sequence encoding a signal peptide. In certain embodiments, the signal peptide is derived from a native polypeptide. In other embodiments, the signal peptide comprises a heterologous or non-native signal peptide. In certain embodiments, the signal peptide is a CD8α signal peptide or an IgG signal peptide.

In certain embodiments, the CAR comprises a CD8α signal peptide. In certain embodiments, the signal peptide comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 25. In certain embodiments, the signal peptide comprises the amino acid sequence of SEQ ID NO: 25. In certain embodiments, the CAR comprises an IgG signal peptide. In certain embodiments, the signal peptide comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 26. In certain embodiments, the signal peptide comprises the amino acid sequence of SEQ ID NO: 26.

In certain embodiments, the signal peptide comprises a conservative substitution relative to a signal peptide disclosed herein, e.g., relative to a signal peptide comprising the amino acid sequence of SEQ ID NO: 25 or SEQ ID NO: 26. In certain embodiments, the signal peptide comprises less than 2, less than 3, less than 4, less than 5, less than 6, less than 7, less than 8, less than 9, or less than 10 conservative substitutions relative to, e.g., the signal peptide of SEQ ID NO: 25 or SEQ ID NO: 26. In certain embodiments, the signal peptide comprises 1, 2, 3, 4, 5, 6, 7, 8, 9, or 10 or more conservative substitutions relative to the amino acid sequence of SEQ ID NO: 25 or SEQ ID NO: 26.

e. Exemplary CAR Constructs

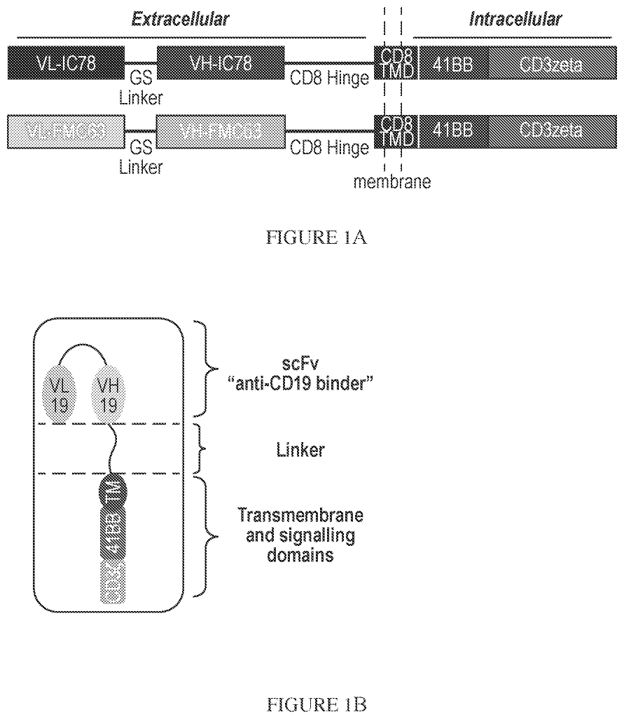

In certain embodiments, a CAR of the disclosure comprises, in the N- to C-terminal direction: (1) an extracellular domain comprising an antigen binding site, for example, an scFv such as an anti-CD19 scFv, e.g., an scFv comprising the amino acid sequence of SEQ ID NO: 9; (2) an optional hinge domain or linker, for example, a CD8α hinge, e.g., a hinge comprising the amino acid sequence of SEQ ID NO: 22; (3) a transmembrane domain, for example, a CD8α transmembrane domain, e.g., a transmembrane domain comprising the amino acid sequence of SEQ ID NO: 19; (4) an optional costimulatory domain, for example, an intracellular domain of 4-1BB, e.g., a costimulatory domain comprising the amino acid sequence of SEQ ID NO: 20; and (e) an intracellular signaling domain, for example, a CD3ζ signaling domain, e.g., a signaling domain comprising the amino acid sequence of SEQ ID NO: 21. In certain embodiments, the CAR further comprises an N-terminal signal peptide, for example, a CD8 signal peptide, e.g., a signal peptide comprising the amino acid sequence of SEQ ID NO: 25. Certain exemplary anti-CD19 CAR constructs of the disclosure are depicted in FIG. 1.

In certain embodiments, a CAR of the disclosure comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 23. In certain embodiments, the CAR comprises the amino acid sequence of SEQ ID NO: 23. In certain embodiments, a CAR of the disclosure comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 27. In certain embodiments, the CAR comprises the amino acid sequence of SEQ ID NO: 27. In certain embodiments, a CAR of the disclosure comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 63. In certain embodiments, the CAR comprises the amino acid sequence of SEQ ID NO: 63. In certain embodiments, a CAR of the disclosure comprises an amino acid sequence at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the amino acid sequence of SEQ ID NO: 64. In certain embodiments, the CAR comprises the amino acid sequence of SEQ ID NO: 64.

In certain embodiments, a CAR of the disclosure comprises a conservative substitution relative to a CAR disclosed herein, e.g., relative to a CAR comprising the amino acid sequence of SEQ ID NO: 23, SEQ ID NO: 27, SEQ ID NO: 63, or SEQ ID NO: 64. In certain embodiments, the CAR comprises less than 2, less than 3, less than 4, less than 5, less than 6, less than 7, less than 8, less than 9, less than 10, less than 11, less than 12, less than 13, less than 14, less than 15, less than 20, less than 25, or less than 30 conservative substitutions relative to, e.g., the CAR of SEQ ID NO: 23, SEQ ID NO: 27, SEQ ID NO: 63, or SEQ ID NO: 64. In certain embodiments, the CAR comprises 1, 2, 3, 4, 5, 6, 7, 8, 9, 10, 11, 12, 13, 14, 15, 20, 25, or 30 or more conservative substitutions relative to the amino acid sequence of SEQ ID NO: 23, SEQ ID NO: 27, SEQ ID NO: 63, or SEQ ID NO: 64.

In some embodiments the CAR is encoded by a nucleic acid sequence at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the nucleic acid sequence of SEQ ID NO: 28 or SEQ ID NO: 29. In some embodiments the CAR is encoded by a nucleic acid sequence comprising the nucleic acid sequence of SEQ ID NO: 28 or SEQ ID NO: 29.

f. Vectors Comprising CAR-Encoding Polynucleotides

In certain embodiments, CARs for use in the methods of the disclosure are encoded by polynucleotides which, optionally, may be present in a vector. In certain embodiments, the polynucleotide encodes a CAR comprising an extracellular domain comprising an antigen binding site, a transmembrane domain, an intracellular signaling domain and, optionally, a costimulatory domain. In certain embodiments, the polynucleotide encodes a CAR comprising an anti-CD19 scFv, a CD8α transmembrane domain, a 4-1BB costimulatory domain, and a CD3ζ signaling domain.

In certain embodiments, the vector comprises a plasmid vector, viral vector, retroviral vector, lentiviral vector, adenoviral vector, AAV, retrotransposon (e.g., piggyback, sleeping beauty), site directed insertion vector (e.g., CRISPR, Zinc finger nucleases, TALEN), or suicide expression vector, or other known vector in the art.

In certain embodiments, the vector is a viral vector, e.g., a lentiviral vector, e.g., a 3rd generation lentiviral vector, e.g., a 3^{rd} generation self-inactivating lentiviral vector.

In certain embodiments, the vector is an RNA vector, e.g., an mRNA vector. This can achieve the same therapeutic effect as in a virally transduced cell, but would not be permanent because the mRNA would dilute out with cell division. Depending upon the circumstances, the RNA vector can be electroporated into the host cell or introduced into the host cell by a lipid nanoparticle (LNP), e.g., a targeted LNP.

Expression of the CAR can be verified by sequencing. Expression of the full length CAR protein may be verified using immunoblot, immunohistochemistry, flow cytometry, or other technology well known and available in the art.

The present disclosure also provides a vector in which DNA or RNA encoding the CAR of the present disclosure is inserted. Vectors, including those derived from retroviruses such as lentivirus, are suitable tools to achieve long-term gene transfer since they allow long-term, stable integration of a transgene and its propagation in daughter cells. Lentiviral vectors have the added advantage over vectors derived from onco-retroviruses, such as murine leukemia viruses, in that they can transduce non-proliferating cells, such as hepatocytes. They also have the added advantage of resulting in low immunogenicity in the subject into which they are introduced.

In brief summary, the expression of natural or synthetic polynucleotides encoding CARs is typically achieved by operably linking a nucleic acid encoding the CAR polypeptide or portions thereof to a promoter, and incorporating the construct into an expression vector. The vector is one generally capable of replication in a mammalian cell, and/or also capable of integration into the cellular genome of the mammal. Typical vectors contain transcription and translation terminators, initiation sequences, and promoters useful for regulation of the expression of the desired nucleic acid sequence.

The nucleic acid can be cloned into any number of different types of vectors. For example, the nucleic acid can be cloned into a vector including, but not limited to, a plasmid, a phagemid, a phage derivative, an animal virus, and a cosmid. Vectors of particular interest include expression vectors, replication vectors, probe generation vectors, and sequencing vectors.

The expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 2001/96584; WO 2001/29058; and U.S. Pat. No. 6,326,193).

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. Depending on the promoter, it appears that individual elements can function either cooperatively or independently to activate transcription.

An example of a promoter that may be used is the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, the elongation factor-1α promoter (EF-1α), as well as human gene promoters such as, but not limited to, the actin promoter, the myosin promoter, or the hemoglobin promoter, as appropriate.

Further, the disclosure should not be limited to the use of constitutive promoters. Inducible promoters are also contemplated for use in the methods and compositions of the disclosure. The use of an inducible promoter provides a molecular switch capable of turning on expression of the polynucleotide sequence, which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter. In some embodiments, an inducible promoter is activated in response to an extracellular ligand. For example, in some embodiments, the inducible promoter is activated (and the expression of the CAR is regulated) by an extracellular ligand binding to a synthetic receptor. For example, in some embodiments, a synthetic receptor, e.g., a synthetic Notch receptor (i.e., “synNotch”) may be employed as a binding-triggered transcriptional switch that, when bound to its ligand, activates a promoter to which a nucleic acid sequence encoding the CAR is operably linked. Accordingly, as a non-limiting example, such systems may require the presence of a ligand (e.g., to which the synNotch binds) for the immune cell to be responsive to a BCR or autoantibody (e.g., to which the CAR binds). The requirement of particular combinations to generate certain signaling outputs in molecular circuits results in a logic gate. See, for example, Roybal et al., 2016 CELL 164(4):770-9.

Examples of other systems for expressing or regulating expression of a chimeric receptor include those described in Wu et al. (2015) Science 350: aab4077; Fedorov et al. (2014) Cancer Journal 20:160-165; Kloss et al. (2013) Nature Biotechnology 31: 71-75; Sakemura et al. (2016) Cancer Immunol. Res. 4:658-668; Hill et al. (2018) NAT. CHEM. BIOL. 14: 112-117; Di Stasi et al. (2011) N. ENGL. J. MED. 365: 1673-1683; Budde et al. (2013) PLOS ONE 8: e82742; Wei et al. (2012) NATURE 488: 384-388; Ma et al. (2016) PROC. NATL. ACAD. SCI. USA 113: E450-458; Rodgers et al. (2016) PROC. NATL. ACAD. SCI. USA 113: E459-468; Kudo et al. (2014) CANCER RES. 74: 93-103, and Chen et al. (2010) PROC. NATL. ACAD. SCI. USA 107: 8531-8536.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. The selectable marker may be carried on a separate piece of DNA or RNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA or RNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS LETTERS 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY).

Biological methods for introducing a polynucleotide of interest into a host cell include the use of DNA and RNA vectors. RNA vectors include vectors having an RNA promoter and/other relevant domains for production of an RNA transcript. Viral vectors, and especially retroviral vectors, have become the most widely used method for inserting genes into mammalian, e.g., human cells. Other viral vectors may be derived from lentivirus, poxviruses, herpes simplex virus, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of targeted delivery of nucleic acids are known, such as delivery of polynucleotides with targeted nanoparticles or another suitable sub-micron sized delivery system.

In the case where a non-viral delivery system is utilized, an exemplary delivery vehicle is a liposome or lipid nanoparticle (LNP). The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA, lipid/RNA, or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances, which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Examples of lipids, liposomes, lipid nanoparticles, and related formulations are described, for example, in U.S. Pat. Nos. 8,058,069, 8,492,359, 8,822,668, 9,006,417, 9,139,554, 9,364,435, 9,404,127, 9,415,109, 9,504,651, 9,518,272, 9,567,296, 9,580,711, 9,636,414, 9,694,077, 9,758,795, 9,814,777, 9,868,692, 9,878,042, 9,943,846, 9,950,065, 10,041,091, 10,106,490, 10,166,298, 10,221,127, 10,227,302, 10,233,148, 10,266,485, 10,442,756, 10,485,884, 10,561,732, 10,576,146, 10,577,403, 10,653,780, 10,703,789, 10,980,895, 11,045,418, 11,141,378, 11,173,120, 11,191,849, 11,285,222, 11,357,856, 11,446,383, 11,453,639, 11,478,552, 11,559,587, U.S. Patent Application Publication Nos. 2011/0117125, 2012/0264810, 2018/0000953, 2018/0085474, 2018/0185516, 2019/0022247, 2019/0032087, 2019/0274968, 2019/0336608, 2020/0046830, 2020/0109113, 2020/0155671, 2020/0163878, 2020/0164038, 2020/0172472, 2020/0297634, 2020/0297870, 2020/0306191, 2021/0145982, 2021/0207140, 2021/0220274, 2021/0346306, 2022/0000778, 2022/0001029, 2022/0118112, and 2022/0160899; and International Patent Application Publication Nos. 2008/042973, 2021/231929, and 2021/237084.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In certain embodiments wherein a non-viral delivery method is utilized, an exemplary delivery vehicle is a bioabsorbable silicon nanoparticle. Silicon nanoparticles can be made of either pure silicon or a hydrolysable silicon-containing material, and can be made porous by standard techniques such as contacting the particles with a hydrofluoric acid (HF)/ethanol mixture and applying a current. Silicon nanoparticles may be loaded with a polynucleotide, e.g., RNA, to be delivered into a host cell (e.g., in vitro, ex vivo, or in vivo). Silicon nanoparticles may be surface-treated with a lipid (e.g., phosphatidylcholine (PC), phosphatidylethanolamine (PE), stearylamine (SA), and/or lecithin, which can aid in controlling the rate of release of the payload polynucleotide. A lipid-surface-treated silicon nanoparticle may be further treated with an amino acid (e.g., arginine, histidine, and/or glycine), which can promote stability of the payload nucleic acid, e.g., RNA. Examples of silicon nanoparticles are described, for example, in U.S. Pat. No. 9,132,083, in U.S. Patent Application Publication Nos. 2022/0183989 and 2022/0184038, and in International Patent Application Publication No. 2020/193999.

Any domains and/or fragments of the CAR, vector, and the promoter may be synthesized gene fragments amplified by PCR or any other means known in the art.

### IV. Pharmaceutical Compositions

For therapeutic use, an agent (e.g., cyclophosphamide, fludarabine, or a cell disclosed herein) preferably is present in a pharmaceutical composition, optionally wherein the composition comprises a pharmaceutically acceptable carrier.

a. Compositions Comprising Cyclophosphamide and/or Fludarabine

In certain embodiments, cyclophosphamide and/or fludarabine are preferably present in a pharmaceutical composition. Cyclophosphamide and fludarabine may be present in the same or different compositions, as appropriate.

A pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, e.g., Adeboye Adejare, Remington: The Science and Practice of Pharmacy (23d ed. 2020)).

In certain embodiments, a pharmaceutical composition may contain nanoparticles, e.g., polymeric nanoparticles, liposomes, or micelles (see, Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, an agent (e.g., cyclophosphamide or fludarabine) disclosed herein is administered by IV infusion. Useful formulations can be prepared by methods known in the pharmaceutical art. For example, see Adeboye Adejare, Remington: The Science and Practice of Pharmacy (23d ed. 2020). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor ELTM (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

An intravenous drug delivery formulation may be contained in a syringe, pen, or bag. In certain embodiments, the bag may be connected to a channel comprising a tube and/or a needle. In certain embodiments, the formulation may be a lyophilized formulation or a liquid formulation. In certain embodiments, the formulation may be freeze-dried (lyophilized) and contained in one or more vials. In certain embodiments, freeze dried formulation from the one or more vials are combined to obtain a therapeutic dose of the agent.

In certain embodiments, a pharmaceutical composition may contain a stabilizing agent. In certain embodiments, the stabilizing agent is a cation, such as a divalent cation. In certain embodiments, the cation is calcium or magnesium. The cation can be in the form of a salt, such as calcium chloride (CaCl₂)) or magnesium chloride (MgCl₂). In certain embodiments, the stabilizing agent is present in an amount from about 0.05 mM to about 5 mM.

In certain embodiments, pharmaceutical formulations are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. In a preferred embodiment, the pharmaceutical composition is administered subcutaneously and, in an even more preferred embodiment, intravenously. Preparations for parenteral administration include sterile aqueous or non-aqueous solutions, suspensions, and emulsions.

The therapeutically effective amount of the agent to be administered will depend on variables such as the type and extent of disease or indication to be treated, the overall health of the patient, the in vivo potency of the agent, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Dosing frequency can vary, depending on factors such as route of administration, dosage amount, and the disease being treated. A preferred route of administration is intravenous infusion.

b. Immune Cell Therapy Compositions

As described hereinabove, the engineered immune cells of the disclosure are useful in immune cell therapies (e.g., adoptive immune cell therapies, e.g., for the treatment of an autoimmune disease). Accordingly, the present disclosure provides compositions comprising one or more engineered immune cells as described herein. In certain embodiments, the one or more engineered immune cells are present in a pharmaceutical composition, e.g., wherein the composition comprises a pharmaceutically acceptable carrier.

In certain embodiments, the immune cell therapy is autologous, i.e., immune cells obtained from a patient, after in vitro culture, are administered to the same patient. In certain embodiments, the immune cell therapy is allogeneic and the immune cells are obtained from a healthy donor, optionally wherein the immune cells (e.g., T cells) are genetically engineered to inactivate or lack expression of a functional T cell receptor (TCR) and/or a human leukocyte antigen (HLA) molecule, e.g., an HLA class I component or an HLA class II component. For example, in certain embodiments, an allogeneic T cell can be engineered to have reduced or no expression of a functional TCR on its surface, or engineered to have reduced or no expression of one or more subunits that comprise a functional TCR (e.g., the TCR α chain or the TCR β chain). Alternatively, the T cell can express a functionally impaired TCR, e.g., by expression of mutated or truncated forms of one or more of the subunits of the TCR. In certain embodiments, an allogeneic T cell can be engineered to have reduced or no expression of a functional HLA molecule on its surface, e.g., reduced or no expression of an HLA class I molecule or an HLA class II molecule. In certain embodiments, surface expression of an HLA class I molecule is reduced in an allogeneic T cell by targeting or knocking out a sequence encoding beta-2 microglobulin (β2M). In certain embodiments, surface expression of an HLA class II molecule is reduced in an allogeneic T cell by targeting or knocking out a sequence encoding CIITA. Such cells can be created through the use of a gene editing systems as described herein. In embodiments, gene editing systems targeting sequences encoding TCR α chain, TCR β chain, β2M, and/or CIITA are introduced into the cells, such that surface expression of functional TCR, HLA class I molecules, and/or HLA class II molecules is downregulated. In some embodiments, the allogeneic T cell can lack a functional TCR and a functional HLA molecule, e.g., an HLA class I molecule and/or an HLA class II molecule.

An immune cell therapy can be provided as a cell composition. In certain embodiments, the engineered immune cell in the composition is an NK cell, e.g., a CAR NK cell. In certain embodiments, the engineered immune cell in the composition is a T cell, e.g., a CAR T cell. It is understood that other types of cells, such as APCs, may be used for expanding T cells ex vivo. As such, the cell composition may include other cell types in addition to T cells. In certain embodiments, the cell composition has been enriched for T cells. Where the T cells are prepared by stimulation using APCs in an ex vivo cell culture, the T cells can be enriched by methods known in the art. For example, in certain embodiments, the APCs are removed from the cell culture by surface marker-based magnetic bead selection or cell sorting. In certain embodiments, the APCs are outgrown by T cells under conditions (e.g., cytokines) that preferably support T cell proliferation. In certain embodiments, the APCs are removed by their stronger adherence to tissue culture plate than T cells. The enrichment can produce a composition in which at least 70%, at least 80%, at least 90%, at least 95%, or at least 99% of the cells in the composition are T cells. In certain embodiments, the composition is substantially free of myeloid cells. For example, in certain embodiments, the percentage of myeloid cells relative to all cells in the composition is 20% or less, 10% or less, 5% or less, 4% or less, 3% or less, 2% or less, or 1% or less.

In certain embodiments, an immune cell composition may comprise one or more immunogenicity enhancing adjuvants (also referred to as “adjuvants” herein). Such adjuvants are substances that enhance or potentiate the immune response (e.g., immune responses mediated by CD8-positive T cells and helper-T (TH) cells to an antigen) in a non-antigen-specific manner, and would thus be considered useful in a pharmaceutical composition disclosed herein. Suitable adjuvants include, but are not limited to, 1018 ISS, aluminum salts, AMPLIVAX®, AS15, BCG, CP-870,893, CpG7909, CyaA, dSLIM, flagellin or TLR5 ligands derived from flagellin, FLT3 ligand, GM-CSF, IC30, IC31, lmiquimod (ALDARA®), resiquimod, IMUFACT®, IMP321, interleukins as IL-2, IL-13, IL-21, interferon-α or -β, or pegylated derivatives thereof, IS Patch, ISS, ISCOMATRIX, ISCOMs, JUVIMMUNE®, LIPOVAC®, MALP2, MF59, monophosphoryl lipid A, Montanide IMS 1312, Montanide ISA 206, Montanide ISA 50V, Montanide ISA-51, water-in-oil and oil-in-water emulsions, OK-432, OM-174, OM-197-MP-EC, ONTAK®, OspA, poly(lactide coglycolide) [PLG]-based and dextran microparticles, talactoferrin SRL 172, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, QUIL®, or Superfos. Depending upon the circumstances, adjuvants such as Freund's or GM-CSF may be preferred. Several immunological adjuvants (e.g., MF59) specific for dendritic cells and their preparation have been described previously (Ott et al. (1995) PHARM BIOTECHNOL. 6: 277-96). In certain embodiments, an adjuvant is a naturally occurring adjuvant. In certain embodiments, an adjuvant is a non-naturally occurring adjuvant.

The immune cell therapy compositions can further comprise one or more carriers and/or excipients. Exemplary carriers and excipients are described herein (see the “Compositions Comprising Cyclophosphamide and/or Fludarabine” subsection above).

The therapeutically effective amount of the engineered immune cells to be administered may depend on variables such as the type and extent of disease or indication to be treated, the overall health of the patient, the in vivo potency of the agent, the pharmaceutical formulation, and the route of administration. A preferred route of administration is intravenous infusion.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps. Similarly, throughout the description, where compositions are described as consisting essentially of specific components, or where processes and methods are described as consisting essentially of specific steps, it is contemplated that, additionally, there are compositions of the present invention that consist of the recited components, and that there are processes and methods according to the present invention that consist of the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

It should be understood that the order of steps or order for performing certain actions is immaterial so long as the method remains operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This example describes the treatment of systemic lupus erythematosus in a patient by administering T cells engineered to express a CD19-specific CAR, after administration of a preconditioning regimen comprising reduced doses of cyclophosphamide and fludarabine.

A patient having systemic lupus erythematosus was treated using an autologous CD19-specific CAR T cell therapy using essentially the same cell therapeutic methods as described in Mackensen et al. (2022), except that the patient was administered a lymphodepleting preconditioning regimen comprising a reduced dose of cyclophosphamide and a reduced dose of fludarabine. Briefly, autologous T cells were obtained from the patient and transduced with a lentiviral anti-CD19 CAR vector, which encoded an anti-CD19-CAR construct comprising an scFv which specifically binds CD19, a CD8α transmembrane domain, a 4-1BB costimulatory domain, and a CD3ζ signaling domain. The anti-CD19 scFv comprised the amino acid sequence of SEQ ID NO: 18. The transduced T cells were expanded. The patient was administered a reduced-dose preconditioning regimen prior to CAR T cell infusion: a dose of 12.5 mg/m²/day fludarabine was administered intravenously on Days −5, −4, and −3, and a dose of 500 mg/m²/day cyclophosphamide was administered intravenously on Day −3. A dose of 1×10⁶ CAR T cells per kg bodyweight was administered to the patient as a single intravenous infusion on Day 0. The patient was monitored post-infusion for SLE disease markers, B-cell levels, white blood cell count, and CAR T cell levels.

The CAR T cell population successfully expanded in the subject post-infusion, in spite of the reduced preconditioning regimen. At 6 weeks post-infusion, CAR T cell engraftment, B cell aplasia, and clinical impact were similar in the patient that received the reduced preconditioning dose as compared to the five patients described in Mackensen et al. (2022) who received the full-dose preconditioning regimen (25 mg/m²/day fludarabine on Days −5, −4, and 3, and 1,000 mg/m²/day cyclophosphamide on Day −3). The patient that received the reduced preconditioning regimen also exhibited a lesser drop in white blood cell count, as compared to the patients who received the full-dose preconditioning regimen.

As was observed in the five CD19-CAR-treated patients who received a full dose of the preconditioning regimen, the SLE patient who received a reduced preconditioning regimen went into remission. As of 5 months post-infusion, the patient remained in remission and required no further treatment. No treatment-related adverse events were reported for the patient receiving a reduced-dose preconditioning regimen.

Collectively, these data demonstrate that the use of a full-dose preconditioning regimen comprising a total dose of 1,000 mg/m² cyclophosphamide and a total dose of 75 mg/m² fludarabine may not be necessary for treatment of SLE with CD19-CAR T cell therapy, and that the use of a reduced-dose preconditioning regimen (a total dose of 500 mg/m² cyclophosphamide and 37.5 mg/m²) may also be effective for treatment of SLE with CD19-CAR T cell therapy.

### Example 2

This example describes the treatment of myositis in a patient by administering T cells engineered to express a CD19-specific CAR, after administration of a preconditioning regimen comprising administering reduced doses of cyclophosphamide and fludarabine.

A patient having dermatomyositis was treated using an autologous CD19-specific CAR T cell therapy using essentially the same cell therapeutic methods as described in Müller et al. (Müller et al. (2023) “CD19-targeted CAR T cells in refractory antisynthetase syndrome,” LANCET), except that the patient was administered a lymphodepleting preconditioning regimen comprising a reduced dose of cyclophosphamide and a reduced dose of fludarabine. Briefly, autologous T cells were obtained from the patient and transduced with a lentiviral anti-CD19 CAR vector, which encoded an anti-CD19-CAR construct comprising an scFv which specifically binds CD19, a CD8α transmembrane domain, a 4-1BB costimulatory domain, and a CD3ζ signaling domain. The anti-CD19 scFv comprised the amino acid sequence of SEQ ID NO: 18. The transduced T cells were expanded. The patient was administered a reduced-dose preconditioning regimen prior to CAR T cell infusion: a dose of 12.5 mg/m²/day fludarabine was administered intravenously on Days −5, −4, and −3, and a dose of 500 mg/m²/day cyclophosphamide was administered intravenously on Day −3. A dose of 1×10⁶ CAR T cells per kg bodyweight was administered to the patient as a single intravenous infusion on Day 0. The patient was monitored post-infusion for myositis disease markers.

As was observed in the anti-synthetase syndrome myositis patient treated with CD19-CAR T cell therapy using a full-dose preconditioning regimen reported on in Müller et al. (2023), the dermatomyositis patient who received a reduced preconditioning regimen exhibited a similar response, with comparable peak CD19 CAR T expansion post infusion and a complete resolution of disease which continued out to at least 5 months. No treatment-related adverse events were reported for the patient receiving a reduced preconditioning regimen. A higher nadir of WBC post preconditioning was also observed, although statistical analysis cannot be performed.

This example demonstrates that autologous CD19-CAR T cell therapy, in combination with a lymphodepleting preconditioning regimen, can be an effective treatment for myositis. Additionally, this Example demonstrates that the use of a preconditioning regimen comprising a total dose of 1,000 mg/m² cyclophosphamide and a total dose of 75 mg/m² fludarabine may not be necessary for treatment of myositis with CD19-CAR T cell therapy, and that the use of a reduced-dose preconditioning regimen (a total dose of 500 mg/m² cyclophosphamide and 37.5 mg/m²) may also be effective.

### Example 3

This example describes a phase I/II open-label, single-arm and randomized parallel-group study to investigate the safety and efficacy of autologous CD19-specific CAR T cells (CAB-001) in subjects with active systemic lupus erythematosus. Treatment efficacy is analyzed using either a standard full-dose preconditioning regimen (a total dose 1,000 mg/m² cyclophosphamide and a total dose of 75 mg/m² fludarabine) or a reduced-dose preconditioning regimen.

### Background

SLE is a chronic autoimmune disorder characterized by autoantibody production and abnormal B cell function. SLE presents with fluctuating severity and may cause tissue damage in a variety of organs over time. Lupus nephritis (LN) is a common severe manifestation of SLE, which can lead to significant morbidity and mortality. There is no approved cure for SLE, and available treatment options have limited effectiveness in SLE patients with serious long-term side effects.

### Investigational Product

The investigational product, CAB-001, is an autologous CD19-targeting chimeric antigen receptor (CAR) T cell. The CAR comprises a fully human scFv which specifically binds CD19, a CD8α transmembrane domain, a 4-1BB costimulatory domain, and a CD3ζ signaling domain. The anti-CD19 scFv comprises the amino acid sequence of SEQ ID NO: 9, and the construct comprises the amino acid sequence of SEQ ID NO: 23.

### Objectives and Endpoints

A primary objective of this study is to evaluate the safety and tolerability of CAB-001 in subjects with active SLE over 28 days, measured by incidence of adverse events (AEs) occurring within 28 days after CAB-001 infusion, including dose-limiting toxicities (DLTs) and AEs that are related to CAB-001.

A secondary objective of this study is to evaluate the safety and tolerability of CAB-001 in subjects with active SLE over 156 weeks, measured by AEs, vital signs, physical examination, and clinical laboratory tests occurring within 156 weeks after CAB-001 infusion.

An additional secondary objective of this study is to evaluate the effect of CAB-001 regimen on white blood cell counts, including B cell counts, measured from baseline in white blood cell counts with differential, including B cell counts.

An additional secondary objective of this study is to evaluate CAB-001 expansion and persistence in vivo, measured by the approximate number of CAB-001 cells and CAB-001 markings per cell number in subjects.

An additional secondary objective of this study is to evaluate the effect of CAB-001 on SLE serology, measured by the changes from baseline in anti-double stranded DNA (dsDNA) antibodies, complement component 3 (C3), complement component 4 (C4), and 50% hemolytic complement (CH50).

An additional secondary objective of this study is to evaluate the effect of CAB-001 on SLE disease activity, measured as: the changes in urine protein:creatinine ratio (UPCR), estimated glomerular filtration rate (eGFR), SLEDAI-2K score, British Isles Lupus Assessment Group (BILAG)-2004 score, Physician Global Assessment (PGA) score, joint counts, Cutaneous Lupus Erythematosus Disease Area and Severity Index (CLASI) score, and the SLICC Damage Index (SDI); and proportions of subjects achieving complete renal response (CRR), SRI-4, SRI-5, SRI-6, BICLA responses, LLDAS and DORIS remission criteria.

An additional secondary objective of this study is to evaluate the effect of CAB-001 on concomitant corticosteroid use and other SLE-related therapy, measured by the change from baseline in the dose of concomitant corticosteroids and other SLE-related therapies, proportion of subjects achieving a dose ≤5 mg/day of oral prednisone (or equivalent), and proportion of subjects who require no SLE-related therapies.

An additional secondary objective of this study is to evaluate the effect of CAB-001 on patient-reported outcomes and health-related quality of life, measured as changes from baseline in 36-Item Short Form Survey Version 2 (SF-36v2), Pain Numeric Rating Scale (NRS), Functional Assessment of Chronic Illness Therapy-Fatigue (FACIT-F), Patient Global Assessment (PtGA), Lupus Quality of Life Scale (Lupus QoL), and EQ-5D-5L scores.

An additional secondary objective of this study is to evaluate the effect of CAB-001 on disease flares, measured as the incidences of mild/moderate and severe disease flares, as defined per BILAG-2004 and modified SELENA-SLEDAI Flare Index (SFI), and as the time to mild/moderate and severe flare.

Additional objectives and endpoints relate to a sub-study, detailed in “Part B” below, which is conducted to further evaluate the efficacy of CAB-001 in the treatment of lupus nephritis (LN).

A primary objective of this sub-study is to compare the efficacy of CAB-001 and belimumab plus standard of care at Week 52 in subjects with LN in complete renal response, measured as the proportion of subjects achieving complete renal response at Week 52.

A secondary objective of this sub-study is to compare the efficacy of CAB-001 and belimumab plus standard of care in complete renal response, measured as: the proportion of subjects achieving complete renal response at Week 24, the proportion of subjects achieving complete renal response and on no other SLE-related therapy at Weeks 24 and 52, the time to the first achievement of complete renal response, and the time to the first achievement of complete renal response and on no other SLE-related therapy.

An additional secondary objective of this sub-study is to compare the efficacy of CAB-001 and belimumab plus standard of care in UPCR response, measured as the proportion of subjects achieving UPCR≤0.5 mg/mg at Weeks 24 and 52, time to the first achievement of UPCR≤0.5 mg/mg, and change from baseline in UPCR at weeks 24 and 52.

An additional secondary objective of this sub-study is to compare the efficacy of CAB-001 and belimumab plus standard of care in renal function, measured as the change from baseline in estimated glomerular filtration rate (eGFR) at weeks 24 and 52.

An additional secondary objective of this sub-study is to compare the efficacy of CAB-001 and belimumab plus standard of care in SRI-4 response, measured as the proportion of subjects achieving SRI-4 response at Week 24 and Week 52.

An additional secondary objective of this sub-study is to compare the efficacy of CAB-001 and belimumab plus standard of care in BICLA response, measured as the proportion of subjects achieving BICLA response at Week 24 and Week 52.

An additional secondary objective of this sub-study is to compare the efficacy of CAB-001 and belimumab plus standard of care in SLEDAI-2K response, measured as the change from baseline SLEDAI-2K score at timepoints through Week 52.

An additional secondary objective of this sub-study is to compare the efficacy of CAB-001 and belimumab plus standard of care in the achievement of low disease, measured as the proportion of subjects achieving Lupus Low Disease Activity State (LLDAS) at Week 24 and Week 52.

An additional secondary objective of this sub-study is to compare the efficacy of CAB-001 and belimumab plus standard of care in the achievement of remission, measured as the proportion of subjects achieving Definition of Remission in SLE (DORIS) at Weeks 24 and 52.

An additional secondary objective of this sub-study is to compare the effect of CAB-001 and belimumab plus standard of care in B-cell count, measured as the change in peripheral B-cell count over time through Week 52.

An additional secondary objective of this sub-study is to compare the effect of CAB-001 and belimumab plus standard of care in SLE serology, measured as the changes from baseline in anti-dsDNA antibody levels through Week 52, and the changes from baseline in C3, C4, and CH50 levels through Week 52

An additional secondary objective of this sub-study is to compare the efficacy of CAB-001 and belimumab plus standard of care in corticosteroid use, measured as the change from baseline in the dose of concomitant corticosteroids at Weeks 24 and 52, and as the proportion of subjects achieving a dose of ≤5 mg/day oral prednisone or equivalent at Weeks 24 and 52.

An additional secondary objective of this sub-study is to compare the efficacy of CAB-001 and belimumab plus standard of care in disease flare, measured as the incidence of mild/moderate and severe disease flare per BILAG-2000 and SFI through Week 52, and as time to mild/moderate and severe disease flare.

An additional secondary objective of this sub-study is to compare the effect of CAB-001 and belimumab plus standard of care in patient-reported outcomes and health-related QOL, measured as the change from baseline in SF-36v2 Health Survey, Pain Numeric Rating Scale, FACIT-F, PtGA, Lupus QoL, and EQ-5D-5L scores at Weeks 24 and 52.

An additional secondary objective of this sub-study is to evaluate the effect of CAB-001 on disease activity and response at timepoints from Week 56 to Week 156 post-infusion, measured as: change from baseline in UPCR, eGFR, SLEDAI-2K, BILAG-2004, PGA, joint counts, CLASI, and SDI; proportion of subjects achieving complete renal response; proportion of subjects achieving complete renal response on no SLE-related therapy; proportion of subjects achieving UPCR≤0.5 mg/mg; and as the proportion of subjects achieving SRI-4, SRI-5, SRI-6, and BICLA responses and LLDAS and DORIS criteria.

An additional secondary objective of this sub-study is to evaluate the effect of CAB-001 on concomitant steroid use and SLE-related therapy at time points from Week 56 to Week 156 post-infusion, measured as the change in dose of concomitant corticosteroids, as the proportion of subjects achieving a dose of ≤5 mg/day oral prednisone or equivalent, as the proportion of subjects off prednisone, as the change in dose of SLE-related therapies, and as the proportion of subjects off all SLE-related therapy.

An additional secondary objective of this sub-study is to evaluate the effect of CAB-001 on patient-reported outcomes and health-related QOL at timepoints from Week 56 to Week 156 post-infusion, measured as the change from baseline in SF-36v2 Health Survey, Pain Numeric Rating Scale, FACIT-F, PtGA, Lupus QoL, and EQ-5D-5L scores.

An additional secondary objective of this sub-study is to evaluate the effect of CAB-001 on SLE serologies at timepoints from Week 56 to Week 156 post-infusion, measured as the changes from baseline in anti-dsDNA antibodies, and the changes from baseline in C3, C4, and CH50.

An additional secondary objective of this sub-study is to evaluate the safety and tolerability of CAB-001 in subjects with LN through 156 weeks, measured as AEs including AEs of special interest (AESI), vital signs, physical examination, and clinical laboratory tests.

Additional objectives and endpoints relate to a second sub-study, detailed in “Part B” below, which is conducted to further evaluate the efficacy of CAB-001 in the treatment of non-renal SLE.

A primary objective of this sub-study is to compare the efficacy between CAB-001 and belimumab plus standard of care at Week 52 in subjects with non-renal SLE in SRI-4 response, measured as the proportion of subjects achieving complete SRI-4 response at Week 52.

A secondary objective of this sub-study is to compare the efficacy of CAB-001 and belimumab plus standard of care in SRI-4 response, measured as: the proportion of subjects achieving complete SRI-4 response at Week 24, the proportion of subjects achieving complete SRI-4 response at the Weeks 24 and 52, the time to the first achievement of SRI-4 response, and the time to the first achievement of SRI-4 response and on no other SLE-related therapy.

An additional secondary objective of this sub-study is to compare the efficacy of CAB-001 and belimumab plus standard of care in SRI-6 response, measured as the proportion of subjects achieving SRI-6 response at Weeks 24 and 52, and as the proportion of subjects achieving SRI-6 response and on no other SLE-related therapy at Weeks 24 and 52.

An additional secondary objective of this sub-study is to compare the efficacy of CAB-001 and belimumab plus standard of care in BICLA response, measured as the proportion of subjects achieving BICLA response at Weeks 24 and 52.

An additional objective of this sub-study is to compare the efficacy of CAB-001 and belimumab plus standard of care in SLE disease activity, measured as the change from baseline in SLEDAI-2K score at Weeks 24 and 52, the change from baseline in BILAG-2004 scores at Weeks 24 and 52, the change from baseline in PGA scores at Weeks 24 and 52, the change from baseline in involved joint count at Weeks 24 and 52.

An additional objective of this sub-study is to compare the efficacy of CAB-001 and belimumab plus standard of care in skin activity, measured as the proportion of subjects achieving CLASI50 at Weeks 24 and 52.

An additional objective of this sub-study is to compare the efficacy of CAB-001 and belimumab plus standard of care in the achievement of low disease, measured as the proportion of subjects achieving Lupus Low Disease Activity State (LLDAS) at Weeks 24 and 52.

An additional objective of this sub-study is to compare the efficacy of CAB-001 and belimumab plus standard of care in the achievement of remission, measured as the proportion of subjects achieving Definition of Remission in SLE (DORIS) at Weeks 24 and 52.

An additional objective of this sub-study is to compare the effect of CAB-001 and belimumab plus standard of care in B cell count, measured as changes from baseline in peripheral B cell count over time through Week 52.

An additional objective of this sub-study is to compare the efficacy of CAB-001 and belimumab plus standard of care in SLE serology, measured as the changes from baseline in anti-dsDNA antibody level through Week 52 and as changes from baseline in C3, C4, and CH50 levels through Week 52.

An additional objective of this sub-study is to compare the efficacy of CAB-001 and belimumab plus standard of care in corticosteroid use, measured as the change from baseline in the dose of concomitant corticosteroids at Weeks 24 and 52, and as the proportion of subjects achieving a low dose of oral prednisone or equivalent at Weeks 24 and 52.

An additional objective of this sub-study is to compare the efficacy of CAB-001 and belimumab plus standard of care in disease flare, measured as incidence of mild/moderate and severe disease flare per BILAG-2004 and modified SELENA-SLEDAI Flare Index (SFI) through Week 52, and as time to mild/moderate and severe flare.

An additional secondary objective of this sub-study is to compare the effect of CAB-001 and belimumab plus standard of care in patient-reported outcomes and health-related QOL, measured as the change from baseline in SF-36v2, Pain NRS, FACIT-F, PtGA, Lupus QoL, and EQ-5D-5L scores at Week 24 and Week 52.

An additional objective of this sub-study is to evaluate the effect of CAB-001 on disease activity at response timepoints from Week 56 to Week 156 post-infusion, measured as the change from baseline in UPCR, eGFR, SLEDAI-2K, BILAG-2004, PGA, joint counts, CLASI, and SDI, and also measured as proportion of subjects achieving SRI-4, SRI-5, SRI-6 and BICLA responses and LLDAS and DORIS criteria.

An additional secondary objective of this sub-study is to evaluate the effect of CAB-001 on corticosteroid use and SLE-related therapy at time points from Week 56 to Week 156 post-infusion, measured as the change in dose of concomitant corticosteroids, as the proportion of subjects achieving a dose of ≤5 mg/day oral prednisone or equivalent, as the proportion of subjects off prednisone, as the change in dose of SLE-related therapies, and as the proportion of subjects off all SLE-related therapy.

An additional secondary objective of this sub-study is to evaluate the effect of CAB-001 on patient-reported outcomes and health-related QOL at timepoints from Week 56 to Week 156 post-infusion, measured as the change from baseline in SF-36v2 Health Survey, Pain Numeric Rating Scale, FACIT-F, PtGA, Lupus QoL, and EQ-5D-5L scores.

An additional secondary objective of this sub-study is to evaluate the effect of CAB-001 on SLE serologies at timepoints from Week 56 to Week 156 post-infusion, measured as the changes from baseline in anti-dsDNA antibody levels, and the changes from baseline in C3, CD4, and CH50 levels.

An additional secondary objective of this sub-study is to evaluate the safety and tolerability of CAB-001 in subjects with LN through 156 weeks, measured by AEs (including AEs of special interest), vital signs, physical examination, and clinical laboratory tests.

### Study Design

This is a phase 1/2 study designed to evaluate the safety, tolerability, and efficacy of CAB-001 in adult subjects with active SLE. The study has 2 parts. In Part A, CAB-001 single-arm treatment is evaluated for safety and tolerability, and a dose for use in Part B is identified. In Part B, CAB-001 is compared with belimumab to evaluate its safety and efficacy for the treatment of patients with LN and patients with active SLE who do not meet the criteria for LN (non-renal SLE) in 2 independent substudies. Any subject who receives CAB-001 is followed for 156 weeks for efficacy and safety.

### Part A: Open-Label Safety and Tolerability

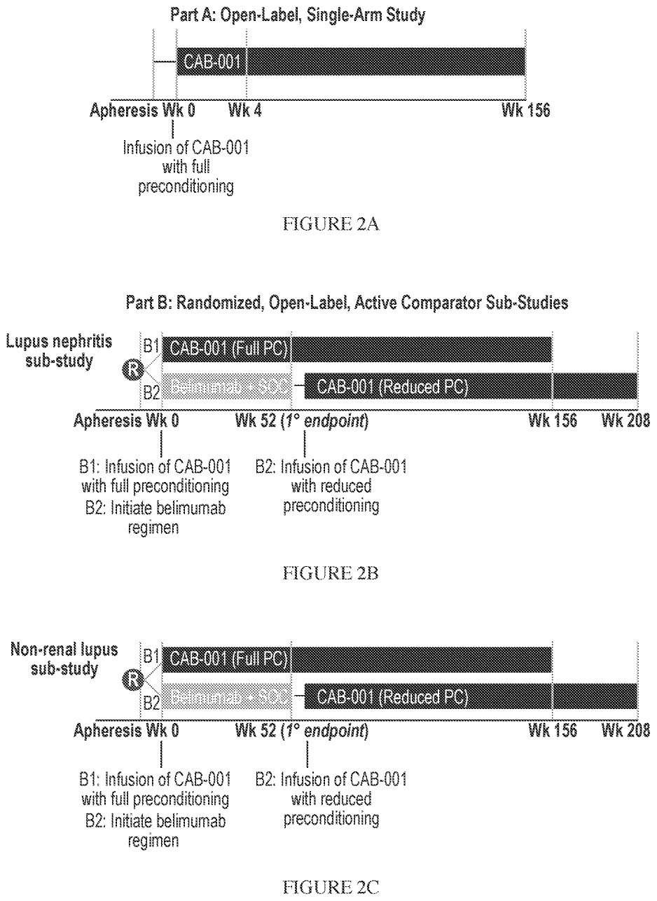

The design of Part A of the study is summarized in FIG. 2A. For Part A, a minimum of 6 subjects with either LN (minimum of 3 subjects) or SLE who do not fulfill the criteria for LN (“non-renal SLE”) are enrolled. All subjects undergo leukapheresis and the CAB-001 cell product is manufactured. Following leukapheresis and cell manufacturing, subjects are administered a standard preconditioning regimen: subjects are intravenously administered 25 mg/m²/day fludarabine on Day −5, Day −4, and Day −3, and subjects are intravenously administered 1,000 mg/m²/day cyclophosphamide on Day −3. On Day 0, subjects are infused with CAB-001 at a dose of 1×10⁶ CAR T cells/kg of weight (cohort A1). There is a minimum of 14 days between dosing of any 2 subjects, with 28 days between subject dosing if a dose-limiting toxicity (DLT) is observed. A DLT is defined as any of the following occurrences of AEs directly related to CAB-001: (1) Cytokine release syndrome (CRS): American Society for Transplantation and Cellular Therapy (ASTCT) Grade 2 CRS occurring within 28 days of cell infusion that fails to improve to Grade ≤1 within 7 days; (2) Immune effector cell-associated neurotoxicity syndrome (ICANS): ASTCT Grade 2 ICANS occurring within 28 days of cell infusion that fails to improve to Grade ≤1 or baseline within 7 days; (3) Common Terminology Criteria for Adverse Events (CTCAE) v5.0 Grade 2 organ toxicity (cardiac, dermatologic, gastrointestinal, hepatic, pulmonary, renal/genitourinary, or neurologic) that are clinically significant, not pre-existing, not due to disease progression, not related to other underlying medical condition(s), and occurring within 28 days of cell infusion that do not resolve to Grade ≤1 or baseline within 7 days; (4) any CRS Grade 3 or 4 or any ICANS Grade 3 or 4 occurring within 28 days of cell infusion; or (5) CTCAE Grade ≥3 organ toxicity (cardiac, dermatologic, gastrointestinal, hepatic, pulmonary, renal/genitourinary, or neurologic) that are clinically significant, not pre-existing, not due to disease progression, not related to other underlying medical condition(s), and occurring within 28 days of cell infusion. If a DLT is observed in 2 or more subjects, a lower dose of CAB-001 may be evaluated (e.g., cohort A0; see TABLE 5).

TABLE 5
CAB-001 Adaptive Dose Cohorts in Part A

| | Number | | | |
|---|---|---|---|---|
| Cohort/ | of | | | Dose |
| Group | Subjects | Fludarabine | Cyclophosphamide | (cells) |
| A1 | 3 | 25 mg/m² × 3 doses | 1,000 mg/m² × 1 dose | 1 × 10⁶ |
| A0 | 3 | 25 mg/m² × 3 doses | 1,000 mg/m² × 1 dose | 3 × 10⁵ |
| A2 | 3 | 25 mg/m² × 3 doses | 1,000 mg/m² × 1 dose | 3 × 10⁶ |
| A3 | 3 | 25 mg/m² × 3 doses | 1,000 mg/m² × 1 dose | 1 × 10⁷ |

With the observation of ≤1 DLT in the cohort, the decision to progress to Part B of the study is informed by translational biological response data at 28 days, including B cell depletion. If incomplete B cell depletion at 28 days is observed in 0 subjects, the study may progress to Part B without a change in CAR T cell dose. If incomplete B cell depletion at 28 days is observed in 1 subject, the dose of CAR T cells may be increased, or the study may progress to Part B at the current dose (e.g., as shown in TABLE 5, cohorts A2 and A3). If incomplete B cell depletion at 28 days is observed in 2 subjects, the dose may be increased.

All subjects in Part A are evaluated for safety, tolerability efficacy, and quality of life for 156 Weeks, as appropriate for assessing the study objectives.

### Part B: Active Comparator Sub-Studies

Two independent sub-studies are conducted to further evaluate the effect of CAB-001 compared to belimumab, a therapy approved for both LN and non-renal SLE, added to standard of care. Eligible subjects meeting the diagnostic criteria for LN are enrolled in the LN substudy, while other eligible subjects are enrolled in the non-renal SLE substudy. Approximately 66 subjects are enrolled in the LN sub-study and approximately 90 subjects are enrolled in the non-renal SLE sub-study for a total of approximately 156 subjects. The design of Part B of the study is summarized in FIGS. 2B-2C.

Subjects in each sub-study are randomized into at a ratio of 2:1 into two groups: Group B1 (CAB-001) and Group B2 (belimumab). Subjects in Group B1 undergo leukapheresis and CAB-001 CAR T cells are manufactured. Group B1 is intravenously administered a full preconditioning regimen of 25 mg/m²/day fludarabine on Day −5, Day −4, and Day −3, and 1,000 mg/m²/day cyclophosphamide on Day −3 (see TABLE 6). Group B1 is subsequently intravenously administered CAB-001 at the dose selected in Part A (between 3×10⁵ and 1×10⁷ CAR T cells/kg, e.g., 3×10⁵, 1×10⁶, 3×10⁶, or 1×10⁷ CAR T cells/kg) on Day 0. Subjects are evaluated for safety, tolerability, efficacy, and quality of life at regular visits through Week 52, as appropriate. After the Week 52 visit, subjects are followed and evaluated through Week 156, as appropriate.

Subjects in Group B2 undergo leukapheresis, and CAB-001 CART cells are manufactured. Subjects are intravenously administered a standard dose of belimumab for 12 months with standard of care. Subjects are evaluated for safety, tolerability, efficacy, and quality of life at regular visits through Week 52, as appropriate. At the conclusion of the Week 52 visit, subjects in Group B2 who meet the eligibility criteria are allowed to receive CAB-001 (“crossover subjects”). Contingent on emerging data from Part A, crossover subjects in Group B2 are administered a reduced preconditioning regimen of 12.5 mg/m²/day fludarabine on Day −5, Day −4, and Day −3, and 500 mg/m²/day cyclophosphamide on Day −3, in place of the full preconditioning regimen. Crossover subjects in Group B2 are subsequently administered CAB-001 at the dose used in Group B1. Crossover subjects are evaluated for safety, tolerability, efficacy, and quality of life for a total of 156 weeks after the infusion.

TABLE 6
CAB-001 Adaptive Dose Cohorts in Part B

| | Cyclophosphamide (CY) + | | | | | |
|---|---|---|---|---|---|---|
| | Fludarabine (FLU) Preconditioning | | | | | |
| | Regimen (doses mg/m²/day) | | | | | Dose |
| Cohort/Group | Day −5 | Day −4 | Day −3 | Day −2 | Day −1 | (cells) |

| | | | | |
|---|---|---|---|---|
| B1 (CAB-001 with full | 25 | 25 | 25 | TBD based |
| preconditioning) | FLU | FLU | FLU; | on results |
| | | | 1,000 | in Part A |
| | | | CY | |
| B2 (12 mo. Belimumab, | 12.5 | 12.5 | 12.5 | TBD based |
| with potential to follow | FLU | FLU | FLU; | on results |
| with CAB-001 with reduced | | | 500 | in Part A |
| preconditioning) | | | CY | |

### Duration of Study

For subjects in Part A and Part B, Group B1, the pre-treatment period, which includes screening, leukapheresis (cell harvesting), cell manufacturing, pre-conditioning, and pre-infusion (baseline) assessments, lasts between approximately 4 and 18 weeks. CAB-001 infusion lasts about 1 day. The post-treatment period lasts 156 weeks and includes follow-up visits for assessments necessary for the study objectives.

For subjects in Part B, Group B2, the pre-treatment period, which includes screening and leukapheresis (cell harvesting), lasts up to 14 weeks. Treatment with belimumab lasts for approximately 52 weeks. For crossover subjects, this is followed by preconditioning, CAB-001 treatment, and post-infusion follow-up, which lasts up to about 162 weeks.

After the completion of the maximum 156-week post-treatment period, all subjects who have received CAB-001 will be followed in the long-term follow-up (LTFU) period. In the LTFU period, subjects are followed for a maximum of 15 years after CAB-001 infusion (not including the pre-infusion periods and belimumab treatment, if any).

### Inclusion/Exclusion Criteria

Inclusion criteria for the Part A of the study are: (1) able to provide informed consent; (2) a minimum of 18 and a maximum of 65 years of age; (3) Eastern Cooperative Oncology Group (ECOG) performance status 0-1; (4) a clinical diagnosis of SLE, based on the 2019 The European League Against Rheumatism (EULAR)/American College of Rheumatology (ACR) classification criteria for adult SLE; (5) positive anti-nuclear antibody (ANA) titer ≥1:80 or positive anti-dsDNA antibody at screening; (6) for LN subjects, have active, biopsy-proven lupus nephritis class III or IV, with or without the presence of Class V, using the 2003 ISN/RPS criteria (the biopsy must be performed within 6 months prior to the screening visit or during the screening period); (7) diagnosed with active SLE as defined by the following criteria: a. for LN subjects: UPCR≥1 mg/mg on 2 first morning void urine samples during screening despite prior or current treatment with standard of care therapy, including corticosteroids and either mycophenolate mofetil (MMF)/mycophenolic acid (MPA) or cyclophosphamide, or b. for non-renal SLE subjects: SLEDAI-2K≥8 and clinical SLEDAI-2K≥6 (excluding headache, alopecia, mucosal ulcers, fever, and organic brain syndrome) during screening despite prior or current treatment with standard of care therapy, including corticosteroids, rituximab or other B cell depleting agents, cyclophosphamide, MMF/MPA, azathioprine, methotrexate, 6-mercaptopurine, sirolimus, tacrolimus, thalidomide, leflunomide, mizorbine, anifrolumab, and belimumab; (8a) if currently receiving standard immunosuppressive therapy (including MMF/MPA, oral cyclophosphamide, azathioprine, antimalarial therapy, methotrexate, 6-mercaptopurine, sirolimus, tacrolimus, thalidomide, leflunomide, or mizorbine), the therapy must have been initiated at least 12 weeks prior to screening and on a stable dose for at least 8 weeks prior to screening, except for dose reduction due to safety or tolerability; (8b) if currently receiving a renin-angiotensin-aldosterone inhibitor, (including direct renin inhibitors, angiotensin converting enzyme (ACE) inhibitors, angiotensin receptor blockers (ARBs), and mineralocorticoid receptor blockers), the subject must be on a stable dose for at least 2 weeks prior to screening; (8c) if currently receiving an oral corticosteroid, must be receiving ≤30 mg of prednisone or equivalent on a stable dose for at least 2 weeks prior to screening; (9) estimated glomerular filtration rate of >30 mL/min/1.73 m² using the chronic kidney disease-epidemiology equation; (10) adequate hepatic function as evidenced by alanine aminotransferase (ALT)≤2.5×ULN; (11) in the opinion of the investigator, subject requires additional systemic therapy for SLE and is a reasonable candidate for CAB-001 therapy; and (12) women of childbearing potential (WOCBP) must agree to use birth control from screening to a minimum of 52 weeks after the CAB-001 infusion (acceptable contraception methods include intrauterine device, hormone-based contraception, true sexual abstinence, partner vasectomy, or monogamous relationship with a male partner with azoospermia).

Exclusion criteria for Part A of the study are: (1) treatment with rituximab or other B cell-depleting agent within 6 months prior to screening, or within 3 months if there are laboratory results indicating presence of CD19+ B cells; (2) treatment with voclosporin or other calcineurin inhibitor within 2 months prior to screening; (3) treatment with anifrolumab or other biologic agent within 3 months prior to screening; (4) for LN subjects only, greater that 50% of glomeruli with sclerosis on kidney biopsy; (5) diagnosis of cancer, except basal or squamous cell skin cancer or carcinoma in situ of the cervix that has been excised and cured and at least 5 years since excision; (6) planned major surgery (including joint surgery) within 52 weeks following the CAB-001 infusion; (7) contraindication to leukapheresis; (8) history of anaphylactic or severe systemic reaction to fludarabine, cyclophosphamide, or any of their metabolites; (9) absolute lymphocyte count <1,000/μL at screening; (10) positive human immunodeficiency virus (HIV), hepatitis C antibody, hepatitis B surface antigen test, or evidence of active or chronic tuberculosis (TB) at screening; (11) active infection requiring medical intervention at screening; (12) autoimmune disorder other than SLE requiring immunosuppressive therapies; (13) the presence of kidney disease other than active lupus nephritis; (14) current symptoms of severe, progressive, or uncontrolled renal, hepatic, hematological, gastrointestinal, pulmonary, psychiatric, cardiac, neurological, or cerebral disease, including severe and uncontrolled infections, such as sepsis and opportunistic infections; (15) concomitant medical conditions that, in the opinion of the investigator, might place the subject at unacceptable risk for participation in this study, interfere with the assessment of the effects or safety of the investigational product or with the study procedures; (16) previous CAR T cell therapy; (17) prior solid organ (heart, liver, kidney, lung) transplant of hematopoietic cell transplant; (18) live vaccine within 30 days of pre-infusion visit; (19) pregnant or lactating woman, or plan to become pregnant within 52 weeks following the CAB-001 infusion; or (20) unable or unwilling to comply with protocol.

Inclusion criteria for Part B of the study are: (1) able to provide informed consent; (2) minimum of 18 years of age and maximum of 65 years of age; (3) Eastern Cooperative Oncology Group (ECOG) performance status 0-1; (4) a clinical diagnosis of SLE, based on the 2019 The European League Against Rheumatism (EULAR)/American College of Rheumatology (ACR) classification criteria for adult SLE; (5) positive ANA≥1:80 or positive anti-dsDNA antibody; (6) for LN subjects, have active, biopsy-proven lupus nephritis class III or IV, with or without the presence of Class V, using the 2003 ISN/RPS criteria (the biopsy must be performed within 6 months prior to the screening visit or during the screening period); (7) subjects are diagnosed with active SLE, further wherein subjects with LN or non-renal SLE should meet the following criteria: a. for LN substudy: UPCR≥1 mg/mg on 2 first morning void urine samples during screening despite treatment with standard of care therapy with glucocorticoids and either MMF/MPA or cyclophosphamide, or b. for non-renal SLE substudy: SLEDAI-2K≥8 and clinical SLEDAI-2K≥6 (excluding headache, alopecia, mucosal ulcers, fever, and organic brain syndrome) during screening despite prior or current treatment with standard of care therapy, including corticosteroids, rituximab or other B cell depleting agents, cyclophosphamide, MMF/MPA, azathioprine, 6-mercaptopurine, methotrexate, sirolimus, tacrolimus, thalidomide, leflunomide, mizorbine, and anifrolumab; (8a) if receiving standard immunosuppressive therapy (including MMF/MPA, oral cyclophosphamide, azathioprine, 6-mercaptopurine, antimalarial, methotrexate, sirolimus, tacrolimus, thalidomide, leflunomide, or mizorbine), the therapy must have been initiated at least 12 weeks prior to screening and at a stable dose for at least 8 weeks prior to screening, except for dose reduction due to safety or tolerability; (8b) if receiving a renin-angiotensin-aldosterone inhibitor, (including direct renin inhibitors, ACE inhibitors, ARBs, and mineralocorticoid receptor blockers), the subject must be on a stable dose for at least 2 weeks prior to screening; (8c) if receiving an oral corticosteroid, the subject must be receiving ≤30 mg prednisone or equivalent and on a stable dose for at least 2 weeks prior to screening; (9) estimated glomerular filtration rate of ≥30 mL/min/1.73 m² using the chronic kidney disease-epidemiology equation; (10) adequate hepatic function as evidenced by ALT≤2.5×ULN; (11) in the opinion of the investigator, subject requires additional systemic therapy for SLE and is a reasonable candidate for CAB-001 therapy; and (12) WOCBP must agree to use birth control from screening to a minimum of 52 weeks after the CAB-001 infusion or 16 weeks after the last belimumab infusion, as applicable (acceptable contraception methods include intrauterine device, hormone-based contraception, true sexual abstinence, partner vasectomy, or monogamous relationship with a male partner with azoospermia).

Exclusion criteria for Part B of the study are: (1) treatment with rituximab or other B cell-depleting agent within 6 months prior to screening, or 3 months if there are laboratory results indicating presence of CD19+ B cells; (2) prior treatment with belimumab; (3) treatment with voclosporin or other calcineurin inhibitor within 2 months prior to screening; (4) treatment with anifrolumab or other biologic agent within 3 months prior to screening; (5) for LN substudy, greater than 50% of glomeruli with sclerosis on kidney biopsy; (6) diagnosis of cancer except basal or squamous cell skin cancer or carcinoma in situ of the cervix that has been excised and cured and at least 5 years since excision; (7) planned major surgery (including joint surgery) during the 52 weeks following the CAB-001 or the initial belimumab infusion; (8) contraindication to leukapheresis; (9) for LN substudy, history of intolerance, anaphylactic or severe systemic reaction to MMF/MPA; (10) history of anaphylactic or severe systemic reaction to fludarabine, cyclophosphamide, or any of their metabolites; (11) absolute lymphocyte count <1,000/μL at screening; (12) positive test for HIV, hepatitis C antibody, hepatitis B surface antigen, or evidence of active or chronic TB at screening; (13) active infection requiring medical intervention at screening; (14) autoimmune disorder other than SLE requiring immunosuppressive therapies; (15) the presence of kidney disease other than active lupus nephritis; (16) current symptoms of severe, progressive, or uncontrolled renal, hepatic, hematological, gastrointestinal, pulmonary, psychiatric, cardiac, neurological, or cerebral disease, including severe and uncontrolled infections, such as sepsis and opportunistic infections; (17) concomitant medical conditions that, in the opinion of the investigator, might place the subject at unacceptable risk for participation in this study, interfere with the assessment of the effects or safety of the investigational product or with the study procedures; (18) previous CAR T cell therapy; (19) prior solid organ (heart, liver, kidney, lung) transplant of hematopoietic cell transplant; (20) live vaccine received within 30 days of pre-infusion visit; (21) pregnant or lactating woman, or plan to become pregnant within 52 weeks following the CAB-001 infusion or the initial belimumab infusion; or (22) unable or unwilling to comply with protocol.

### Example 4

This example describes treating an autoimmune disease in a subject in need thereof by administering T cells engineered to express a CAR, after administration of a preconditioning regimen comprising a dose of cyclophosphamide and a dose of fludarabine, wherein the dose of cyclophosphamide and/or the dose of fludarabine is reduced as compared to a standard preconditioning regimen.

T cells are obtained from a subject having an autoimmune disease (for example, an autoimmune disease selected from (SLE), lupus nephritis, SLE with anti-dsDNA antibodies, pemphigus vulgaris (PV), mucosal PV, mucocutaneous PV, myasthenia gravis (MG), MuSK-associated MG, AChR MG, myositis, membranous nephropathy, anti-synthetase syndrome, dermatomyositis, immune mediated necrotizing myopathy, multiple sclerosis, neuromyelitis optica, chronic inflammatory demyelinating polyneuropathy, anti-NMDA Receptor encephalitis, Lambert-Eaton syndrome, pemphigus foliaceus, epidermolysis bullosa acquisita, bullous pemphigoid, Goodpasture's syndrome, rheumatoid arthritis, systemic sclerosis, anti-neutrophil cytoplasmic antibody (ANCA)-associated vasculitis, immune thrombocytopenic purpura, antiphospholipid syndrome, autoimmune hemolytic anemia, type 1 diabetes, Grave's disease, Hashimoto's disease, and Sjogren's syndrome). The T cells are transduced with a vector encoding an anti-CD19 CAR, e.g., an anti-CD19 CAR comprising a CD8α transmembrane domain, a 4-1BB costimulatory domain, and a CD3ζ intracellular domain, e.g., a CAR comprising the amino acid sequence of SEQ ID NO: 23. The transduced T cells are expanded and, optionally, cryopreserved.

Prior to CAR T cell infusion, the subject is administered a reduced-dose preconditioning regimen comprising an intravenous dose of cyclophosphamide and an intravenous dose of fludarabine. For example, the subject may be administered a preconditioning regimen as disclosed in TABLE 1. A control subject or control group may instead be administered a standard preconditioning dose, e.g., a total dose of 1,000 mg/m² cyclophosphamide and a total dose of 75-90 mg/m² fludarabine.

A dose of CAR T cells (e.g., from 1×10⁶ to 1×10⁷ cells/kg bodyweight) is intravenously administered to the subject. The subject is monitored post-infusion for B-cell levels, white blood cell count, CAR T cell levels, and for autoimmune disease markers (e.g., autoantibody levels).

It is contemplated that the autologous CD19-CAR T cell therapy is effective to treat the autoimmune disease. It is further contemplated that the use of the reduced preconditioning regimen prior to treatment with CD19-CAR T cell therapy is effective in the treatment of the autoimmune disease, and that a standard, full-dose preconditioning regimen is not required.

### Example 5

This example describes the treatment of systemic sclerosis in a patient by administering T cells engineered to express a CD19-specific CAR, after administration of a preconditioning regimen comprising reduced doses of cyclophosphamide and fludarabine.

A patient having systemic sclerosis was treated using an autologous CD19-specific CAR T cell therapy using the therapeutic protocol described in Example 4. This protocol was also substantially the same as the treatment protocol described in Example 1, except a different autoimmune indication was treated. Briefly, autologous T cells were obtained from the patient and transduced with a lentiviral anti-CD19 CAR vector, which encoded an anti-CD19-CAR construct essentially comprising an scFv which specifically binds CD19, a CD8α transmembrane domain, a 4-1BB costimulatory domain, and a CD3ζ signaling domain. The anti-CD19 scFv comprised the amino acid sequence of SEQ ID NO: 18. The patient was administered a reduced-dose preconditioning regimen prior to CAR T cell infusion: a dose of 12.5 mg/m²/day fludarabine was administered on Days −5, −4, and −3, and a dose of 500 mg/m²/day cyclophosphamide was administered on Day −3. (See TABLE 1, Regimen 1). A dose of 1×10⁶ CAR T cells per kg bodyweight was administered to the patient as a single intravenous infusion on Day 0. The patient was monitored post-infusion for systemic sclerosis disease markers, B-cell levels, and CAR T cell levels.

The CAR T cell population successfully expanded in the subject post-infusion, in spite of the reduced preconditioning regimen. CAR-T cells expanded from Day 3 to Day 9 post-infusion. CAR T levels decreased after Day 9, but were still detectable (1%) at Day 119. B cells were completely depleted by Day 7 and were not detectable until approximately Day 80. Serum-IgG levels remained above 700 mg/dL. Anti-nuclear antibodies and anti-RNA polymerase III antibodies were no longer detectable at 3 and 6 months after CAR T cell infusion. Myocardial tracer uptake was reduced by 32.6% (using 68Ga-FAPI-04-PET-CT imaging, a novel imaging technique that allows the molecular assessment of fibroblast activation in vivo). Echocardiography performed 6 months after CAR T cell therapy demonstrated that the left ventricular ejection fraction remained stable while signs of right ventricular strain showed a trend towards improvement. The extent of lung fibrosis on computerized tomography scans at 3 months and pulmonary function test parameters at 3 and 6 months was stable or slightly improved. As analyzed by magnetic resonance imaging, carpal arthritis improved 3 months after therapy. Consistently, tender joint counts improved from 22 at baseline to 3 joints at 3- and 6-month follow-up assessments. In addition, tendon friction rubs were no longer present, resulting in a reduction in the European Scleroderma Trials and Research Group activity index score. Skin fibrosis showed a tendency toward improvement after 3 months, as analyzed by 2 independent assessors. The treatment was well tolerated with grade 1 (mild fever for less than 24 hours) cytokine release syndrome (CRS) and no signs of immune effector cell-associated neurotoxicity syndrome (ICANS).

Collectively, these data demonstrate that the use of a full-dose preconditioning regimen may not be necessary for treatment of systemic sclerosis with CD19-CAR T cell therapy, and that the use of a reduced-dose preconditioning regimen (a total dose of 500 mg/m² cyclophosphamide and 37.5 mg/m²) may also be effective for treatment of systemic sclerosis with CD19-CAR T cell therapy.

### Example 6

This example describes a Phase 1/2, open-label study designed to evaluate the safety and efficacy of CAB-001 in participants with generalized myasthenia gravis.

### Background

Myasthenia gravis (MG) is a rare autoimmune disorder characterized by autoantibody responses that cause defective transmission of signals at the neuromuscular junction, resulting in a distinctive pattern of weakness. In approximately 15% of patients with MG, symptoms are limited to the ocular muscles, termed ocular MG (Gilhus et al. (2019) NAT. REV. DIS. PRIMERS 5(1): 30). Patients with generalized MG typically experience symptoms associated with ocular disease in addition to weakness of many other voluntary muscle groups, including extremity, bulbar, and respiratory muscles (Gilhus et al, supra). Profound weakness from MG can have devastating consequences on a patient's quality of life, including dysarthria, dysphagia, impaired vision, shortness of breath, and impaired mobility, as well as episodes of pulmonary failure requiring mechanical ventilation, termed myasthenic crises (Engel-Nitz et al. (2018) MUSCLE NERVE 1(1): 16-22; Wendell and Levine (2011) NEUROHOSPITALIST 1(1): 16-22).

MG is considered a classic example of a B cell-mediated autoimmune disease, in which the majority of patients have autoantibodies directed to molecular elements of the neuromuscular junction, causing disruption of receptor signaling and potentially complement-mediated tissue damage (Yi et al. (2018) MUSCLE NERVE 57(2): 172-184). In approximately 85% of patients, these autoantibodies are directed against the muscle acetylcholine receptor (AChR). Less commonly identified autoantibodies include those targeted to muscle-specific tyrosine kinase (MuSK) and low-density lipoprotein receptor-related protein 4 (LRP4) in 6% and 2% of MG patients, respectively (Dresser et al. (2021) J. CLIN. MED. 10(11): 2235). Currently, there are no curative treatments for MG. Available therapeutic options for MG have limited effectiveness, focus solely on specific symptoms, and are associated with serious long-term side effects.

### Investigational Product

The investigational product, CAB-001, is an autologous CD19-targeting chimeric antigen receptor (CAR) T cell. The CAR comprises a fully human scFv which specifically binds CD19, a CD8α transmembrane domain, a 4-1BB costimulatory domain, and a CD3ζ signaling domain. The anti-CD19 scFv comprises the amino acid sequence of SEQ ID NO: 9, and the construct comprises the amino acid sequence of SEQ ID NO: 23 (or SEQ ID NO: 27, including a CD8α signal peptide).

### Objectives and Endpoints

A primary objective of this study is to evaluate the safety and tolerability of CAB-001 in participants with generalized MG, measured by incidence of adverse events (AEs) occurring within 28 days after CAB-001 infusion.

An additional objective of this study is to evaluate the safety and tolerability of CAB-001 in participants with generalized MG over 156 weeks, measured by AEs, vital signs, physical exam findings, and clinical laboratory test results occurring within 156 weeks after CAB-001 infusion.

An additional objective of this study is to evaluate the effects of CAB-001 on hematologic and immunologic parameters including WBCs and T, B, NK, and myeloid populations, measured as changes from baseline in WBC with differential, as well as T, B, NK cell counts and immunophenotypic sub-populations after CAB-001 infusion.

An additional objective of this study is to evaluate CAB-001 persistence and kinetics after infusion, measured by the number and percentage of CAB-001 positive cells in peripheral blood of participants overtime.

An additional objective of this study is to evaluate the effects of CAB-001 on MG serology, measured, for participants with MG-specific autoantibodies (e.g., anti-AChR, anti-MuSK, anti-LRP4), as the change from baseline in autoantibody levels and/or status.

An additional objective of this study is to evaluate the effects of CAB-001 on generalized MG disease activity, measured as the change from baseline in MG-ADL, QMG, MGC, MGFA-PIS, and MGII, and as the proportion of participants achieving MSE (MG-ADL score of 0 or 1), MG-ADL response (≥2.0-point improvement from baseline), QMG response (≥3.0-point improvement from baseline), and MGC response (≥3.0-point improvement from baseline).

An additional objective of this study is to evaluate the time to achieve disease response after CAB-001, measured as time to achieve MSE on MG-ADL response, QMG response, and MGC response.

An additional objective of this study is to evaluate the effect of CAB-001 on patient reported outcomes, measured as change in MG-QOL 15r, Neuro-QoL Fatigue, and/or EQ-5D-5L.

An additional objective of this study is to evaluate the effect of CAB-001 on disease flare, measured as (1) incidence of clinical deterioration, including use of rescue therapy and MG crisis; (2) incidence of hospitalizations and MG-related hospitalizations, including hospital days, ICU days, and ventilatory support days; and (3) time to clinical deterioration.

An additional objective of this study is to evaluate the effect of CAB-001 on drug-free response, measured as the proportion of participants achieving drug-free minimal symptom expression, MG-ADL response, QMG response, and/or MGC response.

An additional objective of this study is to evaluate the time to achieve drug-free response after CAB-001 infusion, measured as time to achieve drug-free minimal symptom expression, MG-ADL response, QMG response, and/or MGC response.

### Study Design

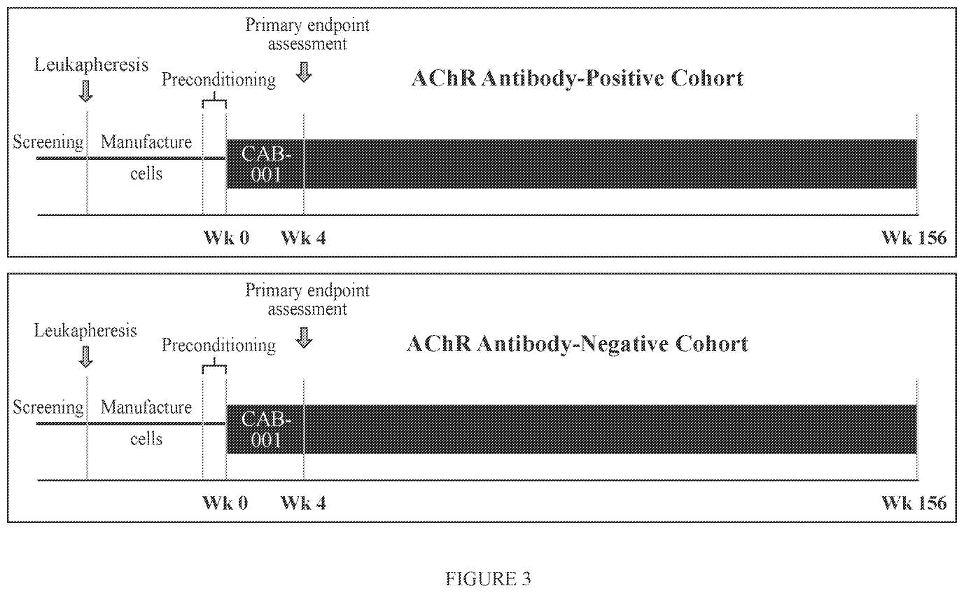

The design of this study is summarized in FIG. 3. The study includes participants with generalized MG who are diagnosed with Myasthenia Gravis Foundation of America class (MGFA) II, III, or IV. Two cohorts of participants will be studied based upon MG-identified autoantibody status: (1) Acetylcholine Receptor (AChR) Antibody-Positive Cohort, which includes participants with autoantibodies directed to AChR based on a historical record or test at Screening; and (2) AChR Antibody-Negative Cohort, which includes participants with negative results for autoantibodies directed to AChR based on a historical record and test at time of screening. For cohort (2), participants must have autoantibodies directed to muscle-specific tyrosine kinase (MuSK) or low-density lipoprotein receptor-related protein 4 (LRP4) based on a historical record or test at time of screening, or seronegative MG.

Each participant undergoes leukapheresis, and the CAB-001 cell product for the participant is manufactured. Briefly, autologous T cells are genetically modified ex vivo by transduction with a lentivirus vector. Fresh leukapheresis is collected and cryopreserved in a few aliquots. One frozen aliquot is used for one manufacturing production. Upon completion of the manufacturing process, the drug product is formulated and cryopreserved.

Following leukapheresis and cell manufacturing, participants are administered a preconditioning regimen. The preconditioning regimen may be a standard preconditioning regimen (e.g., IV Fludarabine administered at 25 mg/m² on Days −5, −4 and −3, and IV Cyclophosphamide administered at 1,000 mg/m² on Day −3) or may be a preconditioning regimen comprising a reduced dose of cyclophosphamide and/or fludarabine (e.g., as described in TABLE 1). CAB-001 is administered via IV infusion to participants at one of the four following doses: 1×10⁶ cells/kg, 3×10⁵ cells/kg, 3×10⁶ cells/kg, 1×10⁷ cells/kg.

Participants are evaluated for toxicities related to the CAB-001 therapy and its effect on their underlying disease during an initial 28-day assessment period. Participants are monitored in the days post-infusion, and subsequent visits occur approximately on Days 5, 8, 15, 22, and 29 post-infusion. In addition to routine chemistry and hematology tests, lab tests for PK sampling, coagulation panel, CRS labs, serum cytokine assessment, and CAR T assessment are performed through Day 29. After the Day 29 post-infusion evaluation, participants undergo evaluations every 4 weeks (±7 days) in the first year, approximately every 12 weeks (+14 days) in the second year, and approximately once every 26 weeks (±30 days) in the third year. All participants are evaluated for safety, tolerability, efficacy, and quality of life for 156 weeks following infusion. Participants may also monitored for 15 years after treatment, as appropriate.

### Inclusion Exclusion Criteria:

Inclusion criteria for participation in this study include: (1) able to provide informed consent; (2) be 18 or older and 70 or younger; (3) have MG with generalized muscle weakness meeting criteria as defined by the MGFA class II, III, IVa, and IVb, with diagnosis supported by meeting at least 1 of the following 2 criteria: a. positive serologic test for anti-AChR, anti-MuSK, or anti-LRP4 antibodies, based on historical record or test at screening, or b. evidence of response to acetylcholinesterase inhibitors and history of abnormal neuromuscular transmission demonstrated by repetitive nerve stimulation or single-fiber electromyography, wherein any seronegative cases have negative genetic testing for congenital myasthenic syndrome, and, in those with only abnormal jitter, mitochondrial disease excluded; (4) a total Myasthenia Gravis Activities of Daily Living (MG-ADL) score of ≥6 points during screening despite prior or current standard of care therapy for at least 12 weeks with at least two prior treatments, including cholinesterase inhibitors, corticosteroids, non-steroidal immunosuppressive drugs, B cell depleting therapies (e.g., rituximab), FcRn inhibitors (e.g., efgartigimod, rozanolixizumab), complement inhibitors (e.g., eculizumab, ravulizumab, zilucoplan), IVIg or subcutaneous immunoglobulin therapy, immunoadsorption, or plasma exchange; (5) if a patient is currently receiving standard therapy for MG, that treatment is required to be at a stable dose prior to screening as follows, except for dose reduction due to safety or tolerability (a. acetylcholinesterase inhibitors: Stable dose for ≥2 weeks; b. corticosteroids: ≤30 mg prednisone or equivalent and on a stable dose for ≥2 weeks; c. non-steroidal immunosuppressive drugs (e.g., azathioprine, methotrexate, cyclosporine, tacrolimus, mycophenolate mofetil/mycophenolic acid, CY) initiated at least ≥12 weeks prior to Screening, with stable dose for ≥4 weeks); (6) have received all currently recommended vaccinations per Centers for Disease Control and Prevention (CDC) or institutional guidelines for immunocompromised individuals before or during screening, including COVID-19/severe acute respiratory syndrome coronavirus 2 (SARS-CoV-2) (live vaccines must be administered at least 30 days prior to the Pre-Infusion Visit, and non-live vaccines should be administered to participants at least 2 weeks prior to the start of study drug infusion and, if possible, non-live vaccines should also be administered at least 2 weeks prior to leukapheresis; (7) clinical stability by vital signs assessment at the time of screening, including: a. systolic blood pressure ≥90 mmHg and ≤170 mmHg, b. diastolic blood pressure ≥55 mmHg and ≤10⁵ mmHg c. pulse ≥50 and ≤110 beats per minute, d. respiratory rate ≥12 and ≤20 breaths per minute, and e. afebrile; (8) women of reproductive potential must agree to use 2 acceptable methods of birth control from screening to a minimum of 52 weeks after the CAB-001 infusion; and (9) sexually active men who are reproductively competent, including vasectomized men (who do not have confirmation of infertility by laboratory testing), who are to receive CAB-001 are required to use condoms with spermicide from screening until at least 52 weeks after CAB-001 infusion.

Exclusion criteria include patients you have any of the following criteria: (1) MGFA class I or V; (2) patients judged by the Investigator to be inappropriate for the study (e.g., at risk for requiring intubation during the study); (3) active or untreated thymoma, history of thymic carcinoma or thymic malignancy; (4) history of thymectomy within 52 weeks of screening; (5) clinical evidence of other serious concomitant medical disease, worsening muscle weakness secondary to concurrent infections or medications, or recent or planned major surgery, which could confound the results of the trial or place the participant at undue risk; (6) use of the following therapies within the time period specified below: a. Eculizumab within 12 weeks prior to screening, zilucoplan within 5 weeks of screening, or ravulizumab within 40 weeks prior to screening, unless complement testing results (i.e., the total hemolytic complement [CH50] measurement) are normal at the time of screening, b. rituximab or other B cell-depleting agent within 26 weeks prior to screening, or within 12 weeks prior to screening if there are laboratory results indicating presence of CD19+ B cells at the time of screening, and c. any investigational agent within 4 weeks or 5 half-lives of screening, whichever is longer; (7) contraindications to leukapheresis; (8) history of anaphylactic or severe systemic reaction to fludarabine, cyclophosphamide, or any of their metabolites; (9) positive human immunodeficiency virus (HIV), hepatitis C virus (HCV) antibody, or hepatitis B surface antigen test, or evidence of active or chronic tuberculosis at screening; (10) any of the following clinical laboratory values at screening: a. Absolute lymphocyte count <500/μL, b. Absolute neutrophil count <1,000/μL, c. Hemoglobin <8 g/dL, d. Platelet count <50,000/μL, (11) active infection requiring medical intervention at screening; (12) active, inflammatory autoimmune disorder other than MG requiring immunosuppressive therapies, or other neurologic or neuromuscular disorder, that would interfere with an accurate assessment of clinical symptoms; (13) known malignancy or a history of malignancy, with the exception of local basal or squamous cell carcinoma of the skin or carcinoma in situ of the uterine cervix, with no evidence of recurrence for ≥3 years before screening; (14) current symptoms of severe, progressive, or uncontrolled renal, hepatic, hematological, gastrointestinal, pulmonary, psychiatric, cardiac, concomitant neurological, or cerebral disease, including severe and uncontrolled infections, such as sepsis and opportunistic infections; (15) chronic pulmonary disease, including: a. Resting oxygen saturation <90% without supplemental oxygen, b. Forced vital capacity (FVC)<60%, if measured; (16) impaired cardiac function or clinically significant cardiac disease, including: a. Unstable angina or myocardial infarction or coronary artery bypass graft within 6 months prior to leukapheresis, b. New York Heart Association stage III or IV congestive heart failure, c. history of clinically significant cardiac arrhythmia (e.g., ventricular tachycardia), complete left bundle branch block, or high-grade atrioventricular block, d. history of severe nonischemic cardiomyopathy, and e. left ventricular ejection fraction <45%, as assessed by echocardiogram or multi-gated acquisition scan (if performed)≤8 weeks of leukapheresis; (17) prior engineered T cell therapy involving permanent gene modification; (18) prior solid organ (heart, liver, kidney, lung) transplant or hematopoietic stem cell transplant; (19) pregnant or lactating women; (20) unwilling or unable to comply with the protocol.

It is contemplated that the CD19-CAR T cell therapy will be effective to treat myasthenia gravis. It is further contemplated that the use of the reduced preconditioning regimen prior to treatment with CD19-CAR T cell therapy will be effective in the treatment of myasthenia gravis, and that a standard, full-dose preconditioning regimen is not required.

### Example 7

This example describes a Phase 1/2, open-label study designed to evaluate the safety and efficacy of CAB-001 in adult participants with systemic sclerosis.

### Background

Systemic sclerosis (SSc) is a rare, heterogeneous, and potentially fatal multisystem chronic autoimmune disorder. It is characterized by progressive skin and internal organ fibrosis, diffuse fibroproliferative vasculopathy, and autoimmunity (Truchetet et al. (2023) CLIN. REV. ALLERGY IMMUNOL. 64(3): 262-283). Clinical presentations and the course of an individual patient's SSc are highly heterogeneous, and life expectancy is variably affected, mostly by the degree of lung and heart involvement.

Patients with SSc are classified based on the extent of their skin involvement into limited cutaneous systemic sclerosis (lcSSc) and diffuse cutaneous systemic sclerosis (dcSSc), also known as diffuse systemic sclerosis. Approximately one-third of patients are diagnosed with the more severe form of SSc, dcSSc (Jaafar et al. (2021) ARTHRISIS RES. THER. 23(1): 170). In lcSSc, skin fibrosis is restricted to the fingers (sclerodactyly), distal extremities, and face; in dcSSc, the trunk and proximal extremities are also affected. In patients with lcSSc, Raynaud's phenomenon typically precedes skin involvement and other disease manifestations by months to years (Allanore et al. (2015) NAT. REV. DIS. PRIMERS 1: 15002). Patients with dcSSc have a poorer prognosis with extensive skin changes and rapid disease progression that may involve all areas of the body, in which progression from Raynaud's phenomenon to skin thickening may occur within 1 year and internal organ vasculopathy and fibrosis within 5 years of diagnosis, including the gastrointestinal tract, heart, lungs, and kidneys. Patients with dcSSc have a disease-related mortality of 5% to 12% per year (Allanore, supra; Milanetti et al. (2011) CURR. STEM CELL. RES. THER. 6(1): 16-28).

The involvement of pro-inflammatory cytokines (such as interleukin [IL]6, IL 13, and tumor necrosis factor [TNF]) and transforming growth factor-β, as well as the presence of autoantibodies are helpful in SSc for both diagnosis and classification. Antinuclear antibody (ANA) is positive in approximately 90% of SSc cases. Anti-centromere and anti-Th/To antibodies are more commonly seen in lcSSc than in dcSSc. Anti-topoisomerase I antibody and anti-ribonucleic acid (RNA) polymerase III antibody is more often associated with dcSSc (Allanore et al., supra; Cavazzana et al. (2023) CLIN. REV. ALLERGY IMMUNOL. 64(3): 412-430; Truchetet, supra; Yang et al. (2020) FRONT. MED. (LAUSANNE) 7:587773). Anti-U3-RNP (fibrillarin) antibody is associated with a well-defined clinical phenotype and overall poor prognosis (male patients, Afro-Caribbean descent, younger at diagnosis, and higher risk of internal organ involvement, such as pulmonary arterial hypertension [PAH] and gastrointestinal and cardiac involvement) (Cavazzana, supra).

Interstitial lung disease (ILD) and pulmonary hypertension together account for nearly 50% of the deaths, followed by renal and cardiac complications (Cottin and Brown (2019) RESPIR. RES. 20(1): 13; Winstone et al. (2014) CHEST 146(2): 422-436). Mortality is highest during the first 5 years, when disease progression is most rapid, with cumulative survival rates of 70% and 50% at 5 and 10 years, respectively. Esophageal symptoms are the second most common reported SSc finding (after skin) with 50% to 90% of patients reporting symptoms of dysphagia, heartburn, or gastroesophageal reflux disease (Abu-Shakra et al. (1994) ARTHRITIS RHEUM. 24(1): 29-39; Sjogren (1994) ARTHRITIS RHEUM. 37(9): 1265-1282). Furthermore, some patients show objective evidence of esophageal dysmotility by functional luminal imaging probe (FLIP) panometry, which provides a comprehensive evaluation of esophageal function, distensibility, and contractility at the time of endoscopy (Carlson et al. (2015) CURR. RHEUMATOL. REP. 17(1): 475).

Currently, there is no cure for SSc, and there are no US Food and Drug Administration (FDA)-approved medicinal products for the treatment of SSc. Current treatment options are primarily limited to immunosuppressants and targeted biologic agents, which are used to minimize disease impact and development of major complications (Kowal-Bielecka et al. (2017) ANN. RHEUM. DIS. 76(8): 1327-1339). These therapies offer only modest benefit in delaying disease progression, and the prognosis for patients with SSc, especially dcSSc, remains poor (Elhai et al. (2012) RHEUMATOLOGY (OXFORD) 51(6): 1017-1026; Sullivan et al. (2018) N. ENGL. J. MED. 378(1): 35-47). Current therapy is used to control symptoms, prevent immediate clinical consequences, reduce organ damage, and decrease long-term morbidity and mortality.

### Investigational Product

The investigational product, CAB-001, is an autologous CD19-targeting chimeric antigen receptor (CAR) T cell. The CAR comprises a fully human scFv which specifically binds CD19, a CD8α transmembrane domain, a 4-1BB costimulatory domain, and a CD3ζ signaling domain. The anti-CD19 scFv comprises the amino acid sequence of SEQ ID NO: 9, and the construct comprises the amino acid sequence of SEQ ID NO: 23 (or SEQ ID NO: 27, including a CD8α signal peptide).

### Objectives and Endpoints

A primary objective of this study is to evaluate the safety and tolerability of CAB-001 in subjects with systemic sclerosis at risk for organ disease progression, measured by incidence of adverse events (AEs) occurring within 28 days after CAB-001 infusion.

An additional objective of this study is to evaluate the safety and tolerability of CAB-001 in subjects with systemic sclerosis over 156 weeks, measured by AEs, vital signs, physical exam findings, and clinical laboratory test results occurring within 156 weeks after CAB-001 infusion.

An additional objective of this study is to evaluate the effects of CAB-001 on hematologic and immunologic parameters including WBCs and T, B, NK, and myeloid populations, measured as changes from baseline in WBC with differential, as well as T, B, NK cell counts and immunophenotypic sub-populations after CAB-001 infusion.

An additional objective of this study is to evaluate CAB-001 persistence and kinetics after infusion, measured by the number and percentage of CAB-001 positive cells in peripheral blood of participants overtime.

An additional objective of this study is to evaluate the effects of CAB-001 on SSc disease activity, measured as: (1) proportion of subjects achieving revised CRISS criteria at 12, 24, 52, 80, 104, and 156 weeks after CAB-001 infusion; (2) proportion of subjects achieving revised CRISS criteria with no immunosuppressive therapy at 12, 24, 36, 52, 80, 104 and 156 weeks after CAB-001 infusion; (3) proportion of subjects achieving response to each individual element of the revised CRISS, including (a) no significant SSc-related event as defined by the revised CRISS, (b) ≥5% improvement in the percent predicted FVC compared to baseline, (c) ≥25% decrease in mRSS, HAQ-DI, PtGA, and PGA compared to baseline; (4) change in pulmonary function as measured by the percent of predicted FVC and adjusted DLCO at 12, 24, 52, 80, 104, 130, and 156 weeks after CAB-001 infusion; (5) change in HRCT quantification at 12, 24, 52, and 104 weeks after CAB-001 infusion; (6) change in mRSS score; (7) Change in serum CRP and ESR; (8) proportion of subjects developing new or worsening end organ dysfunction, including pulmonary, cardiac, and renal disease; and (9) Change in SSc-associated antibodies.

An additional objective of this study is to evaluate the effect of CAB-001 on concomitant immunosuppressive therapy use and other SSc-related therapy, measured as the proportion of subjects who require no immunosuppressive therapy after CAB-001 infusion.

An additional objective of this study is to evaluate the effect of CAB-001 on overall and event-free survival, measured as overall survival and as event-free survival (defined as survival without significant organ damage).

An additional objective of this study is to evaluate the effect of CAB-001 on patient reported outcomes, measured using SHAQ, SSPRO, UCLA SCTC GIT, PtGA, FACIT-F, Pain NRS, and/or QOL (SF-36 and EQ-5D-5L).

### Study Design

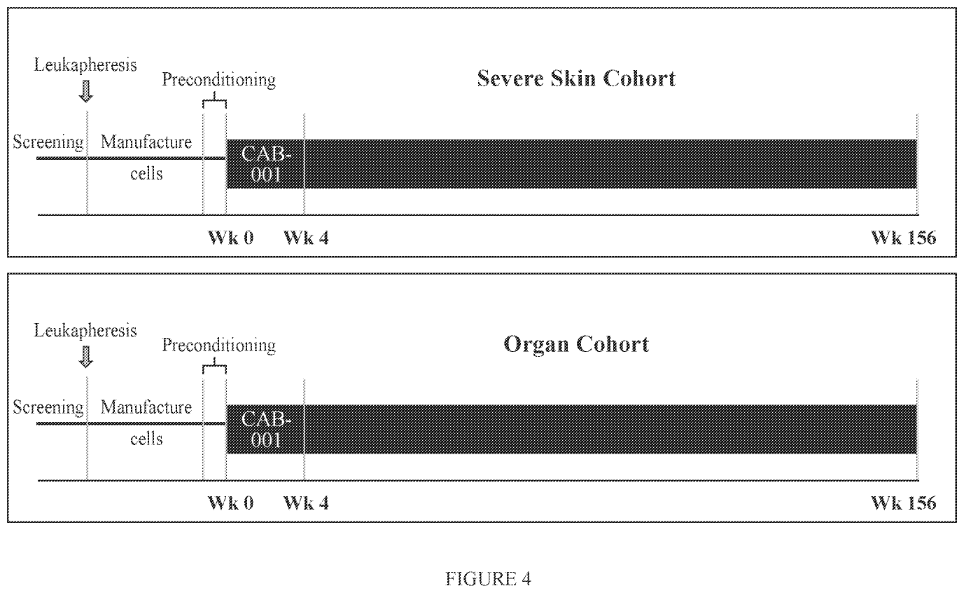

The design of this study is summarized in FIG. 4. The study includes two cohorts based upon the prominent feature(s) of their SSc phenotype:

Severe Skin Cohort: Subjects only meet criteria for skin involvement and do not meet pulmonary, cardiac, or renal involvement criteria as defined in Inclusion Criterion (6).

Organ Cohort: Subjects meet pulmonary, cardiac, or renal involvement criteria as defined in Inclusion Criterion (6) regardless of skin involvement.

Each participant undergoes leukapheresis, and the CAB-001 cell product for the participant is manufactured. Briefly, autologous T cells are genetically modified ex vivo by transduction with a lentivirus vector. Upon completion of the manufacturing process, the drug product is formulated and cryopreserved.

Following leukapheresis and cell manufacturing, participants are administered a preconditioning regimen. The preconditioning regimen may be a standard preconditioning regimen (e.g., IV Fludarabine administered at 25 mg/m² on Days −5, −4 and −3, and IV Cyclophosphamide administered at 1,000 mg/m² on Day −3) or may be a preconditioning regimen comprising a reduced dose of cyclophosphamide and/or fludarabine (e.g., as described in TABLE 1). CAB-001 is administered via IV infusion to participants at one of the four following doses: 1×10⁶ cells/kg, 3×10⁵ cells/kg, 3×10⁶ cells/kg, 1×10⁷ cells/kg.

Participants are evaluated for toxicities related to the CAB-001 therapy and its effect on their underlying disease during an initial 28-day assessment period. Participants are monitored in the days post-infusion, and subsequent visits occur approximately on Days 5, 8, 15, 22, and 29 post-infusion. In addition to routine chemistry and hematology tests, lab tests for PK sampling, coagulation panel, CRS labs, serum cytokine assessment, and CAR T assessment are performed through Day 29. Skin punch biopsies are also collected at the Day 22 visit. After the Day 29 post-infusion evaluation, participants undergo evaluations every 4 weeks (±7 days) in the first year, approximately every 12 weeks (+14 days) in the second year, and approximately once every 26 weeks (±30 days) in the third year. All participants are evaluated for safety, tolerability, efficacy, and quality of life for 156 weeks following infusion. Participants may also monitored for 15 years after treatment, as appropriate.

### Inclusion and Exclusion Criteria

Inclusion criteria for participation in this study include: (1) Able to provide informed consent; (2) be 18 or older and 70 or younger; (3) diagnosis of limited or diffuse SSc as defined by the 2013 American College of Rheumatology and European League Against Rheumatism classification criteria; (4) disease duration ≤7 years from first non-Raynaud's phenomenon symptom; (5) inadequate response or intolerance to at least two immunomodulatory medications in the past to treat SSc (subjects must have been treated with at least two of the following medications for a duration of at least 12 weeks to be eligible: azathioprine, corticosteroids, CY, hydroxychloroquine, IVIg, Janus kinase (JAK) inhibitors, mycophenolate mofetil or mycophenolic acid, methotrexate, rituximab, and/or tocilizumab); (6) documented evidence of significant skin, pulmonary, renal, or cardiac involvement defined as meeting ≥1 of the following conditions: I. Severe Skin Cohort subjects must meet the criteria for skin involvement only, which is all of (a) modified Rodnan Skin Score (mRSS) score >15 and <40, Health Assessment Questionnaire Disability Index (HAQ-DI) of >1.0 or elevated erythrocyte sedimentation rate or C reactive protein (CRP), and (c) disease duration is less than 3 years and is rapidly progressive, defined as a 5-unit increase in mRSS over a 12-month period; II. Organ Cohort subjects must meet the criteria for either renal (IIa), cardiac (IIb), or pulmonary (IIc) involvement and may or may not meet the skin involvement criteria: IIa. renal involvement criteria include a history of renal crisis that is not active at time of screening, wherein serum creatinine is stable (with no increases ≥20%) for a minimum of 24 weeks post-renal crisis at the time of the screening visit; IIb. cardiac involvement criteria are that the condition is clinically stable with or without medication, and non-scleroderma related cardiac disorders must have been reasonably excluded (≥1 of the following: (a) New York Heart Association (NYHA) Class I or II heart disease which is well-controlled (b) atrial or ventricular arrhythmias; (c) 2nd or 3rd degree atrioventricular block; and (d) myocarditis); IIIc. pulmonary involvement criteria are that the subject has either (i) or (ii): (i) Forced vital capacity (FVC) >55% and <80% predicted or hemoglobin-adjusted diffusing capacity of the lung for carbon monoxide (DLCO) >45% and <80% predicted and ILD by high-resolution computed tomography of the chest, or (ii) rapidly progressive fibrosing ILD as defined by 2 of the 3 criteria occurring within the past year and with no alternative explanation: (sub-criterion 1) worsening respiratory symptoms, (sub-criterion 2) physiological evidence of disease progression by either an absolute decline in FVC≥5% predicted within 1 year of follow-up or an absolute decline in DLCO adjusted for hemoglobin ≥10% predicted within 1 year of follow-up, and (sub-criterion 3) radiological evidence of disease progression as indicated by one or more of the following: i. increased extent or severity of traction bronchiectasis and bronchiolectasis, ii. new ground-glass opacity with traction bronchiectasis, iii. new fine reticulation, iv. increased extent or increased coarseness of reticular abnormality, v. new or increased honeycombing, and vi. increased lobar volume loss; (7) the subject is an appropriate candidate for the study, in the opinion of an investigator who has considered the potential benefits of other available therapies; (8) have received all recommended vaccinations per Centers for Disease Control and Prevention (CDC) or institutional guidelines for immunocompromised individuals before or during screening (live vaccines must be administered at least 30 days prior to the pre-infusion visit, and non-live vaccines should be administered to subjects at least 2 weeks prior to the start of study drug infusion (and, if possible, non-live vaccines should also be administered at least 2 weeks prior to leukapheresis); (9) clinical stability based upon the judgement of investigator; (10) if subject is currently receiving standard immunosuppressive therapy, the therapy must have been initiated at least 12 weeks prior to screening and on a stable dose for at least 8 weeks prior to the screening visit, except for dose reduction due to safety or tolerability; (11) women of reproductive potential must agree to use 2 acceptable methods of birth control from Screening to a minimum of 52 weeks after the CAB-001 infusion; (12) sexually active men, both reproductively competent and vasectomized, who are to receive CAB-001 are required to use condoms with spermicide from screening until at least 52 weeks after CAB-001 infusion, and males of reproductive potential must also agree not to donate sperm from Screening to at least 52 weeks after the CAB-001 infusion.

Exclusion criteria include patients you have any of the following criteria: (1) systemic sclerosis-like illness or a primary diagnosis of rheumatic AD other than SSc, including but not limited to rheumatoid arthritis, SLE, polymyositis, dermatomyositis, systemic vasculitis, Sjögren's syndrome, anti-synthetase syndrome, or mixed connective tissue disease as determined by investigator (Secondary Sjögren's or scleroderma myopathy is allowed); (2) 2. Significant pulmonary dysfunction, defined as any of a.-c.: a. resting oxygen saturation <92% without supplemental oxygen, b. FVC≤555% predicted, and c. DLCO adjusted for hemoglobin of ≤45% predicted; (3) significant PAH, defined as mean pulmonary artery pressure ≥25 mmHg at rest on right heart catheterization (RHC) (subjects who have had a RHC demonstrating significant PAH within 12 months of Screening are ineligible; a screening echocardiogram will be performed on all other subjects, and a RHC will be required if any of the following echocardiographic findings suggestive of PAH are identified: i. peak tricuspid regurgitation velocity >2.8 m/see, ii. elevated right ventricular systolic pressure, iii. abnormality of right atrial size, shape, or wall thickness, iv. abnormality of right ventricular size, shape, or wall thickness, and v. abnormal septal wall shape); (4) significant cardiac disease, including any of the following: a. unstable angina or myocardial infarction or coronary artery bypass graft within 24 weeks prior to leukapheresis, uncontrolled clinically significant arrhythmia, c. clinical evidence of CHF NYHA Class III or IV, d. moderate to severe left ventricular dysfunction (left ventricular ejection fraction <45% on Screening echocardiogram), or e. prior insertion of a pacemaker or cardioverter-defibrillator; (5) significant renal disease, including any of the following: a. estimated glomerular filtration rate <60 mL/min/1.73 m2 using the Chronic Kidney Disease-Epidemiology Collaboration formula, b. Scleroderma renal crisis in the last 24 weeks, or c. glomerulonephritis or overlap syndromes; (6) clinical evidence for active aspiration as determined by investigator; (7) alanine aminotransferase, aspartate aminotransferase, or bilirubin >2 times the upper limit of normal; (8) documented history of seizures, encephalopathy, or cerebral edema; (9) active gastric antral vascular ectasia (GAVE, “watermelon stomach”) at Screening esophagogastroduodenoscopy (EGD) (subjects who have a documented negative EGD within 24 weeks of Screening or subjects who have successfully been treated for GAVE, as documented by post-treatment EGD within 24 weeks, are eligible); (10) hematologic abnormality as defined in a.-d.: a. absolute lymphocyte count <500 cells/μL, b. absolute neutrophil count <1,000 cells/μL, c. platelet count <80,000 cells/μL d. hemoglobin <8.0 g/dL; (11) subjects who have been given any of a.-i.: a. prednisone at dose of >10 mg/day within 2 weeks prior to screening, b. prednisone 1 mg/kg/day or its equivalent for concurrent illness such as asthma for ≥5 days on >2 occasions during the previous 24 weeks, or c. Rituximab or other B cell-depleting agents within 24 weeks prior to screening, or within 12 weeks if there are laboratory results indicating presence of CD19+ B cells, d. Abatacept within 8 weeks of screening, e. IL-6 inhibiting drugs (e.g., tocilizumab) within 12 weeks of screening, f. TNF inhibitors within 12 weeks of screening, g. Leflunomide within 8 weeks of screening, h. therapeutic pheresis within 4 weeks of screening, i. any investigational agent within 4 weeks or 5 half-lives, whichever is longer; (12) known malignancy or a history of malignancy within 5 years, with exception of excised/cured local basal or squamous cell carcinoma of the skin or carcinoma in situ of the uterine cervix; (13) planned major surgery during the study evaluation period; (14) Contraindications to leukapheresis; (15) history of anaphylactic or severe systemic reaction to FLU, CY, or any of their metabolites; (16) positive test for human immunodeficiency virus, hepatitis C antibody, hepatitis B surface antigen, or evidence of active or chronic tuberculosis at screening; (17) active infection requiring medical intervention at screening; (18) any inflammatory autoimmune disorder other than SSc requiring immunosuppressive therapies; (19) current symptoms of severe, progressive, or uncontrolled renal, hepatic, hematological, gastrointestinal, pulmonary, psychiatric, cardiac, neurological, or cerebral disease including severe and uncontrolled infection such as sepsis and opportunistic infections; (20) previous CAR T cell therapy; (21) prior solid organ (heart, liver, kidney, lung) transplant or hematopoietic stem cell transplant; (22) pregnant or lactating women; (23) unable or unwilling to comply with the protocol.

It is contemplated that the CD19-CAR T cell therapy will be effective to treat systemic sclerosis. It is further contemplated that the use of the reduced preconditioning regimen prior to treatment with CD19-CAR T cell therapy will be effective in the treatment of systemic sclerosis, and that a standard, full-dose preconditioning regimen is not required.

### Example 8

This example describes a Phase 1/2, open-label study designed to evaluate the safety and efficacy of CAB-001 in participants with active idiopathic inflammatory myopathies (IIMs, or myositis).

### Background

IIMs (myositis) are a heterogenous group of rare acquired, systemic autoimmune diseases characterized by inflammation and muscle weakness. The symptoms of IIMs can vary widely and may include muscle weakness, fatigue, dysphagia, and dyspnea. In some cases, IIMs may also affect other organs and systems in the body, such as the lungs, heart, or skin. Due to pain and weakness, patients with IIM may have increasing difficulty with activities requiring the use of proximal muscles, such as rising from a chair, climbing stairs, or lifting objects (Malik et al. (2016) FRONT. NEUROL. 7: 64). Extra-muscular disease activity may include fevers, arthralgia, arrythmias, or cardiac dysfunction, as well as pulmonary complications, frequently due to interstitial lung disease (ILD) (Lundberg et al. (2021) NAT. REV. DIS. PRIMERS 7(1): 86).

Several subtypes of IIM, which are unified by inflammation and proximal muscle weakness, include dermatomyositis (DM), anti-synthetase syndrome (ASyS), and immune-mediated necrotizing myopathy (IMNM). These IIM subtypes typically affect middle-aged individuals, particularly women. DM, the most common subtype with about 34,000 prevalent US patients (Kronzer et al. (2023) ARTHRITIS CARE RES. (HOBOKEN) 75(2): 348-355), involves a characteristic rash in addition to muscle weakness. Anti-synthetase syndrome affects about 10,000 patients in the US and is characterized by autoantibodies against aminoacyl-transfer ribonucleic acid (tRNA) synthetases, which cause inflammation and muscle weakness in addition to systemic symptoms such as ILD, which is observed in 65%-100% of patients (Iaccarino et al. (2014) J. AUTOIMMUN. 48-49: 122-127). IMNM, which affects about 6,000 patients in the US, is characterized by muscle tissue death and weakness (Allenbach et al. (2020) NAT. REV. RHEUMATOL. 16(12): 689-701). All three subtypes can lead to severe functional impairment and may be life-threatening.

There is currently no cure for IIM, and available treatment options have limited effectiveness in IIM patients, with potential for serious long-term side effects. Treatment for DM, ASyS, and IMNM typically involves medications to suppress the immune system and reduce inflammation. Although some patients may have only mild symptoms that can be managed with medications, a significant portion of the IIM patient population has disease that requires more aggressive treatment or that remains refractory to existing medications (Moghadam-Kia and Oddis (2022) CURR. OPIN. PHARMACOL. 65: 102257).

### Investigational Product

The investigational product, CAB-001, is an autologous CD19-targeting chimeric antigen receptor (CAR) T cell. The CAR comprises a fully human scFv which specifically binds CD19, a CD8α transmembrane domain, a 4-1BB costimulatory domain, and a CD3ζ signaling domain. The anti-CD19 scFv comprises the amino acid sequence of SEQ ID NO: 9, and the construct comprises the amino acid sequence of SEQ ID NO: 23 (or SEQ ID NO: 27, including a CD8α signal peptide).

### Objectives and Endpoints

A primary objective of this study is to evaluate the safety and tolerability of CAB-001 in subjects with active IIM over 28 days, measured by incidence of adverse events (AEs) occurring within 28 days after CAB-001 infusion.

An additional objective of this study is to evaluate the safety and tolerability of CAB-001 in subjects with active IIM over 156 weeks, measured by AEs, vital signs, physical exam findings, and clinical laboratory test results occurring within 156 weeks after CAB-001 infusion.

An additional objective of this study is to evaluate the effects of CAB-001 on WBCs, including B cell counts, measured as changes from baseline in WBC with differential after CAB-001 infusion, including B cell counts.

An additional objective of this study is to evaluate CAB-001 persistence and kinetics after infusion, measured by the number and percentage of CAB-001 positive cells in peripheral blood of participants overtime.

An additional objective of this study is to evaluate the effects of CAB-001 on IIM serology, measured using changes in muscle enzymes (CK, LDH, AST, ALT, or aldolase), and using changes in myositis-specific antibody (MSA) levels.

An additional objective of this study is to evaluate the effects of CAB-001 on IIM disease activity, measured using: (1) change from baseline in the TIS; (2) proportion of subjects achieving major (TIS≥60), moderate (TIS≥40), and minimal (TIS≥20) response; (3) change in individual 6 core set measures (CSMs); (4) change in the Sit-to-Stand Test results; (5) change in Joint Counts; (6) time to minimal, moderate, and major response; (7) proportion of subjects with maintenance of TIS response; (8) change in IIM-related rashes or lesions on CDASI; (9) muscle changes as demonstrated on MRI evaluation; (10) change in PFTs; (11) change in HRCT chest assessments.

An additional objective of this study is to evaluate the effect of CAB-001 on concomitant steroid use and other IIM-related therapy, measured as change from baseline in the dose of concomitant corticosteroids and other IIM-related therapies, as the proportion of subjects achieving a dose of ≤5 mg/day oral prednisone or equivalent, and as the proportion of subjects who require no IIM-related therapies.

An additional objective of this study is to evaluate the effect of CAB-001 on patient reported outcomes and health-related quality of life, measured as changes from baseline in FACIT-F, pain NRS, PROMIS physical function 20a v2.0, PGIC, SF-36v2, and EQ-5D-5L scores.

An additional objective of this study is to evaluate the effect of CAB-001 on IIM disease flares, measured as the proportion of subjects who meet confirmed deterioration criteria, and as the time to confirmed deterioration.

An additional objective of this study is to evaluate the effect of CAB-001 on drug-free response, measured as the proportions of subjects achieving drug free major, moderate, and minimal responses, and also measured as time to achievement of drug-free major, moderate, and minimal responses.

### Study Design

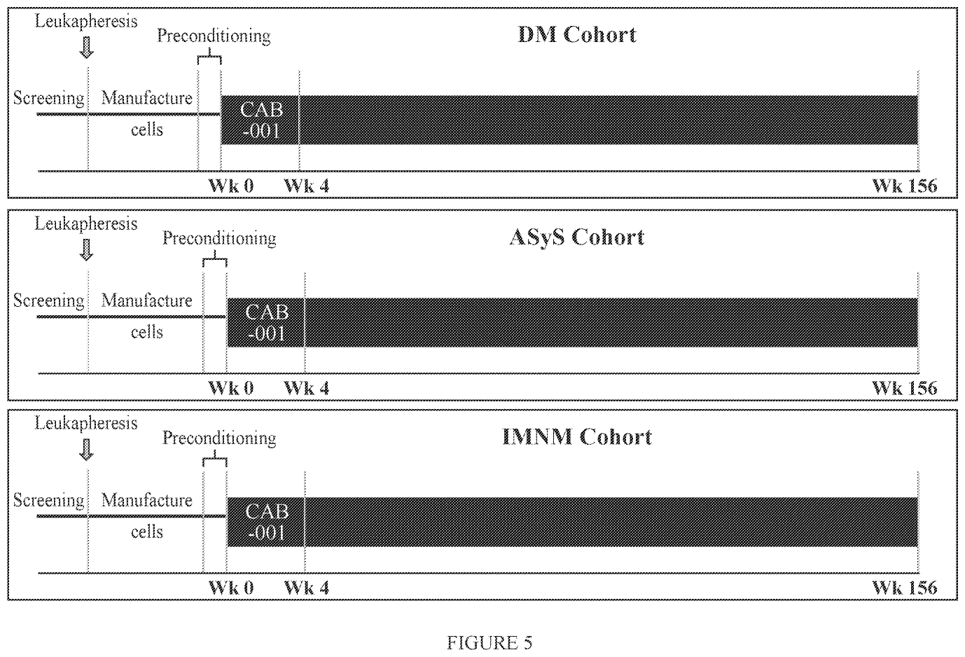

The design of this study is summarized in FIG. 5. The study includes three separate cohorts: a DM cohort, an IMNM cohort, and an ASyS cohort. Each participant in each cohort undergoes leukapheresis, and the CAB-001 cell product for the participant is manufactured. Briefly, autologous T cells are genetically modified ex vivo by transduction with a lentivirus vector. Upon completion of the manufacturing process, the drug product is formulated and cryopreserved.

Following leukapheresis and cell manufacturing, participants are administered a preconditioning regimen. The preconditioning regimen may be a standard preconditioning regimen (e.g., IV Fludarabine administered at 25 mg/m² on Days −5, −4 and −3, and IV Cyclophosphamide administered at 1,000 mg/m² on Day −3) or may be a preconditioning regimen comprising a reduced dose of cyclophosphamide and/or fludarabine (e.g., as described in TABLE 1). CAB-001 is administered via IV infusion to participants at one of the four following doses: 1×10⁶ cells/kg, 3×10⁵ cells/kg, 3×10⁶ cells/kg, 1×10⁷ cells/kg.

Participants are evaluated for toxicities related to the CAB-001 therapy and its effect on their underlying disease during an initial 28-day assessment period. Participants are monitored post-infusion, and subsequent visits occur approximately on Days 2, 5, 8, 15, 22, and 29 post-infusion. In addition to routine chemistry and hematology tests, lab tests for PK sampling, coagulation panel, CRS labs, serum cytokine assessment, complement research assessment, and CAR T assessment are performed through Day 29. After the Day 29 post-infusion evaluation, participants undergo evaluations every 4 weeks (±7 days) in the first year, approximately every 12 weeks (±14 days) in the second year, and approximately once every 26 weeks (±30 days) in the third year. All participants are evaluated for safety, tolerability, efficacy, and quality of life for 156 weeks following infusion. Participants may also monitored for 15 years after treatment, as appropriate

### Inclusion and Exclusion Criteria

Inclusion criteria for participation in this study include: (1) able to provide informed consent; (2) be 18 or older and 70 or younger; (3) Eastern Cooperative Oncology Group performance status 0-1; (4) a definite or probable clinical diagnosis of IIM, based on the 2017 The European League Against Rheumatism/American College of Rheumatology classification criteria for IIM (adults who were diagnosed with juvenile IIM are eligible); (5) a diagnosis of DM, ASyS, or IMNM, based on the presence of serum MSAs prior to or during screening (historical autoantibody results may be used for eligibility verification and cohort assignment, if available; autoantibody testing will be performed on all subjects at screening); (6) evidence of active disease as defined by the presence of one of the following criteria: a. Creatine kinase (CK) ≥2 times the upper limit of normal (ULN) at screening, b. muscle biopsy, magnetic resonance imaging (MRI), or electromyography with evidence of active disease within 26 weeks prior to or during screening, or c. DM rash with a Cutaneous Dermatomyositis Disease Area and Severity Index-Activity (CDASI-A) ≥6; (7) muscle weakness as defined by a Manual Muscle Test-8 score ≤142; (8) minimal core set measure (CSM) requirement as defined by the presence of ≥2 of the following a.-e.: a. CK, lactate dehydrogenase (LDH), aspartate aminotransaminase (AST), or alanine aminotransferase (ALT) ≥2 times the ULN, b. Visual Analogue Scale (VAS) of patient global disease activity ≥3 cm, c. VAS of physician's global disease activity ≥3 cm, d. VAS of extra-muscular global disease activity as assessed by Myositis Disease Activity Assessment Tool (MDAAT) ≥2 cm, or e. Health Assessment Questionnaire—Disability Index (HAQ-DI) ≥0.375; (9) inadequately managed IIM to and/or intolerant to at least 3 standard immunosuppressive therapies (see Appendix 2) during a period of up to 3 years prior to Screening, either in combination or as monotherapy; (10a) if a subject is currently receiving standard immunosuppressive therapy other than IVIg, subcutaneous immunoglobulin (SCIg), plasma exchange, or rituximab, the therapy must have been initiated at least 12 weeks prior to screening and on a stable dose for at least 8 weeks prior to screening, except for dose reduction due to safety or tolerability; (10b) if a subject is currently receiving an oral corticosteroid, the subject must be receiving ≤20 mg prednisone or equivalent and on a stable dose for at least 2 weeks prior to screening; if a subject is currently receiving IVIg or SCIg, the therapy must have been initiated at least 12 weeks prior to screening; (11) the subject is an appropriate candidate for the study, in the opinion of investigator; (12) have received all recommended vaccinations per the Centers for Disease Control and Prevention (CDC) or local/institutional guidelines for immune compromised individuals (live vaccines must be administered at least 30 days prior to pre-infusion visit, and non-live vaccines should be administered to subjects at least 2 weeks prior to the start of CAB-001 infusion and, if possible, non-live vaccines should also be administered at least 2 weeks prior to leukapheresis); (13) estimated glomerular filtration rate of ≥60 mL/min/1.73 m2 using the chronic kidney disease-epidemiology equation; (14) clinically stable by vital signs assessments at screening including: a. Systolic blood pressure ≥90 mmHg and ≤170 mmHg, b. diastolic blood pressure ≥55 mmHg and ≤10⁵ mmHg, c. pulse ≥50 beats per minute and ≤110 beats per minute, d. Respiratory rate ≥12 and ≤20 breaths per minute, and e. afebrile; (15) women of reproductive potential must agree to use 2 acceptable methods of birth control from screening to a minimum of 52 weeks after the CAB-001 infusion; (16) sexually active men, both reproductively competent and vasectomized, who are scheduled to receive CAB-001 are required to use condoms with spermicide from Screening until at least 52 weeks after CAB-001 infusion, and males of reproductive potential must also agree not to donate sperm from Screening to at least 52 weeks after the CAB-001 infusion.

Exclusion criteria include patients you have any of the following criteria: (la) cancer-associated myositis, defined as the diagnosis of myositis within 2 years of the diagnosis of cancer except basal or squamous cell skin cancer or carcinoma in situ of the cervix that has been excised and cured and at least 1 year since excision (1b) evidence of active malignant disease, malignancies diagnosed within the previous 5 years (including hematological malignancies and solid tumors); (2) lung impairment meeting criteria of a through c: a. resting oxygen saturation <93% without supplemental oxygen and b. forced vital capacity (FVC)<60%, c. in subjects who have had pulmonary function tests, spirometry results of forced expiratory volume (FEV1)/FVC<0.7 or FEV1<80% predicted after bronchodilators, or diffusing capacity of the lungs for carbon monoxide results <60% predicted; (3) impaired cardiac function or clinically significant cardiac disease including: a. unstable angina or myocardial infarction or coronary artery bypass graft within 26 weeks prior to leukapheresis, b. New York Heart Association stage III or IV congestive heart failure, c. history of clinically significant cardiac arrhythmia (e.g., ventricular tachycardia), complete left bundle branch block, or high-grade atrioventricular block, d. history of severe nonischemic cardiomyopathy, or e. left ventricular ejection fraction <45%, as assessed by echocardiogram or multi-gated acquisition scan (if performed)≤8 weeks of leukapheresis; (4) other inflammatory and noninflammatory myopathies, including glucocorticoid-induced myopathy, inclusion body myositis, metabolic myopathies, muscle dystrophies or a family history of muscle dystrophy, and drug-induced or endocrine induced myositis, except for secondary Sjögren's syndrome with overlap myositis; (5) planned initiation of a muscle strengthening regimen during the study evaluation period; (6) planned major surgery (including joint surgery) during the study evaluation period; (7) treatment with rituximab or other B cell-depleting agent within 26 weeks prior to screening, or within 12 weeks if there are laboratory results indicating presence of CD19+ B cells; (8) any other non-anti-tumor necrosis factor (anti-TNF) biologic treatment (e.g., abatacept, tocilizumab) in the 12 weeks prior to screening; (9) contraindication to leukapheresis; (10) history of anaphylactic or severe systemic reaction to FLU, CY, or any of their metabolites; (11) any of the following clinical laboratory values a through d at screening: a. absolute lymphocyte count <500/μL, b. absolute neutrophil count <1,000/μL, c. hemoglobin <8 g/dL, or d. platelet count <80,000/μL; (12) positive test for human immunodeficiency virus (HIV), hepatitis C antibody, hepatitis B surface antigen, or evidence of active or chronic tuberculosis at screening; (13) active infection requiring medical intervention at screening; (14) active, inflammatory autoimmune disorder other than IIM requiring immunosuppressive therapies; (15) current symptoms of severe, progressive, or uncontrolled renal, hepatic, hematological, gastrointestinal, pulmonary, psychiatric, cardiac, neurological, or cerebral disease, including severe and uncontrolled infections, such as sepsis and opportunistic infections; (16) previous CAR T cell therapy; (17) prior solid organ (heart, liver, kidney, lung) transplant or hematopoietic cell transplant; (18) pregnant or lactating, or plan to become pregnant during the 52 weeks following the CAB-001 infusion; (19) men of reproductive potential who plan to father a child in the 52 weeks following CAB-001 infusion; (20) unable or unwilling to comply with protocol; (21) treatment with any investigational agent within 4 weeks or 5 half-lives, whichever is longer (subjects who had received a C5 inhibitor (e.g., eculizumab, ravulizumab) may be eligible earlier than 5 half-lives after the last C5 inhibitor administration if, at screening, they have evidence of complement lab testing (i.e., total hemolytic complement measurement) that has normalized or returned to pre-treatment levels.)

It is contemplated that the CD19-CAR T cell therapy will be effective to treat myositis (IIMs, e.g., DM, IMNM, or ASyS). It is further contemplated that the use of the reduced preconditioning regimen prior to treatment with CD19-CAR T cell therapy will be effective in the treatment of myositis, and that a standard, full-dose preconditioning regimen is not required.

### Example 9

This example describes a phase I/II, open-label study to investigate the safety and efficacy of autologous CD19-specific CAR T cells (CAB-001) in subjects with active systemic lupus erythematosus.

### Background

SLE is a chronic autoimmune disorder characterized by autoantibody production and abnormal B cell function. SLE presents with fluctuating severity and may cause tissue damage in a variety of organs over time. Lupus nephritis (LN) is a common severe manifestation of SLE, which can lead to significant morbidity and mortality. There is no approved cure for SLE, and available treatment options have limited effectiveness in SLE patients with serious long-term side effects.

### Investigational Product

The investigational product, CAB-001, is an autologous CD19-targeting chimeric antigen receptor (CAR) T cell. The CAR comprises a fully human scFv which specifically binds CD19, a CD8α transmembrane domain, a 4-1BB costimulatory domain, and a CD3ζ signaling domain. The anti-CD19 scFv comprises the amino acid sequence of SEQ ID NO: 9, and the construct comprises the amino acid sequence of SEQ ID NO: 23 (or SEQ ID NO: 27, including a CD8α signal peptide).

### Objectives and Endpoints

A primary objective of this study is to evaluate the safety and tolerability of CAB-001 in subjects with active SLE over 28 days, measured by incidence of adverse events (AEs) occurring within 28 days after CAB-001 infusion, including dose-limiting toxicities (DLTs) and AEs related to CAB-001.

An additional objective of this study is to evaluate the safety and tolerability of CAB-001 in subjects with active SLE over 156 weeks, measured by AEs, vital signs, physical examination, and clinical laboratory tests occurring within 156 weeks after CAB-001 infusion.

An additional objective of this study is to evaluate the effect of CAB-001 regimen on white blood cell counts, including B cell counts, measured from baseline in white blood cell counts with differential, including B cell counts.

An additional objective of this study is to evaluate CAB-001 expansion and persistence in vivo, measured as the number and percentage of CAB-001-positive cells in the peripheral blood of subjects over time.

An additional objective of this study is to evaluate the effect of CAB-001 on SLE serology, measured by the changes from baseline in anti-double stranded DNA (dsDNA) antibodies, complement component 3 (C3), complement component 4 (C4), and 50% hemolytic complement (CH50). Autoantibodies including ANA, anticardiolipin, anti-beta-2 glycoprotein 1, anti-Sm, anti-Ro, anti-La, anti-C1Q and anti-RNP may also be analyzed.

An additional objective of this study is to evaluate the effect of CAB-001 on SLE disease activity, measured as: the changes in urine protein:creatinine ratio (UPCR), estimated glomerular filtration rate (eGFR), SLEDAI-2K score, British Isles Lupus Assessment Group (BILAG)-2004 score, Physician Global Assessment (PGA) score, joint counts, Cutaneous Lupus Erythematosus Disease Area and Severity Index (CLASI) score, the SLICC Damage Index (SDI); and as the proportions of subjects achieving complete renal response (CRR; for LN cohort only), SRI-4, SRI-5, SRI-6, BICLA responses, LLDAS and DORIS remission criteria.

An additional objective of this study is to evaluate the time to achieve disease response after CAB-001 infusion, measured as the time to achieve complete renal response (for LN cohort only), SRI-4, SRI-5, SRI-6, BICLA responses, LLDAS, and DORIS remission.

An additional objective of this study is to evaluate the effect of CAB-001 on concomitant corticosteroid use and other SLE-related therapy, measured by the change from baseline in the dose of concomitant corticosteroids and other SLE-related therapies, proportion of subjects achieving a dose ≤5 mg/day of oral prednisone (or equivalent), and proportion of subjects who require no SLE-related therapies.

An additional objective of this study is to evaluate the effect of CAB-001 on patient-reported outcomes and health-related quality of life, measured as changes from baseline in 36-Item Short Form Survey Version 2 (SF-36v2), Pain Numeric Rating Scale (NRS), Functional Assessment of Chronic Illness Therapy-Fatigue (FACIT-F), Patient Global Assessment (PtGA), Lupus Quality of Life Scale (Lupus QoL), and EQ-5D-5L scores.

An additional objective of this study is to evaluate the effect of CAB-001 on disease flares, measured as the incidences of mild/moderate and severe disease flares, as defined per BILAG-2004 and modified SELENA-SLEDAI Flare Index (SFI), and as the time to mild/moderate and severe flare.

An additional objective of this study is to evaluate the effect of CAB-001 on drug-free response, measured as proportions of subjects achieving drug free complete renal response (for LN cohort only), SRI-4, SRI-5, SRI-6, BICLA responses, LLDAS, and DORIS remission.

An additional objective of this study is to evaluate the time to achieve drug-free response after CAB-001 infusion, measured as time to achievement of drug-free complete renal response (for LN cohort only), SRI-4, SRI-5, SRI-6, BICLA responses, LLDAS, and DORIS remission.

### Study Design

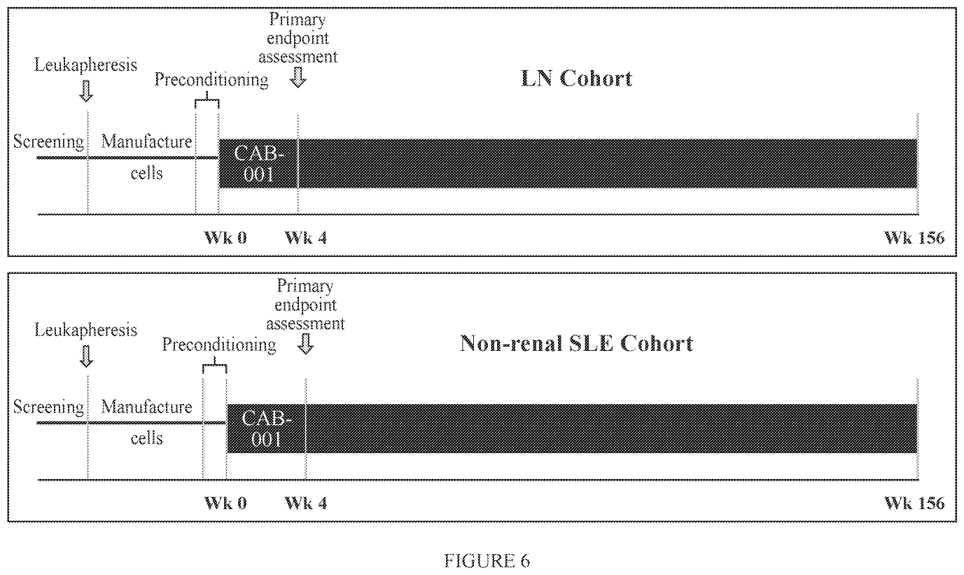

The design of this study is summarized in FIG. 6. The study includes two separate cohorts: a cohort of subjects with lupus nephritis (LN; “the LN Cohort”) and a cohort of subjects who do not fulfill the criteria for LN (“the non-renal SLE Cohort”). Each participant in each cohort undergoes leukapheresis, and the CAB-001 cell product for the participant is manufactured. Briefly, autologous T cells are genetically modified ex vivo by transduction with a lentivirus vector. Upon completion of the manufacturing process, the drug product is formulated and cryopreserved.

Following leukapheresis and cell manufacturing, participants are administered a preconditioning regimen. The preconditioning regimen may be a standard preconditioning regimen (e.g., IV Fludarabine administered at 25 mg/m² on Days −5, −4 and −3, and IV Cyclophosphamide administered at 1,000 mg/m² on Day −3) or may be a preconditioning regimen comprising a reduced dose of cyclophosphamide and/or fludarabine (e.g., as described in TABLE 1). CAB-001 is administered via IV infusion to participants at one of the four following doses: 1×10⁶ cells/kg, 3×10⁵ cells/kg, 3×10⁶ cells/kg, 1×10⁷ cells/kg.

Participants are evaluated for toxicities related to the CAB-001 therapy and its effect on their underlying disease during an initial 28-day assessment period. Participants are monitored post-infusion, and subsequent visits occur approximately on Days 5, 8, 15, 22, and 29 post-infusion. In addition to routine chemistry and hematology tests, lab tests for PK sampling, coagulation panel, CRS labs, serum cytokine assessment, complement research assessment, and CAR T assessment are performed through Day 29. After the Day 29 post-infusion evaluation, participants undergo evaluations every 4 weeks (±7 days) in the first year, approximately every 12 weeks (+14 days) in the second year, and approximately once every 26 weeks (±30 days) in the third year. All participants are evaluated for safety, tolerability, efficacy, and quality of life for 156 weeks following infusion. Participants may also monitored for 15 years after treatment, as appropriate

### Inclusion and Exclusion Criteria

Inclusion criteria for participation in this study include: (1) able to provide informed consent; (2) 18 years of age or older, and 65 years of age or younger; (3) Eastern Cooperative Oncology Group (ECOG) performance status 0 to 1; (4) (EULAR)/American College of Rheumatology (ACR) classification criteria for adult SLE; (5) positive antinuclear antibody (ANA) titer ≥1:80 or positive anti-dsDNA antibody at screening; (6) for LN subjects only: active, biopsy-proven lupus nephritis class III or IV, with or without the presence of Class V, using the 2018 Revised International Society of Nephrology/Renal Pathology Society criteria (Bajema et al. (2018) KIDNEY INT. 93(4): 789-796), wherein the biopsy must be performed within 26 weeks prior to the screening visit or during the screening period; (7) diagnosed with active SLE, wherein (7a) For LN subjects: urine protein-to-creatinine ratio (UPCR) ≥1 mg/mg on 2 first morning void urine samples during screening despite prior or current treatment with standard of care therapy for at least 12 weeks, including corticosteroids, MMF/mycophenolic acid (MPA), CY, azathioprine, antimalarial therapy, belimumab, tacrolimus, cyclosporine, voclosporin, and/or rituximab; (7b) For non-renal SLE subjects: SLEDAI-2K≥8 and clinical SLEDAI-2K≥6 (excluding headache, alopecia, mucosal ulcers, fever, and organic brain syndrome) during screening despite prior or current treatment with standard of care therapy for at least 12 weeks, including corticosteroids, rituximab or other B cell depleting agents, CY, MMF/MPA, azathioprine, methotrexate, 6-mercaptopurine, sirolimus, tacrolimus, thalidomide, leflunomide, mizoribine, anifrolumab, and/or belimumab; (8a) if a subject is currently receiving standard immunosuppressive therapy (including MMF/MPA, CY, azathioprine, antimalarial therapy, methotrexate, 6-mercaptopurine, sirolimus, belimumab, anifrolumab, tacrolimus, cyclosporine, voclosporin, thalidomide, leflunomide, or mizoribine), the therapy must have been initiated at least 12 weeks prior to screening and on a stable dose for at least 8 weeks prior to screening, except for dose reduction due to safety or tolerability; (8b) if a subject is currently receiving a renin-angiotensin-aldosterone inhibitor, (including direct renin inhibitors, angiotensin converting enzyme (ACE) inhibitors, angiotensin receptor blockers (ARBs), and mineralocorticoid receptor blockers), the subject must be on a stable dose for at least 2 weeks prior to screening; (8c) if a subject is currently receiving a sodium-glucose cotransporter-2 (SGLT2) inhibitor, the subject must be on a stable dose for at least 2 weeks prior to screening; (8d) if a subject is currently receiving an oral corticosteroid, the subject must be receiving ≤30 mg prednisone or equivalent and on a stable dose for at least 2 weeks prior to screening; (9) adequate renal function, defined for LN subjects as estimated glomerular filtration rate of ≥45 mL/min/1.73 m² using the chronic kidney disease-epidemiology equation, and defined for non-renal SLE subjects as estimated glomerular filtration rate of ≥60 mL/min/1.73 m² using the chronic kidney disease-epidemiology equation; (10) adequate hepatic function as evidenced by alanine aminotransferase (ALT)≤1.5 times of the upper limits of normal; (12) have received all recommended vaccinations, including against COVID-19/severe acute respiratory syndrome coronavirus 2 (SARS-CoV-2), per the Centers for Disease Control and Prevention (CDC) or local/institutional guidelines for immunocompromised individuals before or during screening (live vaccines must be administered at least 30 days prior to pre-infusion visit, and non-live vaccines should be administered to subjects at least 2 weeks prior to the start of study drug infusion and, if possible, non-live vaccines should also be administered at least 2 weeks prior to leukapheresis); (12) clinical stability by vital signs assessment at the time of screening, including a. systolic blood pressure ≥90 mmHg and ≤170 mmHg, b. diastolic blood pressure ≥55 mmHg and ≤10⁵ mmHg, c. pulse ≥50 and ≤110 beats per minute, d. respiratory rate ≥12 and ≤20 breaths per minute, and e. afebrile; (13) women of reproductive potential (Section 9.4) must agree to use two acceptable methods of contraception from screening to a minimum of 52 weeks after the CAB-001 infusion, and men of reproductive potential are required to use condoms with spermicide from screening until a minimum of 52 weeks after CAB-001 infusion and must also agree not to donate sperm from screening to a minimum of 52 weeks after the CAB-001 infusion.

Exclusion criteria include patients you have any of the following criteria: (1) treatment with rituximab or other B cell-depleting agent within 26 weeks prior to screening, or within 12 weeks if there are laboratory results indicating presence of CD19+ B cells; 2. for LN subjects only: evidence of severe chronicity on kidney biopsy, defined as a modified National Institute of Health chronicity index score of 3+ for any of the following individual biopsy features: total glomerulosclerosis score, fibrous crescents, tubular atrophy, or interstitial fibrosis; (3) diagnosis of cancer, except basal or squamous cell skin cancer or carcinoma in situ of the cervix that has been excised and cured and at least 5 years since excision; (4) planned major surgery (including joint surgery) within 52 weeks following the CAB-001 infusion; (5) contraindication to leukapheresis; (6) history of anaphylactic or severe systemic reaction to fludarabine, cyclophosphamide, or any of their metabolites; (7) any of the following clinical laboratory values at screening: a. absolute lymphocyte count <500/μL, b. absolute neutrophil count <1,000/μL, c. hemoglobin <8 g/dL at screening or hemoglobin <7 g/dL if related to the subject's SLE such as active hemolytic anemia, or d. platelet count <80,000/μL; (8) A diagnosis of antiphospholipid antibody syndrome by the 2006 Revised Sapporo International Consensus Criteria at the time of screening (Miyakis et al. (2006) J. THROMB. HAEMOST. 4(2): 295-306); (9) positive human immunodeficiency virus (HIV), hepatitis C antibody, or hepatitis B surface antigen test, or evidence of active or chronic tuberculosis at screening (if hepatitis B surface antigen is negative with a positive hepatitis B core antibody, the subject is eligible but will require prophylaxis with high potency nucleoside or nucleotide analogues, starting prior to the preconditioning regimen and continuing at least 24 weeks after B cell repopulation; if the hepatitis C virus (HCV) antibody is positive, a confirmatory HCV ribonucleic acid (RNA) assay should be performed, and eligibility will be determined based on the result; an HCV antibody test is not required if documentation of a negative result of an HCV RNA test performed within 8 weeks prior to screening is provided; if the HIV screening test is positive, confirmatory testing (e.g., immunoassay and/or nucleic acid testing) should be performed per local guidelines); (10) active infection requiring medical intervention at screening; (11) autoimmune disorder other than SLE requiring immunosuppressive therapies; (12) the presence of kidney disease other than active lupus nephritis; (13) current symptoms of severe, progressive, or uncontrolled renal, hepatic, hematological, gastrointestinal, pulmonary, psychiatric, cardiac, neurological, or cerebral disease, including severe and uncontrolled infections, such as sepsis and opportunistic infections; (14) concomitant medical conditions that, in the opinion of the investigator, might place the subject at unacceptable risk for participation in this study, interfere with the assessment of the effects or safety of the investigational product or with the study procedures; (15) the presence of severe edema or anasarca at screening, defined as any of the following: a. the presence of localized (e.g., facial, limb, or pulmonary) edema, or generalized edema (e.g., anasarca) that interferes with the subject's self-care and/or activities of daily living, orb. the presence of skin breakdown due to edema; (16) moderate-to-severe chronic pulmonary disease, including: a. asthma requiring or refractory to medium or high-dose inhaled corticosteroids combined with other longer-acting medications, b. chronic obstructive pulmonary disease based on medical history, physical examination, imaging tests, and pulmonary function tests, c. requirement of supplemental oxygen d. resting oxygen saturation <92%, e. in subjects who have had pulmonary function tests: spirometry results of forced expiratory volume (FEV1)/forced vital capacity <0.7 and FEV1<80% predicted after bronchodilators, or diffusing capacity of the lungs for carbon monoxide results <60% predicted; (17) impaired cardiac function or clinically significant cardiac disease, including any of a. to f.: a. unstable angina or myocardial infarction or coronary artery bypass graft within 26 weeks prior to leukapheresis, b. New York Heart Association stage III or IV congestive heart failure. c. history of clinically significant cardiac arrhythmia (e.g., ventricular tachycardia), complete left bundle branch block, high-grade atrioventricular block, d. history of severe non-ischemic cardiomyopathy, e. left ventricular ejection fraction <45% as assessed by echocardiogram or multi-gated acquisition scan (if performed)≤8 weeks of leukapheresis, f. active, severe cardiac manifestations of SLE, including constrictive pericarditis, hemodynamically significant pericardial effusions, and myocarditis at the time of screening; (18) previous CAR T cell therapy; (19) prior solid organ (heart, liver, kidney, lung) transplant or hematopoietic cell transplant; (20) pregnant or lactating woman, or plan to become pregnant within 52 weeks following CAB-001 infusion; (21) men of reproductive potential (see Section 9.4) who plan to father a child in the 52 weeks following CAB-001 infusion; (22) unable or unwilling to comply with protocol; (23) treatment with any investigational agent within 4 weeks or 5 half-lives, whichever is longer (subjects who had received a C5 inhibitor (e.g., eculizumab, ravulizumab) may be eligible earlier than 5 half-lives after the last C5 inhibitor administration if, at screening, they have evidence of complement lab testing (i.e., total hemolytic complement [CH50] measurement) that has normalized or returned to pre-treatment levels.

It is contemplated that the CD19-CAR T cell therapy will be effective to treat SLE, including LN and non-renal SLE. It is further contemplated that the use of the reduced preconditioning regimen prior to treatment with CD19-CAR T cell therapy will be effective in the treatment of SLE, and that a standard, full-dose preconditioning regimen is not required.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

SEQUENCE LISTING

| SEQ | |
|---|---|
| ID NO: | Sequence |
| 1 | GYSFTSYW |
| 2 | YPGDSDT |
| 3 | ARLSYSWSSWYWDF |
| 4 | EVOLVQSGAEVKKPGESLKISCKGSGYSFTSYWIGWVRQMPGKGLEW |
| | MGIIYPGDSDTRYSPSFQGQVTISADKSISTAYLQWSSLKASDTAMYYC |
| | ARLSYSWSSWYWDFWGQGTLVTVSS |
| 5 | SSNIGNNA |
| 6 | YDD |
| 7 | AAWDDSLNGWV |
| 8 | QAVLTQPPSVSEAPRQRVTISCSGSSSNIGNNAVSWYQQLPGKAPKLLI |
| | YYDDLLPSGVSDRFSGSKSGTSASLAISGLQSEDEADYYCAAWDDSLN |
| | GWVFGGGTKVTVLG |
| 9 | QAVLTQPPSVSEAPRQRVTISCSGSSSNIGNNAVSWYQQLPGKAPKLLI |
| | YYDDLLPSGVSDRFSGSKSGTSASLAISGLQSEDEADYYCAAWDDSLN |
| | GWVFGGGTKVTVLGGGGGSGGGGSGGGGSEVQLVQSGAEVKKPGES |
| | LKISCKGSGYSFTSYWIGWVRQMPGKGLEWMGIIYPGDSDTRYSPSFQ |
| | GQVTISADKSISTAYLQWSSLKASDTAMYYCARLSYSWSSWYWDFWG |
| | QGTLVTVSS |
| 10 | GVSLPDYG |
| 11 | IWGSETT |
| 12 | AKHYYYGGSYAMDY |
| 13 | EVKLQESGPGLVAPSQSLSVTCTVSGVSLPDYGVSWIRQPPRKGLEWL |
| | GVIWGSETTYYNSALKSRLTIIKDNSKSQVFLKMNSLQTDDTAIYYCAK |
| | HYYYGGSYAMDYWGQGTSVTVSS |
| 14 | QDISKY |
| 15 | HTS |
| 16 | QQGNTLPYT |
| 17 | DIQMTQTTSSLSASLGDRVTISCRASQDISKYLNWYQQKPDGTVKLLIY |
| | HTSRLHSGVPSRFSGSGSGTDYSLTISNLEQEDIATYFCQQGNTLPYTFG |
| | GGTKLEIT |
| 18 | DIQMTQTTSSLSASLGDRVTISCRASQDISKYLNWYQQKPDGTVKLLIY |
| | HTSRLHSGVPSRFSGSGSGTDYSLTISNLEQEDIATYFCQQGNTLPYTFG |
| | GGTKLEITGSTSGSGKPGSGEGSTKGEVKLQESGPGLVAPSQSLSVTCT |
| | VSGVSLPDYGVSWIRQPPRKGLEWLGVIWGSETTYYNSALKSRLTIIKD |
| | NSKSQVFLKMNSLQTDDTAIYYCAKHYYYGGSYAMDYWGQGTSVTV |
| | SS |
| 19 | IYIWAPLAGTCGVLLLSLVITLYC |
| 20 | KRGRKKLLYIFKQPFMRPVQTTQEEDGCSCRFPEEEEGGCEL |
| 21 | RVKFSRSADAPAYQQGQNQLYNELNLGRREEYDVLDKRRGRDPEMGG |
| | KPRRKNPQEGLYNELQKDKMAEAYSEIGMKGERRRGKGHDGLYQGLS |
| | TATKDTYDALHMQALPPR |
| 22 | TTTPAPRPPTPAPTIASQPLSLRPEACRPAAGGAVHTRGLDFACD |
| 23 | QAVLTQPPSVSEAPRQRVTISCSGSSSNIGNNAVSWYQQLPGKAPKLLI |
| | YYDDLLPSGVSDRFSGSKSGTSASLAISGLQSEDEADYYCAAWDDSLN |
| | GWVFGGGTKVTVLGGGGGSGGGGSGGGGSEVQLVQSGAEVKKPGES |
| | LKISCKGSGYSFTSYWIGWVRQMPGKGLEWMGIIYPGDSDTRYSPSFQ |
| | GQVTISADKSISTAYLQWSSLKASDTAMYYCARLSYSWSSWYWDFWG |
| | QGTLVTVSSTTTPAPRPPTPAPTIASQPLSLRPEACRPAAGGAVHTRGLD |
| | FACDIYIWAPLAGTCGVLLLSLVITLYCKRGRKKLLYIFKQPFMRPVQT |
| | TQEEDGCSCRFPEEEEGGCELRVKFSRSADAPAYQQGQNQLYNELNLG |
| | RREEYDVLDKRRGRDPEMGGKPRRKNPQEGLYNELQKDKMAEAYSEI |
| | GMKGERRRGKGHDGLYQGLSTATKDTYDALHMQALPPR |
| 25 | MALPVTALLLPLALLLHAARP |
| 26 | MEFGLSWLFLVAILKGVQC |
| 27 | MALPVTALLLPLALLLHAARPQAVLTQPPSVSEAPRQRVTISCSGSSSNI |
| | GNNAVSWYQQLPGKAPKLLIYYDDLLPSGVSDRFSGSKSGTSASLAISG |
| | LQSEDEADYYCAAWDDSLNGWVFGGGTKVTVLGGGGGSGGGGSGGG |
| | GSEVQLVQSGAEVKKPGESLKISCKGSGYSFTSYWIGWVRQMPGKGLE |
| | WMGIIYPGDSDTRYSPSFQGQVTISADKSISTAYLQWSSLKASDTAMYY |
| | CARLSYSWSSWYWDFWGQGTLVTVSSTTTPAPRPPTPAPTIASQPLSLR |
| | PEACRPAAGGAVHTRGLDFACDIYIWAPLAGTCGVLLLSLVITLYCKRG |
| | RKKLLYIFKQPFMRPVQTTQEEDGCSCRFPEEEEGGCELRVKFSRSADA |
| | PAYQQGQNQLYNELNLGRREEYDVLDKRRGRDPEMGGKPRRKNPQE |
| | GLYNELQKDKMAEAYSEIGMKGERRRGKGHDGLYQGLSTATKDTYD |
| | ALHMQALPPR |
| 28 | CAGGCTGTGCTGACTCAGCCACCCTCGGTGTCTGAAGCCCCCAGGC |
| | AGAGGGTCACCATCTCCTGTTCTGGAAGCAGCTCCAACATCGGAAA |
| | TAATGCTGTAAGCTGGTACCAGCAGCTCCCAGGAAAGGCTCCCAAA |
| | CTCCTCATCTATTATGATGATCTGCTCCCCTCAGGGGTCTCTGACCG |
| | ATTCTCTGGCTCCAAGTCTGGCACCTCAGCCTCCCTGGCCATCAGTG |
| | GGCTCCAGTCTGAGGATGAGGCTGATTATTACTGTGCAGCATGGGA |
| | TGACAGCCTGAATGGTTGGGTGTTCGGCGGAGGGACCAAGGTCACC |
| | GTCCTAGGTGGTGGTGGTGGTAGCGGCGGCGGCGGCTCTGGTGGTG |
| | GTGGGTCCGAGGTGCAGCTGGTGCAGTCTGGAGCAGAGGTGAAAAA |
| | GCCCGGGGAGTCTCTGAAGATCTCCTGTAAGGGTTCTGGATACAGCT |
| | TTACCAGCTACTGGATCGGCTGGGTGCGCCAGATGCCCGGGAAAGG |
| | CCTGGAGTGGATGGGGATCATCTATCCTGGTGACTCTGATACCAGAT |
| | ACAGCCCGTCCTTCCAAGGCCAGGTCACCATCTCAGCCGACAAGTC |
| | CATCAGCACCGCCTACCTGCAGTGGAGCAGCCTGAAGGCCTCGGAC |
| | ACCGCCATGTATTACTGTGCGCGCCTGTCTTACTCTTGGTCTTCTTGG |
| | TACTGGGATTTCTGGGGTCAAGGTACTCTGGTGACCGTCTCCTCAAC |
| | CACCACTCCCGCACCAAGACCACCAACTCCAGCCCCCACTATCGCAT |
| | CTCAGCCACTCTCACTTCGGCCTGAAGCTTGTAGACCTGCGGCGGGC |
| | GGTGCAGTTCATACGAGGGGCTTGGATTTTGCCTGCGATATATACAT |
| | ATGGGCACCGTTGGCAGGTACGTGCGGGGTGCTCCTCCTGAGTCTG |
| | GTTATAACACTCTATTGCAAACGGGGCAGAAAGAAACTCCTGTATA |
| | TATTCAAACAACCATTTATGAGACCAGTACAAACTACTCAAGAGGA |
| | AGATGGCTGTAGCTGCCGATTTCCAGAAGAAGAAGAAGGAGGATGT |
| | GAACTGAGAGTGAAGTTCAGCAGGAGCGCAGACGCCCCCGCGTACC |
| | AGCAGGGCCAGAACCAGCTCTATAACGAGCTCAATCTAGGACGAAG |
| | AGAGGAGTACGATGTTTTGGACAAGAGACGTGGCCGGGACCCTGAG |
| | ATGGGGGGAAAGCCGAGAAGGAAGAACCCTCAGGAAGGCCTGTAC |
| | AATGAACTGCAGAAAGATAAGATGGCGGAGGCCTACAGTGAGATTG |
| | GGATGAAAGGCGAGCGCCGGAGGGGCAAGGGGCACGATGGCCTTT |
| | ACCAGGGTCTCAGTACAGCCACCAAGGACACCTACGACGCCCTTCA |
| | CATGCAGGCCCTGCCCCCTCGC |
| 29 | ATGGCCTTACCAGTGACCGCCTTGCTCCTGCCGCTGGCCTTGCTGCT |
| | CCACGCCGCCAGGCCGCAGGCTGTGCTGACTCAGCCACCCTCGGTG |
| | TCTGAAGCCCCCAGGCAGAGGGTCACCATCTCCTGTTCTGGAAGCA |
| | GCTCCAACATCGGAAATAATGCTGTAAGCTGGTACCAGCAGCTCCC |
| | AGGAAAGGCTCCCAAACTCCTCATCTATTATGATGATCTGCTCCCCT |
| | CAGGGGTCTCTGACCGATTCTCTGGCTCCAAGTCTGGCACCTCAGCC |
| | TCCCTGGCCATCAGTGGGCTCCAGTCTGAGGATGAGGCTGATTATTA |
| | CTGTGCAGCATGGGATGACAGCCTGAATGGTTGGGTGTTCGGCGGA |
| | GGGACCAAGGTCACCGTCCTAGGTGGTGGTGGTGGTAGCGGCGGCG |
| | GCGGCTCTGGTGGTGGTGGGTCCGAGGTGCAGCTGGTGCAGTCTGG |
| | AGCAGAGGTGAAAAAGCCCGGGGAGTCTCTGAAGATCTCCTGTAAG |
| | GGTTCTGGATACAGCTTTACCAGCTACTGGATCGGCTGGGTGCGCCA |
| | GATGCCCGGGAAAGGCCTGGAGTGGATGGGGATCATCTATCCTGGT |
| | GACTCTGATACCAGATACAGCCCGTCCTTCCAAGGCCAGGTCACCAT |
| | CTCAGCCGACAAGTCCATCAGCACCGCCTACCTGCAGTGGAGCAGC |
| | CTGAAGGCCTCGGACACCGCCATGTATTACTGTGCGCGCCTGTCTTA |
| | CTCTTGGTCTTCTTGGTACTGGGATTTCTGGGGTCAAGGTACTCTGG |
| | TGACCGTCTCCTCAACCACCACTCCCGCACCAAGACCACCAACTCCA |
| | GCCCCCACTATCGCATCTCAGCCACTCTCACTTCGGCCTGAAGCTTG |
| | TAGACCTGCGGCGGGCGGTGCAGTTCATACGAGGGGCTTGGATTTT |
| | GCCTGCGATATATACATATGGGCACCGTTGGCAGGTACGTGCGGGG |
| | TGCTCCTCCTGAGTCTGGTTATAACACTCTATTGCAAACGGGGCAGA |
| | AAGAAACTCCTGTATATATTCAAACAACCATTTATGAGACCAGTAC |
| | AAACTACTCAAGAGGAAGATGGCTGTAGCTGCCGATTTCCAGAAGA |
| | AGAAGAAGGAGGATGTGAACTGAGAGTGAAGTTCAGCAGGAGCGC |
| | AGACGCCCCCGCGTACCAGCAGGGCCAGAACCAGCTCTATAACGAG |
| | CTCAATCTAGGACGAAGAGAGGAGTACGATGTTTTGGACAAGAGAC |
| | GTGGCCGGGACCCTGAGATGGGGGGAAAGCCGAGAAGGAAGAACC |
| | CTCAGGAAGGCCTGTACAATGAACTGCAGAAAGATAAGATGGCGGA |
| | GGCCTACAGTGAGATTGGGATGAAAGGCGAGCGCCGGAGGGGCAA |
| | GGGGCACGATGGCCTTTACCAGGGTCTCAGTACAGCCACCAAGGAC |
| | ACCTACGACGCCCTTCACATGCAGGCCCTGCCCCCTCGC |
| 30 | GYSFTSY |
| 31 | YPGDSD |
| 32 | LSYSWSSWYWDF |
| 33 | SGSSSNIGNNAVS |
| 34 | YDDLLPS |
| 35 | [Intentionally blank] |
| 36 | GVSLPDY |
| 37 | WGSET |
| 38 | HYYYGGSYAMDY |
| 39 | RASQDISKYLN |
| 40 | HTSRLHS |
| 41 | GGTFSSYA |
| 42 | IIPIFGTT |
| 43 | AREAVAADWLDP |
| 44 | QVQLVQSGAEVKKPGSSVKVSCKDSGGTFSSYAISWVRQAPGQGLEW |
| | MGGIIPIFGTTNYAQQFQGRVTITADESTSTAYMELSSLRSEDTAVYYC |
| | AREAVAADWLDPWGQGTLVTVSS |
| 45 | QSVSSSY |
| 46 | GAS |
| 47 | QQYGSSRFT |
| 48 | EIVLTQSPGTLSLSPGERATLSCRASQSVSSSYLAWYQQKPGQAPRLLIY |
| | GASSRATGIPDRFSGSGSGTDFTLTISRLEPEDFAVYYCQQYGSSRFTFG |
| | PGTKVDIK |
| 49 | GGTFSSY |
| 50 | IPIFGT |
| 51 | EAVAADWLDP |
| 52 | RASQSVSSSYLA |
| 53 | GASSRAT |
| 54 | SYWIG |
| 55 | IIYPGDSDTRYSPSFQG |
| 56 | DYGVS |
| 57 | VIWGSETTYYNSALKS |
| 58 | SYAIS |
| 59 | GIIPIFGTTNYAQQFQG |
| 60 | EIVLTQSPGTLSLSPGERATLSCRASQSVSSSYLAWYQQKPGQAPRLLIY |
| | GASSRATGIPDRFSGSGSGTDFTLTISRLEPEDFAVYYCQQYGSSRFTFG |
| | PGTKVDIKGSTSGSGKPGSGEGSTKGQVQLVQSGAEVKKPGSSVKVSC |
| | KDSGGTFSSYAISWVRQAPGQGLEWMGGIIPIFGTTNYAQQFQGRVTIT |
| | ADESTSTAYMELSSLRSEDTAVYYCAREAVAADWLDPWGQGTLVTVS |
| | S |
| 61 | FWVLVVVGGVLACYSLLVTVAFIIFWV |
| 62 | RSKRSRLLHSDYMNMTPRRPGPTRKHYQPYAPPRDFAAYRS |
| 63 | EIVLTQSPGTLSLSPGERATLSCRASQSVSSSYLAWYQQKPGQAPRLLIY |
| | GASSRATGIPDRFSGSGSGTDFTLTISRLEPEDFAVYYCQQYGSSRFTFG |
| | PGTKVDIKGSTSGSGKPGSGEGSTKGQVQLVQSGAEVKKPGSSVKVSC |
| | KDSGGTFSSYAISWVRQAPGQGLEWMGGIIPIFGTTNYAQQFQGRVTIT |
| | ADESTSTAYMELSSLRSEDTAVYYCAREAVAADWLDPWGQGTLVTVS |
| | SFVPVFLPAKPTTTPAPRPPTPAPTIASQPLSLRPEACRPAAGGAVHTRG |
| | LDFACDIYIWAPLAGTCGVLLLSLVITLYCNHRNRSKRSRLLHSDYMN |
| | MTPRRPGPTRKHYQPYAPPRDFAAYRSRVKFSRSADAPAYQQGQNQL |
| | YNELNLGRREEYDVLDKRRGRDPEMGGKPRRKNPQEGLYNELQKDK |
| | MAEAYSEIGMKGERRRGKGHDGLYQGLSTATKDTYDALHMQALPPR |
| 64 | MALPVTALLLPLALLLHAARPEIVLTQSPGTLSLSPGERATLSCRASQSV |
| | SSSYLAWYQQKPGQAPRLLIYGASSRATGIPDRFSGSGSGTDFTLTISRL |
| | EPEDFAVYYCQQYGSSRFTFGPGTKVDIKGSTSGSGKPGSGEGSTKGQV |
| | QLVQSGAEVKKPGSSVKVSCKDSGGTFSSYAISWVRQAPGQGLEWMG |
| | GIIPIFGTTNYAQQFQGRVTITADESTSTAYMELSSLRSEDTAVYYCARE |
| | AVAADWLDPWGQGTLVTVSSFVPVFLPAKPTTTPAPRPPTPAPTIASQP |
| | LSLRPEACRPAAGGAVHTRGLDFACDIYIWAPLAGTCGVLLLSLVITLY |
| | CNHRNRSKRSRLLHSDYMNMTPRRPGPTRKHYQPYAPPRDFAAYRSR |
| | VKFSRSADAPAYQQGQNQLYNELNLGRREEYDVLDKRRGRDPEMGG |
| | KPRRKNPQEGLYNELQKDKMAEAYSEIGMKGERRRGKGHDGLYQGLS |
| | TATKDTYDALHMQALPPR |

What is claimed is:

1. A method of treating an autoimmune disease in a subject in need thereof, the method comprising:
(a) administering to the subject a preconditioning regimen comprising a dose of 100-1,500 mg/m²/day cyclophosphamide and a dose of 2-20 mg/m²/day fludarabine; and
(b) after step (a), administering to the subject a therapeutically effective amount of engineered CAR immune cells.

2. The method of claim 1, wherein the dose of cyclophosphamide is less than 1,000 mg/m²/day.

3. The method of claim 1 or claim 2, wherein the dose of cyclophosphamide is less than 800 mg/m²/day.

4. The method of any one of claims 1-3, wherein the dose of cyclophosphamide is less than 600 mg/m²/day.

5. The method of any one of claims 1-4, wherein the dose of cyclophosphamide is less than 500 mg/m²/day.

6. The method of any one of claims 1-5, wherein the dose of cyclophosphamide is 200-800 mg/m²/day.

7. The method of any one of claims 1-6, wherein the dose of cyclophosphamide is 400-600 mg/m²/day.

8. The method of claim 1, wherein the dose of cyclophosphamide is 1,000 mg/m²/day.

9. The method of any one of claims 1-7, wherein the dose of cyclophosphamide is 500 mg/m²/day.

10. The method of any one of claims 1-6, wherein the dose of cyclophosphamide is 250 mg/m²/day.

11. The method of any one of claims 1-10, wherein the preconditioning regimen comprises administering a total dose of cyclophosphamide of less than 2,000 mg/m², less than 1,000 mg/m², less than 800 mg/m², less than 600 mg/m², or less than 500 mg/m² to the subject.

12. The method of any one of claims 1-10, wherein the preconditioning regimen comprises administering a total dose of 100-1,500 mg/m² cyclophosphamide to the subject.

13. The method of any one of claims 1-10, wherein the preconditioning regimen comprises administering a total dose of 250-1,000 mg/m² cyclophosphamide to the subject.

14. The method of any one of claims 1-7 or 9-10, wherein the preconditioning regimen comprises administering a total dose of 500 mg/m² cyclophosphamide to the subject.

15. The method of any one of claims 1-6 or 10, wherein the preconditioning regimen comprises administering a total dose of 250 mg/m² cyclophosphamide to the subject.

16. The method of any one of claims 1-10, wherein the preconditioning regimen comprises administering a total dose of 1,000 mg/m² cyclophosphamide to the subject.

17. The method of any one of claims 1-16, wherein the subject is administered less than 2,000 mg/m², less than 1,000 mg/m², less than 800 mg/m², less than 600 mg/m², or less than 500 mg/m² cyclophosphamide in total during the seven days prior to step (b).

18. The method of any one of claims 1-17, wherein the dose of fludarabine is less than 15 mg/m²/day.

19. The method of any one of claims 1-18, wherein the dose of fludarabine is less than 12.5 mg/m²/day.

20. The method of any one of claims 1-19, wherein the dose of fludarabine is less than 10 mg/m²/day.

21. The method of any one of claims 1-20, wherein the dose of fludarabine is 5-15 mg/m²/day.

22. The method of any one of claims 1-21, wherein the dose of fludarabine is 10-15 mg/m²/day.

23. The method of any one of claims 1-17, wherein the dose of fludarabine is 15 mg/m²/day.

24. The method of any one of claims 1-18, wherein the dose of fludarabine is 12.5 mg/m²/day.

25. The method of any one of claims 1-21, wherein the dose of fludarabine is 6.25 mg/m²/day.

26. The method of any one of claims 1-25, wherein the preconditioning regimen comprises administering a total dose of fludarabine of less than 75 mg/m², less than 50 mg/m², or less than 25 mg/m² to the subject.

27. The method of any one of claims 1-25, wherein the preconditioning regimen comprises administering a total dose of 5-60 mg/m² fludarabine to the subject.

28. The method of any one of claims 1-25, wherein the preconditioning regimen comprises administering a total dose of 15-50 mg/m² fludarabine to the subject.

29. The method of any one of claims 1-25, wherein the preconditioning regimen comprises administering a total dose of 37.5 mg/m² fludarabine to the subject.

30. The method of any one of claims 1-25, wherein the preconditioning regimen comprises administering a total dose of 18.75 mg/m² fludarabine to the subject.

31. The method of any one of claims 1-25, wherein the preconditioning regimen comprises administering a total dose of 45 mg/m² fludarabine to the subject.

32. The method of any one of claims 1-31, wherein the subject is administered less than less than 75 mg/m², less than 50 mg/m², or less than 25 mg/m² fludarabine in total during the seven days prior to step (b).

33. The method of any one of claims 1-32, wherein the dose of cyclophosphamide and/or the dose of fludarabine are administered between 1 and 7 days prior to step (b).

34. The method of any one of claims 1-33, wherein the dose of cyclophosphamide and the dose of fludarabine are administered on the same day.

35. The method of any one of claims 1-34, wherein the dose of cyclophosphamide is administered to the subject 3 days and/or 4 days prior to step (b).

36. The method of any one of claims 1-35, wherein the dose of fludarabine is administered daily for three days prior to step (b).

37. The method of claim 36, wherein the dose of fludarabine is administered 5 days, 4 days, and/or 3 days prior to step (b).

38. The method of claim 1, wherein the preconditioning regimen comprises (i) administering the dose of fludarabine to the subject 5 days, 4 days, and 3 days prior to step (b), and (ii) administering the dose of cyclophosphamide to the subject 3 days prior to step (b).

39. The method of claim 1, wherein the preconditioning regimen consists essentially of (i) administering the dose of fludarabine to the subject 5 days, 4 days, and 3 days prior to step (b), and (ii) administering the dose of cyclophosphamide to the subject 3 days prior to step (b).

40. The method of claim 38 or claim 39, wherein the dose of fludarabine is 12.5 mg/m²/day and the dose of cyclophosphamide is 500 mg/m²/day.

41. The method of claim 38 or claim 39, wherein the dose of fludarabine is 15 mg/m²/day and the dose of cyclophosphamide is 500 mg/m²/day.

42. The method of claim 38 or claim 39, wherein the dose of fludarabine is 12.5 mg/m²/day and the dose of cyclophosphamide is 1,000 mg/m²/day.

43. The method of claim 38 or claim 39, wherein the dose of fludarabine is 6.25 mg/m²/day and the dose of cyclophosphamide is 250 mg/m²/day.

44. The method of claim 38 or claim 39, wherein the dose of fludarabine is 6.25 mg/m²/day and the dose of cyclophosphamide is 500 mg/m²/day.

45. The method of claim 38 or claim 39, wherein the dose of fludarabine is 6.25 mg/m²/day and the dose of cyclophosphamide is 1,000 mg/m²/day.

46. The method of claim 1, wherein the preconditioning regimen comprises (i) administering the dose of fludarabine to the subject 5 days, 4 days, and 3 days prior to step (b), and (ii) administering the dose of cyclophosphamide to the subject 4 days and 3 days prior to step (b).

47. The method of claim 1, wherein the preconditioning regimen consists essentially of (i) administering the dose of fludarabine to the subject 5 days, 4 days, and 3 days prior to step (b), and (ii) administering the dose of cyclophosphamide to the subject 4 days and 3 days prior to step (b).

48. The method of claim 46 or 47, wherein the dose of fludarabine is 6.25 mg/m²/day and the dose of cyclophosphamide is 500 mg/m²/day.

49. The method of claim 1, wherein the preconditioning regimen is a regimen as described in Table 1.

50. The method of any one of claims 1-49, wherein the therapeutically effective amount of engineered CAR immune cells is between 1×10⁵ cells/kg and 1×10⁸ cells/kg.

51. The method of any one of claims 1-50, wherein the therapeutically effective amount of engineered CAR immune cells is between 1×10⁶ cells/kg and 1×10⁷ cells/kg.

52. The method of any one of claims 1-51, wherein the engineered CAR immune cells express a CAR which specifically binds a B-cell surface antigen.

53. The method of claim 52, wherein the B-cell surface antigen is CD19.

54. The method of any one of claims 1-53, wherein the engineered CAR immune cells comprise a nucleic acid encoding a CAR, wherein the CAR comprises:
(i) an extracellular domain comprising an antigen binding site;
(ii) a transmembrane domain;
(iii) a costimulatory domain; and
(iv) an intracellular signaling domain.

55. The method of claim 54, wherein the antigen binding site is defined by an scFv.

56. The method of claim 54 or claim 55, wherein the antigen binding site comprises a heavy chain variable domain (VH) comprising complementarity determining regions CDR_{H1}, CDR_{H2}, and CDR_{H3} and a light chain variable domain (VL) comprising complementarity determining regions CDR_{L1}, CDR_{L2}, and CDR_{L3}, wherein:
(i) the CDR_{H1}, CDR_{H2}, and CDR_{H3} comprise the amino acid sequences of SEQ ID NOs: 1, 2, and 3, respectively; and/or the CDR_{L1}, CDR_{L2}, and CDR_{L3} comprise the amino acid sequences of SEQ ID NOs: 5, 6, and 7, respectively; or
(ii) the CDR_{H1}, CDR_{H2}, and CDR_{H3} comprise the amino acid sequences of SEQ ID NOs: 10, 11, and 12, respectively; and/or the CDR_{L1}, CDR_{L2}, and CDR_{L3} comprise the amino acid sequences of SEQ ID NOs: 14, 15, and 16, respectively.

57. The method of claim 56, wherein:
(i) the VH and the VL comprise amino acid sequences at least 95% identical to the amino acid sequences of SEQ ID NOs: 4 and 8, respectively; or
(ii) the VH and the VL comprise amino acid sequences at least 95% identical to the amino acid sequences of SEQ ID NOs: 13 and 17, respectively.

58. The method of claim 56 or 57, wherein:
(i) the VH and the VL comprise the amino acid sequences SEQ ID NOs: 4 and 8, respectively; or
(ii) the VH and the VL comprise the amino acid sequences of SEQ ID NOs: 13 and 17, respectively.

59. The method of any one of claims 55-58, wherein the scFv comprises an amino acid sequence at least 95% identical to the amino acid sequence of SEQ ID NO: 9 or SEQ ID NO: 18.

60. The method of any one of claims 55-59, wherein the scFv comprises the amino acid sequence of SEQ ID NO: 9 or SEQ ID NO: 18.

61. The method of any one of claims 54-60, wherein the antigen binding site is humanized or fully human.

62. The method of any one of claims 54-61, wherein the transmembrane domain comprises a CD8 alpha chain transmembrane domain.

63. The method of claim 62, wherein the CD8 alpha chain transmembrane domain comprises the amino acid sequence of SEQ ID NO: 19.

64. The method of any one of claims 54-63, wherein the costimulatory domain comprises a 4-1BB intracellular domain.

65. The method of claim 64, wherein the 4-1BB intracellular domain comprises the amino acid sequence of SEQ ID NO: 20.

66. The method of any one of claims 54-65, wherein the intracellular signaling domain comprises a CD3ζ signaling domain.

67. The method of claim 66, wherein the CD3ζ signaling domain comprises the amino acid sequence of SEQ ID NO: 21.

68. The method of any one of claims 54-67, wherein the CAR further comprises a hinge or linker interposed between the antigen binding site and transmembrane domain.

69. The method of claim 68, wherein the hinge domain is a CD8 alpha chain hinge.

70. The method of claim 69, wherein the CD8 alpha chain hinge comprises the amino acid sequence of SEQ ID NO: 22.

71. The method of any one of claims 54-70, wherein the CAR comprises the amino acid sequence of SEQ ID NO: 23.

72. The method of any one of claims 1-71, wherein the autoimmune disease is a B-cell-mediated autoimmune disease.

73. The method of claim 72, wherein the autoimmune disease is selected from the group consisting of systemic lupus erythematosus (SLE), pemphigus vulgaris (PV), myasthenia gravis (MG), myositis, and membranous nephropathy.

74. The method of claim 72 or claim 73, wherein the autoimmune disease is selected from the group consisting of lupus nephritis, SLE with anti-dsDNA antibodies, mucosal PV, mucocutaneous PV, MuSK-associated MG, AChR MG, anti-synthetase syndrome, dermatomyositis, and immune mediated necrotizing myopathy.

75. The method of any one of claims 1-74, wherein the autoimmune disease is SLE.

76. The method of any one of claims 1-75, wherein the CAR immune cells are CAR T cells or CAR NK cells.

77. The method of any one of claims 1-76, wherein the CAR immune cells are CAR T cells.


SEQUENCE LISTING (64 sequences)

SEQ ID NO: 1 - protein, 8 aa, synthetic construct
GYSFTSYW

SEQ ID NO: 2 - protein, 7 aa, synthetic construct
YPGDSDT

SEQ ID NO: 3 - protein, 14 aa, synthetic construct
ARLSYSWSSWYWDF

SEQ ID NO: 4 - protein, 121 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSYSWSSWYWDFWGQGTLVTVSS

SEQ ID NO: 5 - protein, 8 aa, synthetic construct
SSNIGNNA

SEQ ID NO: 6 - skipped

SEQ ID NO: 7 - protein, 11 aa, synthetic construct
AAWDDSLNGWV

SEQ ID NO: 8 - protein, 111 aa, synthetic construct
QAVLTQPPSVSEAPRQRVTISCSGSSSNIGNNAVSWYQQLPGKAPKLLIYYDDLLPSGVSDRFSGSKSGTSASLAISGLQSEDEADYYCAAWDDSLNGWVFGGGTKVTVLG

SEQ ID NO: 9 - protein, 247 aa, synthetic construct
QAVLTQPPSVSEAPRQRVTISCSGSSSNIGNNAVSWYQQLPGKAPKLLIYYDDLLPSGVSDRFSGSKSGTSASLAISGLQSEDEADYYCAAWDDSLNGWVFGGGTKVTVLGGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSYSWSSWYWDFWGQGTLVTVSS

SEQ ID NO: 10 - protein, 8 aa, synthetic construct
GVSLPDYG

SEQ ID NO: 11 - protein, 7 aa, synthetic construct
IWGSETT

SEQ ID NO: 12 - protein, 14 aa, synthetic construct
AKHYYYGGSYAMDY

SEQ ID NO: 13 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 14 - protein, 6 aa, synthetic construct
QDISKY

SEQ ID NO: 15 - skipped

SEQ ID NO: 16 - protein, 9 aa, synthetic construct
QQGNTLPYT

SEQ ID NO: 17 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 18 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 19 - protein, 24 aa, Homo sapiens
IYIWAPLAGTCGVLLLSLVITLYC

SEQ ID NO: 20 - protein, 42 aa, Homo sapiens
KRGRKKLLYIFKQPFMRPVQTTQEEDGCSCRFPEEEEGGCEL

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 45 aa, Homo sapiens
TTTPAPRPPTPAPTIASQPLSLRPEACRPAAGGAVHTRGLDFACD

SEQ ID NO: 23 - protein, 470 aa, synthetic construct
QAVLTQPPSVSEAPRQRVTISCSGSSSNIGNNAVSWYQQLPGKAPKLLIYYDDLLPSGVSDRFSGSKSGTSASLAISGLQSEDEADYYCAAWDDSLNGWVFGGGTKVTVLGGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSYSWSSWYWDFWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - skipped

SEQ ID NO: 25 - protein, 21 aa, Homo sapiens
MALPVTALLLPLALLLHAARP

SEQ ID NO: 26 - protein, 19 aa, Homo sapiens
MEFGLSWLFLVAILKGVQC

SEQ ID NO: 27 - protein, 491 aa, synthetic construct
MALPVTALLLPLALLLHAARPQAVLTQPPSVSEAPRQRVTISCSGSSSNIGNNAVSWYQQLPGKAPKLLIYYDDLLPSGVSDRFSGSKSGTSASLAISGLQSEDEADYYCAAWDDSLNGWVFGGGTKVTVLGGGGGSGGGGSGGGGSEVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLSYSWSSWYWDFWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 1410 bp, synthetic construct
  source 1..1410: mol_type = other DNA
caggctgtgctgactcagccaccctcggtgtctgaagcccccaggcagagggtcaccatctcctgttctggaagcagctccaacatcggaaataatgctgtaagctggtaccagcagctcccaggaaaggctcccaaactcctcatctattatgatgatctgctcccctcaggggtctctgaccgattctctggctccaagtctggcacctcagcctccctggccatcagtgggctccagtctgaggatgaggctgattattactgtgcagcatgggatgacagcctgaatggttgggtgttcggcggagggaccaaggtcaccgtcctaggtggtggtggtggtagcggcggcggcggctctggtggtggtgggtccgaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgcgcctgtcttactcttggtcttcttggtactgggatttctggggtcaaggtactctggtgaccgtctcctcaaccaccactcccgcaccaagaccaccaactccagcccccactatcgcatctcagccactctcacttcggcctgaagcttgtagacctgcggcgggcggtgcagttcatacgaggggcttggattttgcctgcgatatatacatatgggcaccgttggcaggtacgtgcggggtgctcctcctgagtctggttataacactctattgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 29 - DNA, 1473 bp, synthetic construct
  source 1..1473: mol_type = other DNA
atggccttaccagtgaccgccttgctcctgccgctggccttgctgctccacgccgccaggccgcaggctgtgctgactcagccaccctcggtgtctgaagcccccaggcagagggtcaccatctcctgttctggaagcagctccaacatcggaaataatgctgtaagctggtaccagcagctcccaggaaaggctcccaaactcctcatctattatgatgatctgctcccctcaggggtctctgaccgattctctggctccaagtctggcacctcagcctccctggccatcagtgggctccagtctgaggatgaggctgattattactgtgcagcatgggatgacagcctgaatggttgggtgttcggcggagggaccaaggtcaccgtcctaggtggtggtggtggtagcggcggcggcggctctggtggtggtgggtccgaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgcgcctgtcttactcttggtcttcttggtactgggatttctggggtcaaggtactctggtgaccgtctcctcaaccaccactcccgcaccaagaccaccaactccagcccccactatcgcatctcagccactctcacttcggcctgaagcttgtagacctgcggcgggcggtgcagttcatacgaggggcttggattttgcctgcgatatatacatatgggcaccgttggcaggtacgtgcggggtgctcctcctgagtctggttataacactctattgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 30 - protein, 7 aa, synthetic construct
GYSFTSY

SEQ ID NO: 31 - protein, 6 aa, synthetic construct
YPGDSD

SEQ ID NO: 32 - protein, 12 aa, synthetic construct
LSYSWSSWYWDF

SEQ ID NO: 33 - protein, 13 aa, synthetic construct
SGSSSNIGNNAVS

SEQ ID NO: 34 - protein, 7 aa, synthetic construct
YDDLLPS

SEQ ID NO: 35 - skipped

SEQ ID NO: 36 - protein, 7 aa, synthetic construct
GVSLPDY

SEQ ID NO: 37 - protein, 5 aa, synthetic construct
WGSET

SEQ ID NO: 38 - protein, 12 aa, synthetic construct
HYYYGGSYAMDY

SEQ ID NO: 39 - protein, 11 aa, synthetic construct
RASQDISKYLN

SEQ ID NO: 40 - protein, 7 aa, synthetic construct
HTSRLHS

SEQ ID NO: 41 - protein, 8 aa, synthetic construct
GGTFSSYA

SEQ ID NO: 42 - protein, 8 aa, synthetic construct
IIPIFGTT

SEQ ID NO: 43 - protein, 12 aa, synthetic construct
AREAVAADWLDP

SEQ ID NO: 44 - protein, 119 aa, synthetic construct
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 45 - protein, 7 aa, synthetic construct
QSVSSSY

SEQ ID NO: 46 - skipped

SEQ ID NO: 47 - protein, 9 aa, synthetic construct
QQYGSSRFT

SEQ ID NO: 48 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 49 - protein, 7 aa, synthetic construct
GGTFSSY

SEQ ID NO: 50 - protein, 6 aa, synthetic construct
IPIFGT

SEQ ID NO: 51 - protein, 10 aa, synthetic construct
EAVAADWLDP

SEQ ID NO: 52 - protein, 12 aa, synthetic construct
RASQSVSSSYLA

SEQ ID NO: 53 - protein, 7 aa, synthetic construct
GASSRAT

SEQ ID NO: 54 - protein, 5 aa, synthetic construct
SYWIG

SEQ ID NO: 55 - protein, 17 aa, synthetic construct
IIYPGDSDTRYSPSFQG

SEQ ID NO: 56 - protein, 5 aa, synthetic construct
DYGVS

SEQ ID NO: 57 - protein, 16 aa, synthetic construct
VIWGSETTYYNSALKS

SEQ ID NO: 58 - protein, 5 aa, synthetic construct
SYAIS

SEQ ID NO: 59 - protein, 17 aa, synthetic construct
GIIPIFGTTNYAQQFQG

SEQ ID NO: 60 - protein, 245 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 61 - protein, 27 aa, Homo sapiens
FWVLVVVGGVLACYSLLVTVAFIIFWV

SEQ ID NO: 62 - protein, 41 aa, Homo sapiens
RSKRSRLLHSDYMNMTPRRPGPTRKHYQPYAPPRDFAAYRS

SEQ ID NO: 63 - protein, 481 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - protein, 502 aa, synthetic construct
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR